(19) United States
(12) Patent Application Publication
(10) Pub. No.: US 2007/0149763 A1
(43) Pub. Date: Jun. 28, 2007

(54) Beta secretase exosite binding peptides and methods for identifying beta secretase modulators

(75) Inventors: Michael G. Kornacker, Princeton, NJ (US); Robert A. Copeland, Hockessin, DE (US); Joseph P. Hendrick, Bridgeport, CT (US); Zhihong Lai, West Chester, PA (US); Claudio Mapelli, Plainsboro, NJ (US); Mark R. Witmer, Pennington, NJ (US); Jovita Marcinkeviciene, Washington Crossing, PA (US); William Joseph Metzler, Doylestown, PA (US); Ving G. Lee, Hamilton, NJ (US); Douglas James Riexinger, Flemington, NJ (US); Jody K. Muckelbauer, Moorestown, NJ (US); Chiehying Jean Chang, West Windsor, NJ (US); Daniel M. Camac, Wilmington, DE (US); Paul E. Morin, Pennington, NJ (US)

Correspondence Address: LOUIS J. WILLE;BRISTOL-MYERS SQUIBB COMPANY, PATENT DEPARTMENT, P O BOX 4000, PRINCETON, NJ, 08543-4000, US

(73) Assignee: Bristol-Myers Squibb Company

(21) Appl. No.: 11/649,035

(22) Filed: Jan. 3, 2007

Related U.S. Application Data
(63) Continuation-in-part of application No. 10/685,898, filed on Oct. 15, 2003.

(51) Int. Cl. C07K 7/08; C07K 7/06

(52) U.S. Cl. 530/326; 530/329; 530/330; 530/328; 530/327

(57) ABSTRACT

The present invention provides the crystal structure of BACE-1 complexed with both an active site inhibitor and an exosite peptide containing the core sequence YPYFI. The crystal structure shows the exosite peptide bound to the BACE-1 exosite and identifies a novel peptide binding site on the surface of the BACE-1. The invention also provides the residues that make up the exosite binding site within 6.0 Å of any exosite peptide atom which are: E316, K317, F318, P319, F322, G325, E326, Q327, L328, V329, C330, W331, Q332, A333, T335, D372, V373, A374, S376, D378, D379, C380, Y381.

The present patent application claims the benefit of U.S. patent application Ser. No. 10/685,898, filed on Oct. 15, 2003; and U.S. Provisional Patent Application Ser. No. 60/418,679, filed Oct. 15, 2002, the disclosure of which is incorporated herein by reference in its entirety.

### FIELD OF THE INVENTION

The present invention relates to peptides that bind to beta secretase (“β-secretase”) at a newly discovered exosite within the catalytic domain of the enzyme, and use of these peptides and variants thereof to identify therapeutic molecules useful for the treatment of neurological disorders. The present invention also relates to the identification of a crystal structure of BACE-1 complexed with both an active site inhibitor and an exosite peptide containing the core sequence YPYFI (SEQ ID NO:2). The present invention also identifies a novel peptide binding site on the surface of the BACE-1.

### BACKGROUND OF THE INVENTION

Alzheimer's disease (“AD”) is a devastating neurodegenerative disease that affects millions of elderly patients worldwide. AD is characterized clinically by progressive loss of memory, orientation, cognitive function, judgement and emotional stability. With increasing age, the risk of developing AD increases exponentially, so that by age 85 some 20-40% of the population is affected. Memory and cognitive function deteriorate rapidly within the first 5 years after diagnosis of mild to moderate impairment, and death due to disease complications is an inevitable outcome. AD is the most common cause of nursing home admittance in the United States; hence, in addition to the morbidity and mortality experienced by the patient, there are considerable economic and emotional burdens placed on the family, caregivers and society at large. The only recognized treatment currently available for AD is acetylcholinesterase inhibitors, which merely treat the symptoms of cognitive impairment. No method for prevention or treatment of the pathophysiology of AD is currently available.

Diagnosis of AD is based mainly on subjective assessments of memory and cognitive function. Definitive diagnosis can only be made post-mortem, based on histopathological examination of brain tissue from the patient. Two histological hallmarks of AD are the occurrence of neurofibrillar tangles of hyperphosphorylated tau protein and of proteinaceous amyloid plaques, both within the cerebral cortex of AD patients. The amyloid plaques are composed mainly of a peptide of 39 to 42 amino acids designated beta-amyloid, also referred to as β-amyloid, amyloid beta, Aβ, βAP, β/A4; and referred to herein as beta-amyloid and Aβ. It is now clear that the Aβ peptide is derived from a type 1 integral membrane protein, termed beta amyloid precursor protein (also referred to as “β-APP” and “APP”) through two sequential proteolytic events. First, the APP is hydrolyzed at a site N-terminal of the transmembrane alpha helix by a specific proteolytic enzyme referred to as β-secretase. The soluble N-terminal product of this cleavage event diffuses away from the membrane, leaving behind the membrane-associate C-terminal cleavage product, referred to as C99. The protein C99 is then further hydrolyzed within the transmembrane alpha helix by a specific proteolytic enzyme referred to as γ-secretase. This second cleavage event liberates the Aβ peptide and leaves a membrane-associated “stub”. The Aβ peptide thus generated is secreted from the cell into the extracellular matrix where it eventually forms the amyloid plaques associated with AD.

Several lines of evidence suggest that abnormal accumulation of Aβ plays a key role in the pathogenesis of AD. First, Aβ is the major protein component of amyloid plaques. Second, Aβ is neurotoxic and may be causally linked to the neuronal death associated with AD. Third, missense DNA mutations at several positions within the APP protein can be found in affected members but not unaffected members of several families with a genetically determined (familial) form of AD. For example, one familial form of AD is linked to a pair of mutations, referred to as the “Swedish mutations”, that are immediately proximal to the site of β-secretase-mediated hydrolysis of APP (Mullan et al., (1992) Nature Genet. 1:345-347). Patients bearing the Swedish mutant form of APP develop AD at a much earlier age (typically within the fourth decade of life) and likewise progress to severe dementia at a much earlier age. Histopathological examination of the brains of patients suffering from the “Swedish mutant” form of familial AD is identical to that of brains from patients suffering from non-familial, sporadic forms of the disease. It is therefore hypothesized that halting the production of Aβ will prevent and/or reduce the neurodegeneration and other pathologies of AD. One method of halting Aβ production would be to administer specific inhibitors of one or both of the proteolytic enzymes involved in APP processing, namely, β-secretase and γ-secretase. The molecular identity of the protein responsible for γ-secretase activity has not yet been determined, although there is a preponderance of data suggesting a role for the proteins presenilin-1 and presenilin-2 in this enzymatic action. Nevertheless, compounds that inhibit the action of γ-secretase, and thus inhibit Aβ production in cell culture have been identified by several groups.

Recently the molecular identity of the protein responsible for β-secretase activity has been determined and this protein is commonly referred to as BACE (for Beta-site APP Cleaving Enzyme). This enzyme is a type 1 membrane protein that folds into an extra-membranous globular catalytic domain that is tethered to the membrane by a single alpha helix. The catalytic domain of BACE contains the canonical signature motifs for an aspartyl protease, and the enzymatic activity of recombinant versions of the catalytic domain of human BACE is consistent with this designation. It is well known that aspartyl proteases can be effectively inhibited by small molecules and peptides that bind to, and hence block, the site on the enzyme molecule at which the chemical transformations of the substrate molecule takes place. This site of chemical reactivity is commonly referred to as the enzyme active site. For aspartyl proteases this site contains the two chemically reactive aspartic acid residues from which this class of enzymes derive its name. During the course of enzymatic action on the substrate molecule, the enzyme goes through an intermediate state in which the carbonyl carbon of the hydrolyzable amide bond of the substrate forms four coordinate bonds, engaging the active site aspartic acid residues of the enzyme.

A common strategy for inhibiting aspartyl proteases is to prepare a small peptide of amino acid composition similar to the substrate molecule but replacing the hydrolyzable amide bond with a chemical group that mimics the four coordinate carbon intermediate species just described. It is well known that chemical groups such as statines, hydroxyethylenes, hydroxyethylamines and similar structures are very effective for this purpose. Indeed, peptidic inhibitors of BACE, incorporating statine and hydroxyethylene structures have been reported. Recently the 3-dimensional structure of the catalytic domain of human BACE in complex with a hydroxyethylene-based peptidic inhibitor referred to as OM99-2 has been solved by the methods of x-ray crystallography. The resulting structure confirmed that the inhibitor binds within the enzyme active site, engaging the active site aspartic acid residues as expected. Hence, active site-directed inhibitors of BACE can be designed and may prove useful as pharmacological agents for the treatment of AD. Historically, however, it has proved difficult to develop molecules of pharmacological utility based on active site-directed inhibitors of aspartyl proteases. While very potent inhibitors have been identified in vitro, active site-directed inhibitors of aspartyl proteases may present in vivo issues of oral bioavailability and pharmacokinetic half-life.

In addition to the active site, some proteolytic enzymes contain additional binding pockets that engage the substrate protein at locations distal to the site of chemical transformation. These binding pockets are referred to as exosites and can contribute significantly to the stabilization of the enzyme-substrate binary complex by providing important structural determinants of interaction. Additionally, exosites on some proteolytic enzymes can act as allosteric regulators of enzyme activity, so that binding interactions at the exosite are transmitted through conformational changes of the enzyme to the active site, where structural changes can augment or diminish the chemical reactivity of the active site. In some cases molecules have been identified that bind to an exosite, rather than the active site, of proteolytic enzymes and these have proved to be effective inhibitors of enzymatic action. Hence, exosites represent an alternative target for inhibitory ligand binding to proteolytic enzymes. Because the exosites are distinct from the active sites of these enzymes, the nature of the molecules that bind to the exosites can be very different from active site-directed inhibitors. In addition, complete inhibition of BACE may be undesirable with respect to its other potential functions. Also, exosites often do not permit 100% inhibition. In favorable cases, the nature of the molecules binding to the exosites are more pharmacologically tractable relative to the active site-directed inhibitors of the same enzyme.

### SUMMARY OF THE INVENTION

The present invention provides peptides that specifically bind to BACE at a newly discovered exosite within the catalytic domain of the enzyme, and are referred to herein as “exosite binding peptides” or “EBPs”. The peptides of the present invention can be used to modulate BACE activity and interfere with hydrolysis of APP and APP-derived substrates.

The invention also provides methods for identifying peptides that bind to a BACE exosite comprising contacting BACE with at least one peptide, and determining whether the peptide specifically binds to BACE at a site other than the active site of BACE.

The invention also provides an isolated peptide that is capable of specifically binding to a BACE exosite peptide binding site, wherein said BACE exosite binding site comprises amino acid residues that are within 6.0 Å of each of exosite peptide atoms E316, K317, F318, P319, F322, G325, E326, Q327, L328, V329, C330, W331, Q332, A333, T335, D372, V373, A374, S376, D378, D379, C380 and Y381 (SEQ ID NO:113).

The invention also provides an isolated peptide that is capable of specifically binding to a BACE exosite peptide binding site, wherein said BACE exosite binding site comprises amino acid residues that are within 6.0 Å of any exosite peptide atom E316, K317, F318, P319, F322, G325, E326, Q327, L328, V329, C330, W331, Q332, A333, T335, D372, V373, A374, S376, D378, D379, C380, Y381 (SEQ ID NO:113).

In one embodiment, the isolated peptide comprises an amino acid sequence motif selected from the group consisting of YPYF (SEQ ID NO: 1), XYPYF (SEQ ID NO:3), XYPYFX (SEQ ID NO:4), XYPYFXX (SEQ ID NO:5), YPYFX (SEQ ID NO:6) YPYFXX (SEQ ID NO:7), HYPYF (SEQ ID NO:8), YPYFI (SEQ ID NO:2), YPYFIP (SEQ ID NO:9), YPYFIPL (SEQ ID NO:10), YPYFLPI (SEQ ID NO:11), YPYFXPI (SEQ ID NO:12), YPYFXPX (SEQ ID NO:13), HYPYFIP (SEQ ID NO:14) YPYFL (SEQ ID NO:15), YPYFLP (SEQ ID NO:16), HYPYFLP (SEQ ID NO:17), HYPYFIPL (SEQ ID NO:18), LTTYPYFIPLP (SEQ ID NO:44); TTYPYFIPLP (SEQ ID NO:45), TYPYFIPLP (SEQ ID NO:46), NLTTYPYFIPL (SEQ ID NO:48), YPYFIAL (SEQ ID NO:49), YPYFIPA (SEQ ID NO:50) YPYFIPB (SEQ ID NO:52), HYPYFI (SEQ ID NO:54), NLTTYPYFIPLP (SEQ ID NO:19) ALYPYFLPISAK (SEQ ID NO:20), WPXFI (SEQ ID NO:21), ETWPRFIPYHALTQQTLKHQQHT (SEQ ID NO:22), TAEYESRTARTAPPAPTQHWPFFIRST (SEQ ID NO:23), HWPPFFIRS (SEQ ID NO:57), YPBFIPL (SEQ ID NO:51) and YPYFIP (SEQ ID NO:10), wherein X is a naturally or nonnaturally occurring amino acid residue, and wherein B is a benzophenone group. In another embodiment, one or more amino acid residues of the amino acid sequence motif is replaced with a conservative amino acid substitution. In another embodiment, the isolated peptide consists of 5 to 30 amino acids. In another embodiment, the isolated peptide contains one or more modified amino acid residues. In another embodiment, the isolated peptide contains a label selected from the group consisting of a fluorescent label, a chromophore label, a radiolabel and a biotin label. In another embodiment, the isolated peptide comprises a terminal modification that enhances the resistance of the peptide to proteolysis. In another embodiment, the isolated peptide is cyclic. In another embodiment, the peptide is a macrocyclized peptide or a variant, such as a disulfide-cyclized peptide.

The invention further provides a composition comprising a peptide of the invention and a pharmaceutically acceptable carrier.

The invention further provides methods of using the peptides and variants thereof for identifying compounds that bind to BACE exosites and modulate BACE activity. In another aspect, the invention provides methods for treating or preventing neurological disorders such as Alzheimer's disease by administering compounds that bind to a BACE exosite and inhibit beta-amyloid production.

The invention further provides the crystal structure of BACE-1 complexed with both an active site inhibitor and an exosite peptide containing the core sequence YPYFI.

The invention further provides the residues that make up the exosite binding site within 6.0 Å of any exosite peptide atom. The invention further provides the residues that make up the exosite binding site within 6.0 Å of each of exosite peptide atoms.

The invention further provides a method of identifying a peptide that specifically binds to a BACE exosite binding site comprising: (a) contacting BACE with at least one peptide; and (b) determining whether the peptide specifically binds to the BACE exosite binding site, wherein said BACE exosite binding site comprises amino acid residues that are within 6.0 Å of each of exosite peptide atoms E316, K317, F318, P319, F322, G325, E326, Q327, L328, V329, C330, W331, Q332, A333, T335, D372, V373, A374, S376, D378, D379, C380 and Y381 (SEQ ID NO:113).

The invention further provides a method of identifying a peptide that specifically binds to a BACE exosite binding site comprising: (a) contacting BACE with at least one peptide; and (b) determining whether the peptide specifically binds to the BACE exosite binding site, wherein said BACE exosite binding site comprises amino acid residues that are within 6.0 Å of any exosite peptide atom E316, K317, F318, P319, F322, G325, E326, Q327, L328, V329, C330, W331, Q332, A333, T335, D372, V373, A374, S376, D378, D379, C380, Y381 (SEQ ID NO:113).

The invention further provides a method of identifying a modulator of BACE activity comprising the steps of (a) contacting a candidate modulator of BACE and a BACE exosite binding peptide in the presence of BACE or a BACE variant including at least one BACE exosite binding site wherein the BACE exosite binding site comprises an amino acid sequence comprising E316, K317, F318, P319, F322, G325, E326, Q327, L328, V329, C330, W331, Q332, A333, T335, D372, V373, A374, S376, D378, D379, C380, Y381 (SEQ ID NO:113) wherein amino acid numbering is based on RefSeq NP_{—}036236, and (b) determining whether there is an increase or a decrease in binding of the exosite binding peptide to BACE in the presence of the candidate BACE modulator compared to binding of the exosite binding peptide to BACE in the absence of the candidate modulator.

The invention further provides a method of identifying a therapeutic for treating a disorder involving APP processing and beta-amyloid production comprising: (a) contacting BACE with a candidate BACE exosite binding compound; and (b) determining an amount of inhibition of APP processing and beta-amyloid production.

The invention further provides a method of identifying a BACE exosite binding compound that inhibits beta-amyloid production comprising: (a) contacting a candidate exosite binding compound with a cell that expresses a beta-amyloid precursor protein and BACE, wherein the cell is capable of secreting beta-amyloid protein in the absence of the candidate exosite binding compound; and (b) determining whether the candidate exosite binding compound reduces the amount of beta-amyloid protein secreted by the cell in the absence of the candidate exosite binding compound. In one embodiment, the BACE exosite comprises amino acid residues that are within 6.0 Å of each of exosite peptide atoms E316, K317, F318, P319, F322, G325, E326, Q327, L328, V329, C330, W331, Q332, A333, T335, D372, V373, A374, S376, D378, D379, C380 and Y381 (SEQ ID NO:113). In another embodiment, the BACE exosite comprises amino acid residues that are within 6.0 Å of any exosite peptide atom E316, K317, F318, P319, F322, G325, E326, Q327, L328, V329, C330, W331, Q332, A333, T335, D372, V373, A374, S376, D378, D379, C380, Y381 (SEQ ID NO:113).

The invention further provides a method of treating a neurological disorder comprising administering to a patient in need of such treatment a therapeutically effective amount of a compound, or a pharmaceutically acceptable salt or prodrug form thereof, wherein the compound specifically binds to a BACE exosite binding site, wherein said BACE exosite binding site comprises amino acid residues that are within 6.0 Å of each of exosite peptide atoms E316, K317, F318, P319, F322, G325, E326, Q327, L328, V329, C330, W331, Q332, A333, T335, D372, V373, A374, S376, D378, D379, C380 and Y381 (SEQ ID NO:113).

The invention further provides a method of treating a neurological disorder comprising administering to a patient in need of such treatment a therapeutically effective amount of a compound, or a pharmaceutically acceptable salt or prodrug form thereof, wherein the compound specifically binds to a BACE exosite binding site, wherein said BACE exosite binding site comprises amino acid residues that are within 6.0 Å of any exosite peptide atom E316, K317, F318, P319, F322, G325, E326, Q327, L328, V329, C330, W331, Q332, A333, T335, D372, V373, A374, S376, D378, D379, C380, Y381 (SEQ ID NO:113).

In one embodiment, BACE is selected from the group consisting of an isolated BACE, an isolated BACE variant, a recombinant BACE, a recombinant BACE variant, and a BACE fusion protein. In another embodiment, the exosite binding peptide contains a label selected from the group consisting of a fluorescent label, a chromophore label, a radiolabel and a biotin label. In another embodiment, the peptide is cyclic. In another embodiment, the peptide is a macrocyclized peptide or a variant, such as a disulfide-cyclized peptide. This invention also provides a compound identified by this method, optionally comprising a pharmaceutically acceptable carrier.

In another embodiment, the invention provides a method of identifying a modulator of BACE which comprises: (a) providing the structural coordinates of a BACE exosite binding site provided in one of Tables 7-8 defining a three-dimensional structure of a BACE exosite binding site; (b) using the three-dimensional structure to design or select a test compound by computer modeling; (c) synthesizing or acquiring the test compound; and (d) contacting the test compound with BACE to determine the ability of the test compound to modulate a biological activity of BACE. In one embodiment, the biological activity is APP processing or beta-amyloid production.

The invention further provides a crystalline form comprising a BACE polypeptide wherein said BACE polypeptide comprises a BACE exosite binding site. In one embodiment, the crystalline form has lattice parameters of a=60.2 Å, b=130.6 Å, c=64.1 Å, α=γ=90°, β=91.8° and a unit cell variability of 30% in all dimensions. In another embodiment, the crystalline form has lattice parameters of a=86.5 Å, b=93.9 Å, c=131.7 Å, α=β=γ=90° and a unit cell variability of 30% in all dimensions. In a further embodiment, the crystalline form has symmetry consistent with a monoclinic space group P2₁. In a further embodiment, the crystalline from comprises a structure defined by all or a portion of the coordinates of Table 7 ±a root mean square deviation from the Cα atoms of less than 0.5 Å. In a further embodiment, the crystalline from comprises a structure defined by all or a portion of the coordinates of Table 8 ±a root mean square deviation from the Cα atoms of less than 0.5 Å. In a further embodiment, the crystalline form has symmetry consistent with the space group P2₁2₁2₁.

### BRIEF DESCRIPTION OF THE DRAWINGS

FIG. 1 shows isothermal calorimetry data quantitatively determining the binding affinity of peptide NLTTYPYFIPLP (SEQ ID NO:19) to BACE at 25° C., in Dulbecco's PBS wherein the parameters were K_{A}=1.65×10⁷ M⁻¹; K_{d}=61 nM; n=1.03; and ΔH=−12.9 kcal/mol.

FIG. 2 shows isothermal calorimetry data quantitatively determining the binding affinity of peptide ALYPYFLPISAK (SEQ ID NO:20) to BACE at 25° C. in Dulbecco's PBS wherein the parameters were K_{A}=8.86×10⁶ M⁻¹; K_{d}=113 nM; n=1.06; and ΔH=−11.8 kcal/mol

FIG. 3 shows integrated isothermal calorimetry data quantitatively determining the binding affinities of BACE-OM99-2 titrated with peptide NLTTYPYFIPLP (SEQ ID NO:19) in pH 5.3 buffer at 37° C.

FIG. 4 shows EBPs labeled with the fluorescent group Alexa488 at different positions (Molecules X, Yn and Z of Example 9) and with linkers of different lengths (Yn, wherein n=1-4).

FIG. 5 shows fluorescence anisotropy data demonstrating that labeled EBP Molecule X binds to BACE at pH 7.1 (upper panel) and pH 4.5 (lower panel). The solid lines represent fitting the data to a 1:1 binding model.

FIG. 6 shows that BMS-561871, peptide NLTTYPYFIPLP (SEQ ID NO:19) (Molecule X without the Alexa488 label) competes with the binding of Molecule X at pH 7.1 (upper panel) and pH 4.5 (lower panel), as monitored by fluorescence anisotropy.

FIG. 7 shows inhibition of the binding of Molecule X to BACE by a collection of N-terminally (upper panel) and C-terminally (lower panel) truncated unlabeled peptides monitored by fluorescence anisotropy. The numbered identifiers on the X-axis refer to the amino acid composition with respect to BMS-561871.

FIG. 8 shows the binding of Molecule Y1 to BACE at pH 4.5 by fluorescence anisotropy. The solid line represents fitting the data to a 1:1 binding model.

FIG. 9 shows that BMS-593925, peptide YPYFIPL (SEQ ID NO:10) (Molecule Y1 without the Alexa488 label) competes with the binding of Molecule Y1 at pH 4.5 monitored by fluorescence anisotropy.

FIG. 10 shows the fluorescence anisotropy measurement of Molecule Y1 binding to BACE both in the absence (circles) and in the presence (squares) of OM99-2, an active site-directed inhibitor of BACE, suggesting that Molecule Y1 does not bind to BACE at the active site.

FIG. 11 shows the inhibition of the binding of Molecule Y1 to BACE by a collection of mutated peptides based on peptide YPYFIPL (SEQ ID NO:10) (wt sequence, corresponding to 5-11 in the original NLTTYPYFIPLP peptide; SEQ ID NO:19): Upper panel: Peptides with a mutation to Ala at the indicated position. Lower panel: Peptides with a mutation to benzophenone (Bpa or B) at the indicated position. N-B and C-B represent peptides that have the Bpa group attached to the N- and C-terminus, respectively. Peptides P6B, 19B, and C-B (indicated by asterisks in the figure) have limited solubility, therefore, the inhibition of binding between Molecule B1 and BACE is under determined in these cases.

FIG. 12 shows a fluoroimage (excitation at 488 nm, emission at 530 nm) of the time course of the photo-crosslinking of BACE (2 μM) to a Bpa-containing EBP, BMS-607641, with the sequence of YPYFIPB-Alexa488 (2 μM) (SEQ ID NO:108) in the presence of 100 μM of a scrambled peptide (BMS-599271, LYPPYIF; SEQ ID NO:53) at 4° C.

FIG. 13 shows that Molecule Y3 inhibits the proteolytic activity of BACE monitored by HPLC analysis. The solid line represents fitting of the data to the Langmuir isotherm equation (Copeland, R. A., Enzymes: A Practical Introduction to Structure, Mechanism, and Data Analysis, (2^{nd} ed), Wiley-VCH, New York, N.Y. (2000)).

FIG. 14 shows integrated and fitted binding data for BACE and BMS-655507 (Ac-His-Trp-Pro-Phe-Phe-Ile-Arg-Ser; SEQ ID NO:57), at 25° C., in Dulbecco's PBS; parameters: K_{A}=1.09×10⁶ M⁻¹; K_{d}=0.914 μM; n=0.71; ΔH=−18.04 kcal/mol.

FIG. 15 shows integrated and fitted binding data for BACE and BMS-655507 (Ac-His-Trp-Pro-Phe-Phe-Ile-Arg-Ser; SEQ ID NO:57), at 25° C., in 50 mM NaOAc, pH 4.5; parameters: K_{A}=6.10×10⁵M⁻¹; K_{d}=1.64 μM; n=0.86; ΔH=−13.35 kcal/mol.

FIG. 16 shows the BACE-1 nucleotide amino acid sequences (GenBank Accession No. NP_{—}036236) of the two constructs used for x-ray structure determination. FIG. 16A represents the Construct #1 nucleotide sequence (SEQ ID NO:110) and amino acid sequence (SEQ ID NO:111) that were used for the BMS-597041 structure determination. In the amino acid sequence, the underlined amino acids represent the T7 tag, the amino acids that are boxed represent the Pro-domain, the amino acids in regular text represent the catalytic domain, and the amino acids that are in bold/italics are within the catalytic domain and represent the putative binding site residues that are within 6 Å of exosite peptide BMS-561871. FIG. 16B represents the Construct #2 nucleotide sequence (SEQ ID NO:112) and amino acid sequence (SEQ ID NO:113) that were used for the BMS-561871 structure determination. In the amino acid sequence, the underlined amino acids represent the T7 tag, the amino acids that are boxed represent the Pro-domain, the amino acids in regular text represent the catalytic domain, the amino acids that are in bold are the mutated amino acids, and the amino acids that are in bold/italics are within the catalytic domain and represent the putative binding site residues that are within 6 Å of exosite peptide BMS-561871.

FIG. 17 is a schematic representing the 2-dimensional structures of (A) active site inhibitor DPH-153979; (B) exosite peptide BMS-597041 (YPYFIPL (SEQ ID NO;10)-Alexa488 Fluorophore; and (C) exosite peptide BMS-561871 (NLTTYPYFIPLP (SEQ ID NO:19)).

FIG. 18A is a schematic representing a Cα ribbon diagram of BACE-1 (light gray) in complex with active site inhibitor DPH-153979 (light gray stick) and YPYFI (SEQ ID NO:2) of the exosite peptide BMS-597041 (dark gray stick) to illustrate the location of the exosite peptide relative to the active site inhibitor. FIG. 18B is a schematic representing a Cα ribbon diagram of BACE-1 (light gray) in complex with active site inhibitor DPH-153979 (light gray stick) and YPYFIPL (SEQ ID NO:10) of the exosite peptide BMS-561871 (dark gray stick) to illustrate the location of the exosite peptide relative to the active site inhibitor.

FIG. 19A is a surface representation of BACE-1 residues along with YPYFIPL (SEQ ID NO:10) of BMS-561871 to show the shallow groove of the exosite on the surface of the protein. Surface residues are within 5 Å of the exosite peptide shown. FIG. 19B represents a stereo view of BACE-1 residues within 3.5 Å (light gray) along with exosite peptide YPYFIPL (dark gray). Hydrogen bonds are shown as dashed lines. Amino acid numbering corresponds to the numbering scheme of Hong et al. (Hong et. al., (2000) Science, 290:150-153).

### BRIEF DESCRIPTION OF THE TABLES

Table 1 is a table showing peptides having a YPYF (SEQ ID NO: 1) motif that specifically bind to a BACE exosite.

Table 2 is a table showing peptides with other sequence motifs.

Table 3 is a table showing the calculated and fitted data for four experiments with BACE, OM99-2, and peptide #1, conducted at pH 5.3, 37° C.

Table 4 is a table showing the exosite binding peptides identified by solution panning at pH 5.2.

Table 5 is a table showing the calculated and fitted thermodynamic data for experiments with BACE and BMS-655507 conducted at pH 4.5 and 7.0, 25° C.

Table 6 is a table showing data collection and refinement statistics.

Table 7 is a table showing the coordinates of T7-BACE1(A14-T454) complexed with DPH-153979 and BMS-597041. The numbering of the residues used in this Table correspond to the numbering scheme of Hong et. al. (Hong et. al., (2000) Science, 290:150-153).

Table 8 is a table showing the coordinates of T7-BACE 1 (A114-T454/R56K/R57K) complexed with DPH-153979 and BMS-561871. The numbering of the residues used in this Table correspond to the numbering scheme of Hong et. al. (Hong et. al., (2000) Science, 290:150-153).

Table 9 is a table correlating the amino acid sequence numbering between BACE-1 GenBank Accession No. NP_{—}036236 and the numbering scheme of Hong et. al. (Hong et. al., (2000) Science, 290:150-153) for the residues within 6 Å of exosite peptide BMS-561871.

### DETAILED DESCRIPTION OF THE INVENTION

In accordance with the present invention, we have discovered peptides that specifically bind to a Beta-site APP Cleaving Enzyme (BACE) binding site that is not the BACE active site. A BACE exosite is an important target site for modulating the processing of APP and the production of Aβ.

The present invention provides isolated peptides that specifically bind to BACE at an exosite and modulate BACE activity. Peptides that specifically bind to BACE at an exosite are also referred to herein as “exosite binding peptides” (EBPs). The terms “specific binding” or “specifically bind” refer to the interaction between a protein and a binding molecule, such as a compound. The interaction is dependent upon the presence of a particular structure (i.e., an enzyme binding site, an antigenic determinant or epitope) of the protein that is recognized by the binding molecule. For example, if a compound is specific for enzyme binding site “A”, the presence of the compound in a reaction containing a protein including enzyme binding site A, and a labeled peptide that specifically binds to enzyme binding site A will reduce the amount of labeled peptide bound to the protein. In contrast, nonspecific binding of a compound to the protein does not result in a concentration-dependent displacement of the labeled peptide from the protein.

As used herein, the term “BACE exosite” refers to a BACE binding site that is not the BACE active site. Amino acid residues that define the exosite peptide binding site are within 6 Å of the BMS-561871 exosite peptide atom are: E316, K317, F318, P319, F322, G325, E326, Q327, L328, V329, C330, W331, Q332, A333, T335, D372, V373, A374, S376, D378, D379, C380, Y381 (SEQ ID NO:112) where the amino acid numbering based on GenBank Accession No. NP_{—}036236.

As used herein the term “active site” means the site on the enzyme molecule at which the chemical transformations of the substrate molecule take place.

The present invention provides a method for identifying peptides that specifically bind to a BACE exosite comprising:

(a) contacting BACE with at least one peptide; and

(b) determining whether the peptide specifically binds to BACE at a site other than the active site of BACE.

In one embodiment, the EBPs of the present invention can be used to treat disorders such as neurodegenerative disorders. In this embodiment, the EBP is administered to a patient in a therapeutic composition with a pharmaceutically acceptable carrier. Moreover, a combination of EBPs may be administered to a patient to treat a neurodegenerative disorder, such as Alzheimer's disease.

Peptides that bind to BACE exosites can be identified by screening peptide libraries. Preferably, phage display random libraries and phage ELISA assays are used to identify the EBPs. Preparation of phage display libraries and phage ELISA assays are known to those skilled in the art, see, for example, B. K. Kay, J. Winter, J. McCafferty (eds.), Phase Display of Peptides and Proteins. A Laboratory Manual, Academic Press, (1996), chapters 5, 7, 13 and 16. In a preferred embodiment, the peptides of the peptide libraries are 5 mer to 30 mer peptides. The phage display library can be screened by isolating viral particles that bind to targets. The isolates can be grown up, and the displayed peptide sequence responsible for binding can be deduced by DNA sequencing.

In a preferred embodiment, the present invention provides EBPs comprising an amino acid sequence having a Tyr-Pro-Tyr-Phe (also referred to herein as “YPYF”) (SEQ ID NO:1) motif wherein the EBPs are capable of specifically binding to a BACE exosite and inhibiting BACE activity.

Other preferred EBPs of the present invention include a BACE exosite binding motif comprising amino acid residues Tyr-Pro-Tyr-Phe-Ile (also referred to herein as “YPYFI”) (SEQ ID NO:2). Preferred EBPs of the present invention comprise at least one of the following amino acid sequences: Xaa-Tyr-Pro-Tyr-Phe (SEQ ID NO:3), Xaa-Tyr-Pro-Tyr-Phe-Xaa (SEQ ID NO:4), Xaa-Tyr-Pro-Tyr-Phe-Xaa-Xaa (SEQ ID NO:5), Tyr-Pro-Tyr-Phe-Xaa (SEQ ID NO:6) Tyr-Pro-Tyr-Phe-Xaa-Xaa (SEQ ID NO:7), His-Tyr-Pro-Tyr-Phe (SEQ ID NO:8), Tyr-Pro-Tyr-Phe-Ile (SEQ ID NO:2), Tyr-Pro-Tyr-Phe-Ile-Pro (SEQ ID NO:9), Tyr-Pro-Tyr-Phe-Ile-Pro-Leu (SEQ ID NO:10), Tyr-Pro-Tyr-Phe-Leu-Pro-Ile (SEQ ID NO:11), Tyr-Pro-Tyr-Phe-Xaa-Pro-Ile (SEQ ID NO: 12), Tyr-Pro-Tyr-Phe-Xaa-Pro-Xaa (SEQ ID NO: 13), His-Tyr-Pro-Tyr-Phe-Ile-Pro (SEQ ID NO:14) Tyr-Pro-Tyr-Phe-Leu (SEQ ID NO:15), Tyr-Pro-Tyr-Phe-Leu-Pro (SEQ ID NO: 16), His-Tyr-Pro-Tyr-Phe-Leu-Pro (SEQ ID NO: 17), and His-Tyr-Pro-Tyr-Phe-Ile-Pro-Leu (SEQ ID NO:18). As used herein the term “Xaa” means any amino acid, i.e., either naturally or non-naturally occurring amino acid.

The most preferred EBPs of the present invention are Asn-Leu-Thr-Thr-Tyr-Pro-Tyr-Phe-Ile-Pro-Leu-Pro (SEQ ID NO:19) also referred to herein as “NLTTYPYFIPLP” and “BMS-561871”; Ala-Leu-Tyr-Pro-Tyr-Phe-Leu-Pro-Ile-Ser-Ala-Lys (SEQ ID NO:20) also referred to herein as “ALYPYFLPISAK” and “BMS-561877”; and Tyr-Pro-Tyr-Phe-Ile-Pro-Leu (SEQ ID NO:10) also referred to herein as “YPYFIPL” and “BMS-593925.”

Other preferred EBPs of the present invention comprise amino acid sequences having a WPXFI (SEQ ID NO:21) motif. Preferred EBPs having the WPXFI motif are Glu-Thr-Trp-Pro-Arg-Phe-Ile-Pro-Tyr-His-Ala-Leu-Thr-Gln-Gln-Thr-Leu-Lys-His-Gln-Gln-His-Thr (SEQ ID NO:22), Thr-Ala-Glu-Tyr-Glu-Ser-Arg-Thr-Ala-Arg-Thr-Ala-Pro-Pro-Ala-Pro-Thr-Gln-His-Trp-Pro-Phe-Phe-Ile-Arg-Ser-Thr (SEQ ID NO:23) and His-Trp-Pro-Phe-Phe-Ile-Arg-Ser (SEQ ID NO:57).

In the most preferred embodiment, the EBPs of the present invention contain from about 5 to about 30 amino acid residues.

The amino acid sequence of the subject EBPs can be modified for such purposes as enhancing therapeutic or prophylactic efficacy, or stability (e.g., ex vivo shelf life and resistance to proteolytic degradation in vivo). Such modified peptide can be produced, for instance, by amino acid substitution, deletion, or addition different codon usage. Likewise, different codons may be selected to increase the rate at which expression of the peptide/polypeptide occurs in a particular prokaryotic or eukaryotic host in accordance with the frequency with which particular codons are utilized by the host.

Variant EBPs resulting from amino acid substitutions, deletions, or additions of the EBP sequences described herein are within the scope of the present invention. Examples of such variant EBPs are EBPs wherein a leucine is replaced with an isoleucine or valine, an aspartic acid with a glutamic acid, a threonine with a serine, or a similar replacement of an amino acid with a structurally related amino acid (i.e., conservative mutations). Conservative replacements are those that take place within a family of amino acids that are related in their side chains. Genetically encoded amino acids can be divided into the following families: (1) acidic: aspartatic acid, glutamatic acid; (2) basic: lysine, arginine, histidine; (3) nonpolar: alanine, valine, leucine, isoleucine, proline, phenylalanine, methionine, tryptophan; (4) uncharged polar: glycine, asparagine, glutamine, cysteine, serine, threonine, and tyrosine; (5) aromatic: phenylalanine, tryptophan, and tyrosine; (6) aliphatic: glycine, alanine, valine, leucine, isoleucine, serine, threonine, with serine and threonine optionally being grouped separately as aliphatic-hydroxyl; and (7) amide: asparagine, glutamine; and (8) sulfur-containing: cysteine and methionine (see, for example, Stryer (ed.), Biochemistry, (2^{nd} ed.), WH Freeman and Co. (1981)).

The EBPs of the present invention can also be peptide mimics wherein one or more of the amino acid residues is replaced with a nonnaturally occurring amino acid residue. For example, one or more amino acid residues may be tagged with a photoaffinity label such as, for example, benzophenone.

Those skilled in the art of peptide chemistry are aware that amino acid residues occur as both D and L isomers, and that the instant invention contemplates the use of either D or L isomers or a mixture of isomers of amino acid residues incorporated in the synthesis of the peptides described herein.

The EBPs of the present invention can be produced by conventional methods known to those skilled in the art. In one embodiment, the peptide may be produced by expression from a transformed host. For example, a host cell transfected with a nucleic acid vector directing expression of a nucleotide sequence encoding the EBP can be cultured under appropriate conditions to allow expression of the peptide to occur. The peptide may be secreted and isolated from a mixture of cells and medium containing the recombinant EBP. Alternatively, the peptide may be retained cytoplasmically and the cells harvested, lysed and the peptide isolated. A cell culture includes host cells, media and other byproducts. Suitable media for cell culture are well known in the art. The recombinant EBP can be isolated from cell culture medium, host cells, or both using techniques known in the art for purifying peptides including ion-exchange chromatography, reverse phase chromatography, gel filtration chromatography, ultrafiltration, electrophoresis, and immunoaffinity purification with antibodies specific for such peptides. In one embodiment of the invention, the recombinant EBP is a fusion protein containing a domain that facilitates its purification, such as EBP-GST fusion protein.

In addition, cell-free translation systems (see Sambrook et al., Molecular Cloning: A Laboratory Manual, (2^{nd} ed.) Cold Spring Harbor Laboratory, Cold Spring Harbor, N.Y., (1989)) can be used to produce recombinant EBPs. Suitable cell-free expression systems for use in accordance with the present invention include rabbit reticulocyte lysate, wheat germ extract, canine pancreatic microsomal membranes, E. coli S30 extract, and coupled transcription/translation systems (Promega Corp., Madison, Wis.). These systems allow expression of recombinant polypeptides or peptides upon the addition of cloning vectors, DNA fragments, or RNA sequences containing coding regions and appropriate promoter elements.

In another embodiment, nucleic acid sequences encoding the EBPs of the present invention may be synthesized, in whole or in part, using chemical methods well known in the art (see, e.g., Caruthers, M. H. et al., (1980) Nucl. Acids Res. Symp. Ser. 215-223; Horn, T. et al., (1980) Nucl. Acids Res. Symp. Ser. 225-232). Such nucleic acid sequences can be expressed by conventional methods known to those skilled in the art. The present invention provides isolated codon-usage variants that do not alter the polypeptide sequence or biological activity of the EBPs disclosed herein. For example, a number of amino acids are designated by more than one triplet. Codons that specify the same amino acid, or synonyms may occur due to degeneracy in the genetic code. Examples include nucleotide codons CGT, CGG, CGC, and CGA encoding the amino acid, arginine (R); or codons GAT, and GAC encoding the amino acid, aspartic acid (D). Thus, a protein or peptide can be encoded by one or more nucleic acid molecules that differ in their specific nucleotide sequence, but still encode peptide or protein molecules having identical sequences. The amino acid coding sequence is as follows:

| | |
|---|---|
| Three | One |
| Letter | Letter |

| | | | |
|---|---|---|---|
| Amino Acid | Symbol | Symbol | Codons |

| | | | |
|---|---|---|---|
| Alanine | Ala | A | GCU, GCC, GCA, GCG |
| Cysteine | Cys | C | UGU, UGC |
| Aspartic Acid | Asp | D | GAU, GAC |
| Glutamic Acid | Glu | E | GAA, GAG |
| Phenylalanine | Phe | F | UUU, UUC |
| Glycine | Gly | G | GGU, GGC, GGA, GGG |
| Histidine | His | H | CAU, CAC |
| Isoleucine | Ile | I | AUU, AUC, AUA |
| Lysine | Lys | K | AAA, AAG |
| Leucine | Leu | L | UUA, UUG, CUU, CUC, CUA, |
| | | | CUG |
| Methionine | Met | M | AUG |
| Asparagine | Asn | N | AAU, AAC |
| Proline | Pro | P | CCU, CCC, CCA, CCG |
| Glutamine | Gln | Q | CAA, CAG |
| Arginine | Arg | R | CGU, CGC, CGA, CGG, AGA, |
| | | | AGG |
| Serine | Ser | S | UCU, UCC, UCA, UCG, AGU, |
| | | | AGC |
| Threonine | Thr | T | ACU, ACC, ACA, ACG |
| Valine | Val | V | GUU, GUC, GUA, GUG |
| Tryptophan | Trp | W | UGG |
| Tyrosine | Tyr | Y | UAU, UAC |

The codon-usage variants may be generated by recombinant DNA technology. Codons may be selected to optimize the level of production of the EBP in a particular prokaryotic or eukaryotic expression host, in accordance with the frequency of codon utilized by the host cell. Alternative reasons for altering the nucleotide sequence encoding an EBP include the production of RNA transcripts having more desirable properties, such as an extended half-life or increased stability. A multitude of variant nucleotide sequences that encode the respective EBPs may be isolated, as a result of the degeneracy of the genetic code. Accordingly, the present invention provides selecting every possible triplet codon to generate every possible combination of nucleotide sequences that encode the disclosed EBPs.

Alternatively, the peptide or protein itself may be produced using chemical methods to synthesize the amino acid sequence of the EBP, or a portion thereof. For example, peptide synthesis can be performed using various solid-phase techniques (see, e.g., Roberge, J. Y. et al., (1995) Science 269:202-204), cleavage from a naturally-derived, synthetic or semi-synthetic polypeptide, automated synthesis using a peptide synthesizer, or a combination of these techniques.

Solid-phase techniques that can be used to synthesize the EBPs of the present invention are described in G. Barany and R. B. Merrifield, The Peptides: Analysis, Synthesis, Biology; Volume 2-“Special Methods in Peptide Synthesis, Part A”, pp. 3-284, (E. Gross and J. Meienhofer, eds.), Academic Press, New York, 1980; and in J. M. Stewart and J. D. Young, Solid-Phase Peptide Synthesis, 2^{nd} Ed., Pierce Chemical Co., Rockford, Ill., (1984), for example. The preferred strategy for use in this invention is based on the Fmoc (9-Fluorenylmethylmethyloxycarbonyl) group for temporary protection of the α-amino group, in combination with the tert-butyl group for temporary protection of the amino acid side chains (see for example E. Atherton and R. C. Sheppard, “The Fluorenylmethoxycarbonyl Amino Protecting Group”, in The Peptides: Analysis, Synthesis, Biology; Volume 9-“Special Methods in Peptide Synthesis, Part C”, pp. 1-38, (S. Undenfriend and J. Meienhofer, eds.), Academic Press, San Diego, (1987)).

The peptides are synthesized in a stepwise manner on an insoluble polymer support (also referred to as “resin”) starting from the C-terminus of the peptide. A synthesis is begun by appending the C-terminal amino acid of the peptide to the resin through formation of an amide linkage. This allows the eventual release of the resulting peptide as a C-terminal amide. The C-terminal amino acid and all other amino acids used in the synthesis are required to have their α-amino groups and side chain functionalities (if present) differentially protected such that the α-amino protecting group may be selectively removed during the synthesis. The coupling of an amino acid is performed by activation of its carboxyl group as an active ester and reaction thereof with the unblocked α-amino group of the N-terminal amino acid appended to the resin. The sequence of α-amino group deprotection and coupling is repeated until the entire peptide sequence is assembled. The peptide is then released from the resin with concomitant deprotection of the side chain functionalities, usually in the presence of scavengers to limit side reactions. The resulting peptide is finally purified by reverse phase HPLC.

The synthesis of the peptidyl-resins required as precursors to the final EBP peptides utilize commercially available cross-linked polystyrene polymer resins. Preferred for use in this invention is 4-(2′,4′-dimethoxyphenyl-Fmoc-aminomethyl)-phenoxyacetyl-p-methyl benzhydrylamine resin (Rink amide MBHA resin), Novabiochem, San Diego, Calif. Coupling of amino acids can be accomplished using HOBT or HOAT active esters produced from HBTU/HOBT in the presence of a tertiary amine such as DIEA, or from DIC/HOAT, respectively.

Preferred Fmoc amino acids for use in synthesizing the EBPs of the present invention are the derivatives shown below.

### Orthogonally Protected Amino Acids used in Solid Phase Synthesis

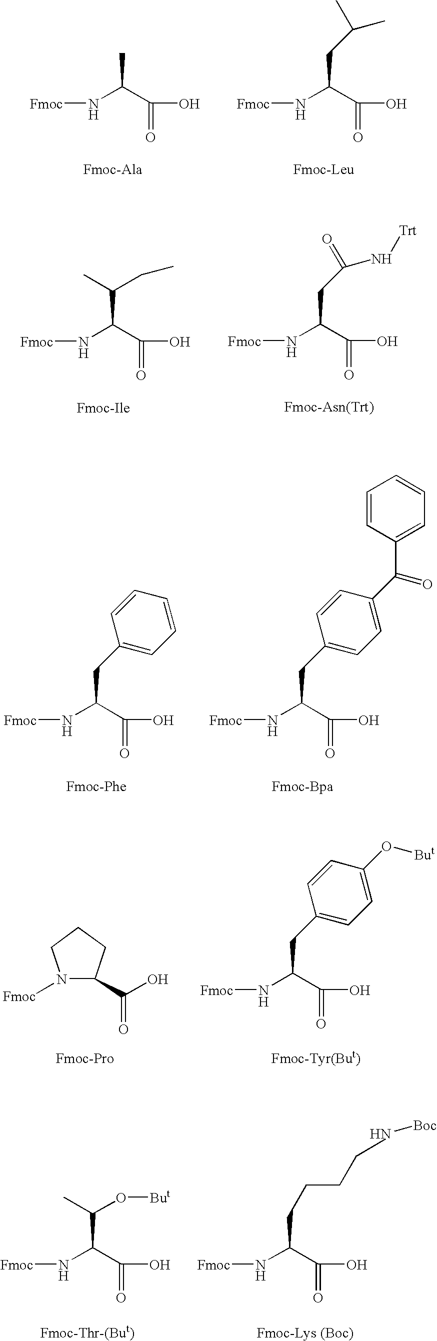

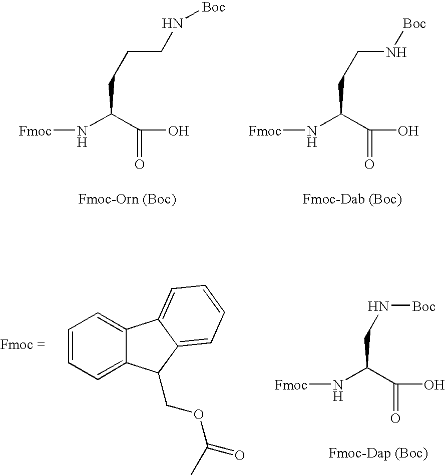

The peptidyl-resin precursors for their respective peptides may be cleaved and deprotected using any of the standard procedures described in the literature (see, for example, King et al., (1990) Int. J. Peptide Protein Res. 36:255-266). A preferred method for use in this invention is the use of TFA in the presence of water and TIS as scavengers. Typically, the peptidyl-resin is stirred in TFA/water/TIS (94:3:3, v:v:v; 1 mL/100 mg of peptidyl resin) for 1.5-2 hrs at room temperature. The spent resin is then filtered off and TFA solution is concentrated or dried under reduced pressure. The resulting crude peptide is either washed with Et₂O or redissolved directly into DMSO or 50% aqueous acetic acid for purification by preparative HPLC.

Peptides with the desired purity can be obtained by purification using preparative HPLC on, for example, either a Waters Model 4000 or a Shimadzu Model LC-8A liquid chromatograph. The solution of crude peptide is injected into a YMC S5 ODS (20×100 mm) column and eluted with a linear gradient of MeCN in water, both buffered with 0.1% TFA, using a flow rate of 14-20 mL/min with effluent monitoring by UV absorbance at 220 nm. The structures of the purified peptides are typically confirmed by electro-spray MS analysis.

Attachment of a fluorescent label to the EBP peptides described herein may be accomplished by reacting either the α-amino group of the N-terminal amino acid residue of the EBP peptide or the α-amino group of the side chain of a α,ω-diamino acid appended to the C-terminus of a EBP peptide with the N-hydroxysuccinimidyl ester derivatives of the desired fluorophore. Preferred for use in this invention is the Alexa Fluor 488 fluorophore (“Alexa488”) (Molecular Probes, Eugene, Oreg.).

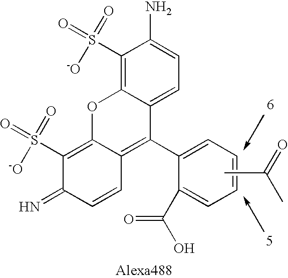

The following abbreviations are employed in the Examples and elsewhere herein:

TMS=trimethylsilyl; FMOC=fluorenylmethoxycarbonyl; Boc or BOC=tert-butoxycarbonyl; Bpa=p-benzoyl phenylalanine; HOAc or AcOH=acetic acid; MeCN=acetonitrile; DMF=N,N-dimethylformamide; TFA=trifluoroacetic acid; TIS=Triisopropylsilane; Et₂O=diethyl ether; NMP=N-methylpyrrolidone; DCM=dichloromethane; HOBT=1-hydroxybenzotriazole; HOAT=1-hydroxy-7-azabenzotriazole; HBTU=2-(1H-Benzotriazole-1-yl)-1,1,3,3-tetramethyluronium hexafluorophosphate; DIC═N,N′-diisopropylcarbodiimide; DIEA=N,N-diisopropylethylamine; min=minute(s); h or hr=hour(s); L=liter; mL=milliliter; μL=microliter; g=gram(s); mg=milligram(s); mol=mole(s); mmol=millimole(s); meq=milliequivalent; rt=room temperature; sat or sat'd=saturated; aq.=aqueous; HPLC=high performance liquid chromatography; LC/MS=high performance liquid chromatography/mass spectrometry; MS or Mass Spec=mass spectrometry.

In accordance with the present invention, isolated and/or synthetic EBPs can also be used to identify BACE exosites, and are a useful tool for characterizing the structure of BACE exosites. For example, a BACE exosite may be characterized by crosslinking an EBP tagged with a photoaffinity group or photoaffinity label to the BACE exosite. The terms “photoaffinity group” and “photoaffinity label” refer to a substituent on the inhibitor which can be activated by photolysis at an appropriate wavelength to undergo a crosslinking photochemical reaction with BACE. An example of a “photoaffinity group” is a benzophenone substituent.

In another embodiment of the present invention, the EBPs can be used as a BACE probe.

The following definitions apply to the terms used throughout this specification, unless otherwise defined in specific instances:

The term “BACE” as used herein refers to all forms of BACE, including BACE variants and proteins including the catalytic domain of BACE, or a fragment of BACE containing a BACE exosite. A representative, but non-limiting, example of BACE is a protein encoded by all or a fragment of the nucleic acid of GenBank Accession No. NM012104. A representative, but non-limiting, example of BACE is a protein of GenBank Accession No. NP_{—}036236.

“Modulator of BACE” or “BACE modulator” as used herein refers to a compound that alters the activity of BACE, such as, for example, agonists that increase the activity of BACE or antagonists that inhibit the activity of BACE.

The term “compound” as used herein includes but is not limited to small molecules, peptides, nucleic acid molecules and antibodies.

As used herein, “candidate modulator of BACE” is intended to mean any compound that can be screened for activity to inhibit BACE using the assay of the invention described herein. It is understood that a “candidate modulator of BACE”, which is active in the assay of the invention for inhibiting BACE activity, can subsequently be used as a “BACE modulator” or “BACE inhibitor”. It is also understood that a “candidate modulator of BACE”, which is active in the assay of the invention for inhibiting BACE activity, can subsequently be used in pharmaceutical compositions for the treatment of degenerative neurological disorders involving beta-amyloid production, preferably for the treatment of Alzheimer's disease.

As used herein, “candidate inhibitor of beta-amyloid production” is intended to mean any compound that can be screened for activity to inhibit the production of beta-amyloid peptide, or the proteolytic activity leading to the production of beta-amyloid peptide, using the assay of the invention described herein. It is understood that a “candidate inhibitor of beta-amyloid production”, which is active in the assay of the invention for inhibiting the production of beta-amyloid peptide, can subsequently be used as a “beta-amyloid peptide inhibitor.” It is also understood that a “candidate inhibitor of beta-amyloid production”, which is active in the assay of the invention for inhibiting the production of beta-amyloid peptide, can subsequently be used in pharmaceutical compositions for the treatment of degenerative neurological disorders involving beta-amyloid production, preferably for the treatment of Alzheimer's disease.

The “inhibitory concentration” of a BACE modulator or inhibitor is intended to mean the concentration at which a compound screened in an assay of the invention inhibits a measurable percentage of BACE activity. Examples of “inhibitory concentration” values range from IC₅₀ to IC₉₀, and are preferably, IC₅₀, IC₆₀, IC₇₀, IC₈₀, or IC₉₀, which represent 50%, 60%, 70%, 80% and 90% reduction in BACE activity, respectively. More preferably, the “inhibitory concentration” is measured as the IC₅₀ value. It is understood that another designation for IC₅₀ is the half-maximal inhibitory concentration.

Likewise, as used herein, “inhibitory concentration” of a beta-amyloid production inhibitor is intended to mean the concentration at which a compound screened in an assay of the invention inhibits a measurable percentage of beta-amyloid peptide production. Examples of “inhibitory concentration” values range from IC₅₀ to IC₉₀, and are preferably, IC₅₀, IC₆₀, IC₇₀, IC₈₀, or IC₉₀, which represent 50%, 60%, 70%, 80% and 90% reduction in beta-amyloid peptide production, respectively. More preferably, the “inhibitory concentration” is measured as the IC₅₀ value. It is understood that another designation for IC₅₀ is the half-maximal inhibitory concentration.

The EBPs of the present invention are particularly useful for identifying inhibitors of Aβ production. The EBPs can be used in competitive binding assays to identify inhibitors of proteolytic activity leading to Aβ production for the treatment of neurological disorders, such as Alzheimer's disease, Down's syndrome and other disorders involving Aβ, APP, and/or Aβ/APP associated macromolecules. Such competitive binding assays can identify compounds that interfere with the binding of EBPs to isolated BACE, complexes of BACE and other macromolecules, relevant tissues, cell lines, and membranes derived from relevant tissues and cell lines.

In one embodiment, the present invention provides a method for identifying modulators of BACE comprising the steps of:

(a) contacting a candidate modulator of BACE and an exosite binding peptide (EBP) in the presence of a BACE including at least one BACE exosite; and

(b) determining whether there is a decrease in binding of the exosite binding peptide to BACE in the presence of the candidate BACE modulator compared to binding of the exosite binding peptide to BACE in the absence of the candidate modulator.

The binding to and displacement from BACE of exosite binding peptides (EBPs) can be determined by methods well known to those skilled in the art. The form of BACE used for such experiments can be recombinant or natural full length BACE within the environment of a cellular membrane, or solubilized from a membrane by appropriate treatment with a detergent. Alternatively, the purified, recombinant catalytic domain of BACE can be used in the binding measurements. BACE molecules such as for example, allelic variants, fragments, or fusion proteins including at least one BACE exosite of interest are within the scope of the invention for use in the screening assays herein. In a preferred embodiment of the present invention BACE is recombinant human BACE catalytic domain as described in Mallender et al., (2001) Mol. Pharmacol. 59:619-626, and as described herein in Example 3.

Binding of the EBPs to BACE can be measured, for example, by methods such as isothermal titration calorimetry, nuclear magnetic resonance spectroscopy, BIAcore technology and the like. In a preferred embodiment, the EBPs can be modified by the incorporation of a chromophoric, fluorophoric or radioactive species to provide a convenient label with which to follow the interactions of the peptides with the macromolecular enzyme. As an example, a fluorescent molecule can be covalently attached to the amino terminus, to the carboxyl terminus, or to specific amino acid side chains (e.g., lysines and cysteines) of the peptide by application of standard peptide chemistry that is well known to those skilled in the art. For example, the EBP can be labeled with Alexa488 (Molecular Probes, Eugene, Oreg.). Once labeled and purified, the now fluorescent EBP can be conveniently used to measure formation of a binary complex with the BACE molecule.

In one aspect of the present invention, the fluorescent EBP can be mixed with BACE under conditions that optimally promote binding, for a sufficient time to establish an equilibrium between the bound and free forms of the enzyme and peptide. The free peptide can then be rapidly separated from the enzyme-bound population by any of several methods that effect separation of molecules based on molecular mass, such as gel filtration chromatography, dialysis and membrane filtration. The amount of fluorescent EBP associated with the enzyme can then be quantified by fluorescence spectroscopy. By measuring the concentration of EBP bound to the enzyme as a function of enzyme and EBP concentration, the equilibrium dissociation constant, K_{d}, for the enzyme-EBP binary complex can be determined by standard methods well known to those trained in the art (see, for example, Copeland, R. A., Enzymes: A Practical Introduction to Structure, Mechanism and Data Analysis, (2^{nd} ed.), Wiley-VCH, New York, N.Y. (2000)). Having determined the K_{d}, one can mix a specific concentration of BACE and EBP to establish a particular level of EBP occupancy on BACE. Addition of compounds that compete with EBPs for binding to BACE would cause a shift in the fractional occupancy of the fluorescent EBP on BACE. By measuring the shift in fractional occupancy as a function of the concentration of competing compound, one can define the K_{d} of the competing compound by methods well known to those skilled in the art (see, for example, Copeland, R. A., Enzymes: A Practical Introduction to Structure, Mechanism and Data Analysis, (2^{nd} ed.), Wiley-VCH, New York, N.Y. (2000)).

Often, the fluorescence properties of a molecule will change upon complex formation with a protein. Hence, changes in a fluorescence emission wavelength maximum or fluorescence intensity may accompany binding of the labeled EBP to BACE. In such cases, the change in fluorescence property can be used as a direct measure of binding, without the need to physically separate the bound and free populations of labeled EBP.

In a preferred embodiment, the polarization (or anisotropy) of fluorescence is measured with a suitable instrument. The degree of fluorescence polarization depends on the rotational freedom of the fluorescent molecule. When free in solution the fluorescence polarization of the labeled EBP would have a characteristic low value. Upon complexation with BACE, the rotational freedom is diminished and the degree of fluorescence polarization increases markedly. These changes in characteristic fluorescence polarization can therefore be used to measure the fractional occupancy of EBPs on BACE and, as described above, can also be used to measure the binding affinity of competing molecules. In a manner similar to that described above, a fixed concentration mixture of BACE and labeled EBP is mixed with varying concentrations of a competing compound. Displacement of the EBP caused by competition with the compound for the binding site on BACE is quantified by the changes in fluorescence polarization value.

Alternatively, a fluorescent or chromophoric molecule can be covalently associated with the BACE enzyme through standard protein chemistry methods that are well known to those skilled in the art. The spectroscopic features of the molecule are chosen to overlap those of a fluorescent group attached to the EBP as described above, such that the absorbance maximum of the species attached to the enzyme overlaps the fluorescence maximum of the species attached to the EBP. When the enzyme and EBP are separate, the maximal fluoresence of the species attached to the EBP is realized. However, when the binding of the EBP to BACE brings the spectroscopic species attached to BACE and the EBP into proximity, the overlap of spectral properties will cause a diminution of fluorescence intensity for the group attached to the EBP in what is commonly referred to as Fluorescence Resonance Energy Transfer (FRET). The diminution of fluorescence intensity that accompanies binding between BACE and the EBP can be directly quantified as a measure of binding interactions. The addition of a competing molecule to a mixture of the BACE/EBP FRET pair would cause a relief of the fluorescence intensity quenching which could thus be used to measure competitive binding of compounds to the EBP binding site on BACE.

In yet another embodiment, the EBP is labeled by incorporation of a radioactive species, such as ³H, ¹⁴C, ³⁵S, ³³P, ¹²⁵I, etc., by standard methods of peptide chemistry. In a manner similar to that described above, the binding of the radiolabeled EBP to BACE can be followed by mixing the peptide and protein together under optimal conditions and then rapidly separating the free peptide population from the enzyme-bound population.

In a further embodiment of the present invention an affinity sequence can be appended to the amino acid sequence of the BACE enzyme using standard methods of recombinant DNA technology. Examples of such affinity sequences include, but are not limited to multiple histidine residues for complexation with transition metals, epitopic sequences that are recognized by specific antibodies, and biotin which is recognized by the protein streptavidin. Technology well known to those skilled in the art commonly referred to as a Scintillation Proximity Assay (SPA) can be used to measure binding of the labeled EBP to BACE and the displacement of this binding by competing molecules.

Polymeric beads that are saturated with scintillation fluid and are chemically attached to the recognition partner of the affinity sequence, i.e., chemically attached to a transition metal, a specific antibody, or to streptavidin or other recognition partners, can be mixed with the BACE protein containing the affinity sequence to form a stable complex between the BACE protein and the polymeric bead. When radiolabeled EBP is added to this mixture, the binding of the EBP to BACE brings the radiolabel on the peptide into close proximity with the scintillation fluid incorporated into the polymeric bead. The resulting light emission from the scintillation fluid can then be quantified as a measure of binding interaction between BACE and the peptide. In a manner similar to that outlined above, the signal measured in this way can be used to quantify binding of the labeled EBP to BACE and the displacement of this binding by competing molecules.

Any of the above methods can be adapted for use in high throughput screening of compound libraries to discover molecules that compete with the EBP for binding to the exosite on BACE. Standard methods can be used to adapt the methods described above for measurements in micro-well plates of varying formats including, but not limited to, 96, 384 and 1536 wells per plate. In a common high throughput screening application, the BACE enzyme and EBP are mixed at fixed concentrations in each well of the micro-well plate. To individual wells of each plate is added one compound of a compound library at a fixed concentration. After mixing the signal associated with BACE/EBP complex formation is measured by use of an appropriate microplate reading instrument. Library compounds that alter the signal associated with BACE/EBP complex formation can thus be identified as potential competitors for the EBP binding site on BACE. These library compounds can then be characterized further to determine their individual binding affinity for BACE by the more complete methods described above.

The present invention provides a method for identifying inhibitors as therapeutics for disorders involved in APP processing and beta-amyloid production comprising:

(a) contacting BACE with a candidate BACE exosite binding compound; and

(b) determining the amount of inhibition of APP processing and beta-amyloid production.

The present invention provides a cell based assay for identifying BACE exosite binding compounds that inhibit beta-amyloid production comprising:

(a) contacting a candidate BACE exosite binding compound with a cell that expresses a beta amyloid precursor protein and BACE wherein the cell is capable of secreting beta-amyloid protein in the absence of the candidate exosite binding compound; and

(b) determining whether the candidate exosite binding compound reduces the amount of beta amyloid protein secreted by the cell.

In another embodiment of the invention, the method for identifying BACE exosite binding compounds that inhibit beta-amyloid production is performed using cell membranes or in a cell-free setting using cell-free enzyme and cell-free substrate according to methods known to those skilled in the art.

The present invention provides EBPs that bind to a BACE exosite and inhibit BACE activity. Inhibition of BACE activity by the EBPs of the present invention can be demonstrated using beta-amyloid precursor protein (also referred to herein as “β-APP” or “APP”), the precursor for Aβ, which through the activity of secretase enzymes is processed into Aβ. Secretase enzymes known in the art have been designated β secretase, which generates the N-terminus of Aβ, α secretase cleaving around the 16/17 peptide bond in Aβ, and γ secretase which generates C-terminal Aβ fragments ending at position 38, 39, 40, 41, 42, and 43, or C-terminal extended precursors which are subsequently truncated to the above peptides.

In accordance with the present invention, full length human APP, known mutations thereof (e.g., the Swedish mutant), fragments of human wild type or mutant APP, peptides derived from human wild type or mutant APP as well as APP or APP fragments fusion proteins, such as, MBP-APP (which includes APP residues 547-595) can be used as a substrate to confirm inhibition of BACE activity by an EBP. The peptide bond hydrolysis activity of BACE can be determined by contacting an appropriate substrate with the enzyme under optimized reaction conditions and then measuring the loss of substrate or production of hydrolysis products as a function of reaction time by some suitable analytical detection method. For example, the recombinant catalytic domain of human BACE can be incubated with the peptide MCA-EVNLDAEFK(-dnp)-COOH (SEQ ID NO:107) in which MCA is a 7-methoxycoumarin-4-acetyl group and dnp is a dinitrophenyl group appended to the epsilon amino group of the lysine side chain. This peptide sequence reflects the amino acid sequence surrounding the beta cleavage site of Swedish mutant APP. The MCA group is highly fluorescent but its fluorescence is quenched by proximity to the dnp group. Thus, the peptide displays low fluorescence signal when intact, but the fluorescence signal is greatly augmented upon BACE-mediated hydrolysis of the peptide.

After a fixed time of incubation, the increase in fluorescence signal can be used as a measure of BACE activity, as described more fully in Mallender et al., (2001) Mol. Pharmacol. 59:619-626 and in Marcinkeviciene et al., (2001) J. Biol. Chem. 276, 23790-23794. The ability of an EBP to inhibit the BACE-mediated hydrolysis of this substrate would be reflected in a diminished fluorescence signal after substrate incubation with BACE in the presence of the EBP.

Such assays can be performed in a cell free setting, using cell-free enzyme and cell-free substrate, or can be performed in a cell-based assay, or using cell membranes according to methods known to those skilled in the art.

The present invention further provides a method of treating a neurological disorder comprising administering to a patient in need of such treatment a therapeutically effective amount of a compound that inhibits beta-amyloid production, or a pharmaceutically acceptable salt or prodrug form thereof, wherein the compound binds to a BACE exosite and effects a decrease in production of beta-amyloid.

The compounds determined from the present invention can be administered orally using any pharmaceutically acceptable dosage form known in the art for such administration. The active ingredient can be supplied in solid dosage forms such as dry powders, granules, tablets or capsules, or in liquid dosage forms, such as syrups or aqueous suspensions. The active ingredient can be administered alone, but is generally administered with a pharmaceutical carrier. A valuable treatise with respect to pharmaceutical dosage forms is Remington's Pharmaceutical Sciences (17^{th} ed.), Mack Publishing Co., Easton, Pa., (1985).

The compounds determined from the present invention can be administered in such oral dosage forms as tablets, capsules (each of which includes sustained release or timed release formulations), pills, powders, granules, elixirs, tinctures, suspensions, syrups, and emulsions. Likewise, they may also be administered in intravenous (bolus or infusion), intraperitoneal, subcutaneous, or intramuscular form, all using dosage forms well known to those of ordinary skill in the pharmaceutical arts. An effective but non-toxic amount of the compound desired can be employed to prevent or treat neurological disorders related to beta-amyloid production or accumulation, such as Alzheimer's disease and Down's Syndrome.

The compounds of this invention can be administered by any means that produces contact of the active agent with the agent's site of action in the body of a host, such as a human or a mammal. They can be administered by any conventional means available for use in conjunction with pharmaceuticals, either as individual therapeutic agents or in a combination of therapeutic agents. They can be administered alone, but generally administered with a pharmaceutical carrier selected on the basis of the chosen route of administration and standard pharmaceutical practice.

The dosage regimen for the compounds determined from the present invention will, of course, vary depending upon known factors, such as the pharmacodynamic characteristics of the particular agent and its mode and route of administration; the species, age, sex, health, medical condition, and weight of the recipient; the nature and extent of the symptoms; the kind of concurrent treatment; the frequency of treatment; the route of administration, the renal and hepatic function of the patient, and the effect desired. An ordinarily skilled physician or veterinarian can readily determine and prescribe the effective amount of the drug required to prevent, counter, or arrest the progress of the condition.

Advantageously, compounds determined from the present invention may be administered in a single daily dose, or the total daily dosage may be administered in divided doses of two, three, or four times daily.

The compounds identified using the present invention can be administered in intranasal form via topical use of suitable intranasal vehicles, or via transdermal routes, using those forms of transdermal skin patches well known to those of ordinary skill in that art. To be administered in the form of a transdermal delivery system, the dosage administration will, of course, be continuous rather than intermittent throughout the dosage regimen.

In the methods of the present invention, the compounds herein described in detail can form the active ingredient, and are typically administered in admixture with suitable pharmaceutical diluents, excipients, or carriers (collectively referred to herein as carrier materials) suitably selected with respect to the intended form of administration, that is, oral tablets, capsules, elixirs, syrups and the like, and consistent with conventional pharmaceutical practices.

For instance, for oral administration in the form of a tablet or capsule, the active drug component can be combined with an oral, non-toxic, pharmaceutically acceptable, inert carrier such as lactose, starch, sucrose, glucose, methyl cellulose, magnesium stearate, dicalcium phosphate, calcium sulfate, mannitol, sorbitol and the like; for oral administration in liquid form, the oral drug components can be combined with any oral, non-toxic, pharmaceutically acceptable inert carrier such as ethanol, glycerol, water, and the like. Moreover, when desired or necessary, suitable binders, lubricants, disintegrating agents, and coloring agents can also be incorporated into the mixture. Suitable binders include starch, gelatin, natural sugars such as glucose or lactose, corn sweeteners, natural and synthetic gums such as acacia, tragacanth, or sodium alginate, carboxymethylcellulose, polyethylene glycol, waxes, and the like. Lubricants used in these dosage forms include sodium oleate, sodium stearate, magnesium stearate, sodium benzoate, sodium acetate, sodium chloride, and the like. Disintegrators include, without limitation, starch, methyl cellulose, agar, bentonite, xanthan gum, and the like.

The compounds determined from the present invention can also be administered in the form of liposome delivery systems, such as small unilamellar vesicles, large unilamallar vesicles, and multilamellar vesicles. Liposomes can be formed from a variety of phospholipids, such as cholesterol, stearylamine, or phosphatidylcholines.

Compounds of the present invention may also be coupled with soluble polymers as targetable drug carriers. Such polymers can include polyvinylpyrrolidone, pyran copolymer, polyhydroxypropylmethacrylamide-phenol, polyhydroxyethylaspartamide-phenol, or polyethyleneoxide-polylysine substituted with palmitoyl residues.

Furthermore, the compounds determined from the present invention may be coupled to a class of biodegradable polymers useful in achieving controlled release of a drug, for example, polylactic acid, polyglycolic acid, copolymers of polylactic and polyglycolic acid, polyepsilon caprolactone, polyhydroxy butyric acid, polyorthoesters, polyacetals, polydihydropyrans, polycyanoacylates, and crosslinked or amphipathic block copolymers of hydrogels.

Gelatin capsules may contain the active ingredient and powdered carriers, such as lactose, starch, cellulose derivatives, magnesium stearate, stearic acid, and the like.

Similar diluents can be used to make compressed tablets. Both tablets and capsules can be manufactured as sustained release products to provide for continuous release of medication over a period of hours. Compressed tablets can be sugar coated or film coated to mask any unpleasant taste and protect the tablet from the atmosphere, or enteric coated for selective disintegration in the gastrointestinal tract. Liquid dosage forms for oral administration can contain coloring and flavoring to increase patient acceptance. In general, water, a suitable oil, saline, aqueous dextrose (glucose), and related sugar solutions and glycols such as propylene glycol or polyethylene glycols are suitable carriers for parenteral solutions. Solutions for parenteral administration preferably contain a water soluble salt of the active ingredient, suitable stabilizing agents, and if necessary, buffer substances. Antioxidizing agents such as sodium bisulfite, sodium sulfite, or ascorbic acid, either alone or combined, are suitable stabilizing agents. Also used are citric acid and its salts and sodium EDTA. In addition, parenteral solutions can contain preservatives, such as benzalkonium chloride, methyl- or propyl-paraben, and chlorobutanol. Suitable pharmaceutical carriers are described in Remington's Pharmaceutical Sciences (1985).

### Crystals of the Present Invention

The crystal forms described herein comprise a BACE polypeptide sequence bound to an active site inhibitor. The crystalline forms comprise an exosite binding site of the BACE protein. In one embodiment, the crystalline form contains a mutated BACE polypeptide. In another embodiment, the crystalline form contains the mature BACE protein.

### Producing a BACE Polypeptide

The BACE polypeptides of the present invention can be produced by methods known in the art, such as they can be synthesized chemically, recombinantly in a cell free system, recombinantly within a cell or can be isolated from a biological source. Chemical synthesis of a BACE polypeptide or a BACE exosite binding site of the present invention can be carried out using a variety of art recognized methods, including stepwise solid phase synthesis, semi-synthesis through the conformationally-assisted re-ligation of peptide fragments, enzymatic ligation of cloned or synthetic peptide segments, and chemical ligation.

### Isolation from a Biological Source

In yet another embodiment, a BACE polypeptide, can be isolated from any suitable tissue source, for example brain tissue. A BACE polypeptide can also be isolated from various species, including but not limited to, mouse and human. Methods for purifying a BACE protein are known and may be used to attain the required level of purity including, for example, reversed-phase high-pressure liquid chromatography (HPLC) using an alkylated silica column such as C₄—, C₈- or C₁₈-silica. A gradient mobile phase of increasing organic content is generally used to achieve purification, for example, acetonitrile in an aqueous buffer, usually containing a small amount of trifluoroacetic acid. Ion-exchange chromatography can be also used to separate peptides based on their charge. Size exclusion can also be used to separate peptides based upon their size. Any of these methods can be employed to obtain a BACE polypeptide as described herein.

In another embodiment, a BACE polypeptide can be isolated from a biological sample using standard protein purification methodology known to those of the art (see, e.g., Janson, Protein Purification: Principles, High Resolution Methods, and Applications, (2^{nd} ed.) Wiley, New York, (1997); Rosenberg, Protein Analysis and Purification: Benchtop Techniques, Birkhauser, Boston, (1996); Walker, The Protein Protocols Handbook, Humana Press, Totowa, N.J., (1996); Doonan, Protein Purification Protocols, Humana Press, Totowa, N.J., (1996); Scopes, Protein Purification: Principles and Practice, Springer-Verlag, New York, (1994); Harris, Protein Purification Methods: A Practical Approach, IRL Press, New York, (1989), all of which are incorporated in their entireties herein by reference).

### Recombinant Methods

Well-established molecular biology, microbiology, recombinant DNA and protein chemistry techniques can be employed to produce a DNA sequence encoding a BACE polypeptide. Such techniques are fully explained in the literature (see, e.g., Sambrook et al, Molecular Cloning: A Laboratory Manual, (3^{rd} ed.) Cold Spring Harbor Laboratory Press, Cold Spring Harbor, N.Y., USA (2001); Glover, DNA Cloning: A Practical Approach, (2^{nd} ed.) IRL Press, New York, USA (1995); Hames & Higgins, Protein Expression: A Practical Approach, Oxford University Press, New York, USA, (1999); Masters, Animal Cell Culture: A Practical Approach, Oxford University Press, New York, USA (2000); Perbal, A Practical Guide To Molecular Cloning (2^{nd} ed.) Wiley, New York, N.Y., USA (1988); Current Protocols in Molecular Biology, (Ausubel et al, eds.), Greene Publishing Associates and Wiley-Interscience, New York (2002); Ausubel, Short Protocols in Molecular Biology: A Compendium of Methods from Current Protocols in Molecular Biology, (4^{th} ed.) John Wiley & Sons, New York, N.Y., USA (1999), all of which are incorporated herein).

Upon providing a nucleic acid sequence encoding a BACE polypeptide (e.g., SEQ ID NOs:112 or 113), or a BACE mutant, analog, derivative or functional equivalent, the encoded polypeptide can be expressed. To express a biologically active BACE polypeptide, a nucleotide sequence encoding a BACE polypeptide, or a BACE mutant, analog, derivative or functional equivalent thereof, can be inserted into an appropriate expression vector and incubated under conditions suitable for expression of the protein.

In one embodiment of the present invention, an expression vector contains a polynucleotide sequence encoding a BACE polypeptide or a sequence as set forth in SEQ ID NOs:110 or 112, encoding a BACE polypeptide or a functional fragment thereof, in which the BACE polypeptide comprises the amino acid sequence as set forth in SEQ ID NOs:111 or 113. In another embodiment, the BACE polypeptide comprises an N-terminal T7 tag.

Expression vectors derived from retroviruses, adenovirus, herpes or vaccinia viruses, or from various bacterial plasmids can be used in the present invention. Methods, which are known to those of ordinary skill in the art, can be used to construct expression vectors containing sequences encoding one or more BACE polypeptides along with appropriate transcriptional and translational control elements. These methods include in vitro recombinant DNA techniques, synthetic techniques, and in vivo genetic recombination. Such techniques are described, for example, in Current Protocols in Molecular Biology, (Ausubel et al., eds.), Greene Publishing Associates and Wiley-Interscience, New York (2002); Ausubel, Short Protocols in Molecular Biology: A Compendium of Methods from Current Protocols in Molecular Biology, (4^{th} ed.) John Wiley & Sons, New York, N.Y., USA (1999); and Sambrook et al., Molecular Cloning: A Laboratory Manual, (3^{rd} ed.) Cold Spring Harbor Laboratory Press, Cold Spring Harbor, N.Y., USA (2001).

The present invention also relates to expression vectors containing genes encoding analogs, derivatives and mutants of a BACE polypeptide, including modified BACE proteins of the present invention, that have the same or homologous functional activity as a BACE polypeptide, and homologs thereof. Such cloning vectors can be prepared as described. Thus, the production and use of derivatives, analogs and mutants related to BACE are within the scope of the present invention.

Recombinant molecules can be introduced into host cells via transfection, electroporation, microinjection, transduction, cell fusion, DEAE dextran, calcium phosphate precipitation, lipofection (lysosome fusion), use of a gene gun, or a DNA vector transporter (see, e.g., Wu et al., (1992) J. Biol. Chem. 267:963-967; Wu & Wu, (1988) J. Biol. Chem. 263:14621-14624).

Any suitable vector-host systems known in the art can be employed in the present invention. Examples of suitable vectors include, but are not limited to, plasmids, such as pBR322 derivatives or pUC plasmid derivatives, e.g., pGEX vectors, pmal-c, pFLAG, etc.

Examples of host-expression vector systems that can be utilized to express a DNA sequence encoding a BACE polypeptide include microorganisms transformed with recombinant bacteriophage DNA, plasmid DNA or cosmid DNA expression vectors containing a DNA sequence encoding a BACE polypeptide; yeast transformed with recombinant yeast expression vectors containing a DNA sequence encoding a BACE polypeptide; insect cell systems infected with recombinant virus expression vectors (e.g., baculovirus) containing a DNA sequence encoding a BACE polypeptide; or animal cell systems. The expression elements of these systems vary in their strength and specificities.

In mammalian host cells, a number of viral-based expression systems can be utilized. In cases where an adenovirus is used as an expression vector, sequences encoding a polypeptide of the present invention can be ligated into an adenovirus transcription/translation complex containing the late promoter and tripartite leader sequence. Insertion into a non-essential E1 or E3 region of the viral genome can be used to obtain a viable virus which is capable of expressing an BACE polypeptide in infected host cells (see, e.g., Logan & Shenk, (1984) Proc. Natl. Acad. Sci. USA 81:3655-3659). In addition, transcription enhancers, such as the Rous sarcoma virus (RSV) enhancer, may be used to increase expression in mammalian host cells. Other expression systems can also be used, such as, but not limited to yeast, plant, and insect vectors.

Yeast-based systems can be employed to express a recombinant polypeptide of the present invention. Techniques for transforming yeast cells with exogenous DNA to produce recombinant polypeptides therefrom are disclosed by, for example, U.S. Pat. Nos. 4,599,311; 4,931,373; 4,870,008; 5,037,743; and 4,845,075, which are incorporated herein by reference. Transformation systems for other yeasts, including Hansenula polymorpha, Schizosaccharomyces pombe, Kluyveromyces lactis, Kluyveromyces fragilis, Ustilago maydis, Pichia pastoris, Pichia guillermondii, and Candida maltosa are known in the art.

Bacterial systems can also be employed to express a recombinant polypeptide of the present invention. In bacterial systems, a number of expression vectors can be selected, depending upon the use intended for the expressed BACE polypeptide product. For example, pET21a (Novagen, Darmstadt, Germany) or pGEX vectors (Promega, Madison, Wis.) can be used to express foreign polypeptides as fusion proteins with glutathione S-transferase (GST). In general, such fusion proteins are soluble and can be easily purified from lysed cells by adsorption to glutathione-agarose beads followed by elution in the presence of free glutathione. Proteins made in such systems can be designed to include, for example, heparin, thrombin, or Factor Xa protease cleavage sites so that the cloned polypeptide of interest can be released from the GST moiety at will.

Host cells transformed with a nucleotide sequence encoding a polypeptide of the present invention can be cultured under conditions suitable for the expression and recovery of the protein from cell culture. The protein produced by a recombinant cell may be secreted or contained intracellularly depending on the sequence and/or the vector used. Some constructs can be used to join nucleic acid sequences encoding a polypeptide to a nucleotide sequence encoding a polypeptide domain, which can facilitate purification of soluble proteins. Such purification facilitating domains include, but are not limited to, metal chelating peptides such as histidine-tryptophan modules that allow purification on immobilized metals; protein A domains that allow purification on immobilized immunoglobulin; and the domain utilized in the FLAG® extension/affinity purification system (available from Immunex Corp., Seattle, Wash.).

The inclusion of cleavable linker sequences such as those specific for Factor Xa or enterokinase (Invitrogen Corp., San Diego, Calif., USA) between the purification domain and the polypeptide can be used to facilitate purification. One such expression vector provides for expression of a fusion protein containing a polypeptide of the present invention with an N-terminal hexa-histidine tag and thrombin cleavage site. The hexa-histidine tag facilitates purification on immobilized metal ion affinity chromatography (IMAC) as described by Porath et al., (1992) Prot. Exp. Purif. 3:263-281, while the thrombin cleavage site provides a means for removing that tag from the fusion protein to purify the BACE.

### Method of Forming BACE Crystals

Crystals can be formed by co-crystallizing a BACE polypeptide, analog, derivative, mutant or functional equivalent with a ligand known or suspected to bind to the BACE polypeptide. Such a co-crystal can be formed by employing the techniques disclosed herein and known to those of ordinary skill in the art. In one aspect, the present invention provides a crystal comprising a BACE polypeptide or fragment and a ligand which is an active site inhibitor.

The formation of BACE crystals can depend on a number of different parameters, including pH, temperature, protein concentration, the nature of the solvent and precipitant, as well as the presence of ligands.

The crystals and fragments thereof disclosed in the present invention can be obtained by a variety of techniques, including batch, liquid bridge, vapor diffusion, and free interface diffusion. Seeding of the crystals can be useful in obtaining X-ray quality crystals. Standard micro and/or macroseeding of crystals can therefore be used in the context of the present invention. In one embodiment, hanging or sitting drop methods are used for the crystallization of BACE polypeptides and fragments thereof.

In an example of a hanging drop method, a drop comprising an amount of BACE polypeptide is mixed with an equal volume of reservoir buffer and grown at about 20° C. until crystals form. General guidance and methods for forming crystals are known in the art (MacPherson, Crystallization of Biological Macromolecules, Cold Spring Harbor Press, Cold Spring Harbor, N.Y., USA (1999), incorporated herein by reference) and can be employed in the context of the present invention to form crystals comprising BACE, and/or fragments thereof.

### Co-Crystals Comprising a BACE Polypeptide and Active Site Inhibitor Soaked with Exosite Peptide

In one aspect of the present invention, a crystal comprising a BACE polypeptide or fragment can also comprise a binding peptide or ligand. In the present invention, for example, the “Soaked” form of BACE is a co-crystal of a BACE polypeptide in complex with an active site inhibitor that has been soaked with an exosite peptide. Crystals of a BACE polypeptide, analog, derivative, mutant or functional equivalent complexed with an active site inhibitor can be soaked with an exosite ligand known or suspected to bind to the BACE polypeptide exosite. Soaking of such a co-crystal can be performed by employing the techniques disclosed herein and known to those of ordinary skill in the art.

### Acquisition and Processing of Diffraction Data

Once a co-crystal of the present invention comprising a BACE polypeptide and active site inhibitor that has been soaked with exosite peptide is available, X-ray diffraction data can be collected. Crystals can be prepared for diffraction using known methodology (see, e.g., Buhrke et al., A Practical Guide for the Preparation of Specimens for X-ray Fluorescence and X-ray Diffraction Analysis, Wiley-VCH, New York, N.Y., USA (1998); and Rodgers (4994) Structure 2, 1135-1140 and/or Garmen & Schneider (1997) J. Appl. Crystallogr. 30, 211-237, both of which are incorporated herein by reference).

A number of ways exist in which meaningful diffraction data can be generated. Examples of area electronic detectors for acquiring diffraction data include charge coupled device detectors, multi-wire area detectors and phosphoimager detectors (Amemiya, (1997) Methods in Enzymology, Vol. 276. Academic Press, San Diego, pp. 233-243; Westbrook & Naday, (1997) Methods in Enzymology, Vol. 276. Academic Press, San Diego, pp. 244-268; 1997. & Kahn & Fourme, Methods in Enzymology, Vol. 276. Academic Press, San Diego, pp. 268-286).

In one embodiment, a suitable system for diffraction data collection might include a Bruker AXS Proteum R system, equipped with a copper rotating anode source, Confocal Max-Flux® optics and a SMART 6006 charge coupled device detector. Collection of x-ray diffraction patterns are well documented by those skilled in the art (See, for example, Ducruix and Geige, 1992, IRL Press, Oxford, England). In another embodiment, a suitable system for diffraction collection might include a Rigaku FR-E copper rotating anode source with Rigaku Confocal MicroMax® optics and a Rigaku Saturn92 charge coupled device detector. In another embodiment, a suitable system for diffraction collection might include a Rigaku FR-E copper rotating anode source with Rigaku Confocal Max-Flux HR® optics and a Rigaku R-axis IV++ image plate detector. In a further embodiment, a suitable system for diffraction collection might include an Advanced Photon Source beamline ID17 with a Area Detector System Corporation Q210 mosaic (2×2) charge coupled device detector.

### Determining a Three-dimensional Structure of the Present Invention

After acquiring X-ray diffraction data from a crystal comprising a BACE polypeptide, the three-dimensional structure of the polypeptide can be determined by analyzing the diffraction data. Such an analysis can be employed whether the polypeptide is a wildtype polypeptide or a fragment thereof, or a mutant, derivative or analog of an BACE polypeptide.

X-ray diffraction data can be used to determine a structure by employing available software packages such as HKL2000 with its component programs DENZO and SCALEPACK; (Otwinowski, Z & Minor, W., (1997) p. 307-326. in Carter and Sweet (ed.), Methods Enzymol., Macromolecular Crystallography part A, vol. 276. Academic Press, Inc., New York, N.Y.; D*TREK (Rigaku), MOSFLM (Leslie, A. G. W. (1992) Joint CCP4+ESF-EAMCB Newsletter on Protein Crystallography, No. 26), XDS (Kabsch, W. (1993) J. Appl. Crystallogr. 26: 795-800.) to integrate the data.

A number of ways exist in which meaningful diffraction data can be phased. Data can be phased by MR, MIR, SIR, MIRAS, SIRAS, and MAD techniques using software such as the CCP4 package (SERC Collaborative Computing Project No. 4, Daresbury Laboratory, UK, 1979); SHARP (GlobalPhasing, Ltd), PHASER, refinement programs such as CNX (Brünger, A. T., P. D. Adams, G. M. Clore, W. L. DeLano, P. Gros, R. W. Grosse-Kunstleve, J.-S. Jiang, J. Kuszewski, M. Nilges, N. S. Pannu, R. J. Read, L. M. Rice, T. Simonson, and G. L. Warren. 1998. Crystallography & NMR system: a new software suite for macromolecular structure determination. Acta Crystallogr. Sect D 54:905-921.), BUSTER/TNT (GlobalPhasing Ltd), REFMAC (CCP4). Molecular graphics programs are utilized that are capable of displaying electron density and manipulating atomic coordinates such as QUANTA (Accelrys, 2005 & preceding), COOT (Emsley, P. & Cowtan, K (2004) Acta Crystallogr. Sect D 60:2126-2132), O (Jones et al., (1991) Acta Cryst. A 47, 110-119); CHAIN (J. Sack (1988) J. Mol. Graphics. 6: 224-225), MIFIT (Rigaku: http://www.moleculariamges.com/MIFit.html).

In one approach, a molecular replacement (MR) technique is utilized. The molecular replacement method refers to a method that involves generating a preliminary atomic model of a crystal, whose structural coordinates are unknown, by orienting and positioning a related crystal structure whose structural coordinates are known. Phases are calculated from this model and combined with the observed amplitudes to give an approximate Fourier synthesis of the structure whose coordinates are unknown. From this preliminary atomic model, electron density map analysis can be performed to identify the location of exosite peptides that are bound to BACE. High quality electron density maps allow for the building of atomic coordinates of the exosite peptide into the appropriate unassigned electron density. This, in turn, can be subject to any of the several forms of refinement, as defined herein, to provide a final, accurate structure of the unknown crystal. Lattman, E., “Use of the Rotation and Translation Functions,” in Methods in Enzymology, 115, pp. 55 77 (1985); M. G. Rossmann, ed., “The Molecular Replacement Method”, Int. Sci. Rev. Ser., No. 13, Gordon & Breach, New York, (1972).

Using the structural coordinates of a BACE polypeptide model along with processed diffraction data from a BACE/active site inhibitor co-crystal soaked with exosite peptide, molecular replacement may be used to determine the structural coordinates of crystalline BACE/active site inhibitor and exosite binding peptide, or exosite modulator compound.

The present invention therefore provides a method for determining the three-dimensional structure of a crystallized BACE polypeptide in complex with an active site inhibitor and exosite peptide to a resolution of about 6.0 Å or better.

### Representative Applications of the Present Invention

The present invention, which comprises, in part, the structural coordinates of Tables 7-8, has broad-based utility and can be employed in many applications. Representative applications include modulator design, mutant design and screening operations. These and other applications are described herein.

### Modulator Screening, Design and Identification

The BACE exosite binding site structural coordinates provided herein facilitate structure-based or rational drug design and virtual screening to design or identify potential ligands and/or modulators of a BACE polypeptide. The structural features of the exosite binding site of a BACE polypeptide, as described by the structural coordinates herein, provide insights into the BACE exosite binding site that, prior to the present invention, were unknown. By providing these features, structure-based BACE modulator design and virtual screening efforts can now be performed.

In a representative modulator design approach, a three dimensional model of an BACE polypeptide can be used to identify structural and chemical features that might be involved in binding of ligands to an exosite binding site of a BACE polypeptide. Identified structural or chemical features can then be employed to design ligands or modulators of a BACE polypeptide or identify test molecules as ligands or modulators of a BACE polypeptide.

Those of ordinary skill in the art can employ one of several methods to virtually screen chemical entities or fragments for their ability to associate with a BACE exosite binding site, or a structurally similar polypeptide. This process can begin by visual inspection of, for example, the exosite binding site on the computer screen based on the structural coordinates provided herein in Tables 7-8 or the structural coordinates of a model generated using the structural coordinates of Tables 7-8. After inspection of the target protein (e.g., BACE) a particular chemical entity can be examined by visual inspection or by computer modeling using a docking program such as DOCK, AutoDock, GOLD, or FlexX (Kitchen et al., (2004) Nature Reviews Drug Discovery 3:935-949) or Glide™ (Schrödinger, LLC, New York, N.Y., 2005 and prior) to determine its potential as a modulator. This procedure can include computer fitting of chemical entities to a target protein in order to determine if the structure of the chemical entity will complement or interfere with the structure of the subject polypeptide (Bugg et al., Scientific American December 1993:92-98; West et al., (1995) TIPS 16:67-74). A compound that has been designed or selected to function as a modulator should spatially fit into a binding site when it is associated with a BACE polypeptide. A docking operation can be followed by energy minimization and molecular dynamics with standard molecular mechanics forcefields, such as CHARMM, AMBER, MMFF, and OPLS2001.

As described herein, a modulator can be identified in a screen or it can be designed de novo or using a library of chemical fragments as a starting point. A variety of computational methods for molecular design are known. See, for example, Cohen et al., (1990) J. Med. Chem. 33: 883-894; Kuntz et al., (1982) J. Mol. Biol. 161: 269-288; DesJarlais, (1988) J. Med. Chem. 31: 722-729; Bartlett et al., (1989) Spec. Publ., Roy. Soc. Chem. 78: 182-196; Goodford et al., (1985) J. Med. Chem 28: 849-857; and DesJarlais et al., J. Med. Chem. 29: 2149-2153. As an alternative to designing a modulator de novo, a library or a database of molecules can be screened in silico to identify candidate modulators. Examples of databases that can be screened include ACD (Molecular Designs Limited), NCI (National Cancer Institute), CCDC (Cambridge Crystallographic Data Center), CAST (Chemical Abstract Service) Derwent (Derwent Information Limited), Maybridge (Maybridge Chemical Company Ltd), Aldrich (Aldrich Chemical Company), DOCK (University of California in San Francisco), and the Directory of Natural Products (Chapman & Hall).

A docking operation can be performed as part of a modulator design process or it can be performed to learn more about how a given ligand associates or might associate with a given structure. Thus, in one aspect, the present invention also provides a method of docking a ligand, modulator or candidate modulator with a structure. In one embodiment, the method comprises positioning a candidate modulator into a BACE exosite binding site, or a part of BACE exosite binding site, wherein the binding site is a described by the structural coordinates Tables 7-8. The method can further comprise analyzing structural and chemical feature complementarity of the candidate modulator With all or a part of a BACE polypeptide.

Accordingly, the present invention provides a method of designing a modulator of a BACE polypeptide comprising: (a) modeling all or a part of a BACE exosite binding site; and (b) based on the modeling, designing a candidate modulator that has structural and chemical features that are complementary to all or a part of the BACE exosite binding site; wherein the BACE exosite binding site is defined by the structural coordinates of Tables 7-8. After a candidate modulator has been identified, the candidate modulator can then be synthesized and tested for modulation ability in a suitable assay.

The method can further comprise: (c) docking the designed candidate modulator into all or a part of the BACE polypeptide; and (d) analyzing the structural and/or chemical feature complementarity of the candiate modulator with all or a part of the BACE polypeptide, specifically a BACE exosite binding site. The method can also comprise analyzing structural and chemical feature complementarity of a second chemical entity with all or a part of an BACE polypeptide.

In still another embodiment, the present invention provides a method for identifying or designing a modulator to a polypeptide of the invention comprising the steps of (a) providing a computer modeling application with a set of structural coordinates of a molecule or complex, the molecule or complex including at least a portion of an BACE polypeptide; (b) providing the computer modeling application with a set of structural coordinates for a chemical entity; (c) evaluating the potential binding interactions between the chemical entity and active site of the molecule or molecular complex; (d) structurally modifying the chemical entity to yield a set of structural coordinates for a modified chemical entity, and (e) determining whether the modified chemical entity is expected to bind to the molecule or complex, wherein binding to the molecule or complex is indicative of potential modulation of the polypeptide of the invention.

In still another embodiment, the present invention provides a method of identifying a modulator of BACE comprising (a) providing the atomic coordinates of a BACE exosite binding site provided in one of Tables 7-8 defining a three-dimensional structure of the BACE exosite binding site; (b) using the three-dimensional structure to design or select a test compound by computer modeling; (c) synthesizing or acquiring the test compound; and (d) determining the ability of the test compound to modulate a biological activity of a BACE polypeptide, wherein a difference in the biological activity of the BACE polypeptide observed in the presence and absence of the test compound indicates the test compound is a modulator of the BACE polypeptide.

In yet another aspect of the present invention, a method of designing a modulator of a BACE polypeptide is disclosed and comprises: (a) designing a potential modulator of a BACE polypeptide that will interact with amino acids in an exosite binding site of the BACE polypeptide, based upon a three-dimensional structure comprising a BACE exosite binding site alone or in complex with a ligand; (b) synthesizing the modulator; and (c) determining whether the potential modulator modulates the activity of the BACE polypeptide.

The structural coordinates of the present invention can also be employed in the refinement of an existing BACE polypeptide modulator. By refining the structure of an existing modulator, desirable properties of the modulator can be enhanced. Thus, in another aspect of the present invention a method of increasing the efficiency of a modulator of an BACE polypeptide is disclosed and comprises: (a) providing a first ligand having a known effect on the biological activity of an BACE polypeptide; (b) modifying the first ligand based on an evaluation of a three-dimensional structure of an BACE polypeptide to form a modified ligand; (c) synthesizing the modified ligand; and (d) determining an effect of the modified ligand on a BACE polypeptide, wherein the efficiency of a modulator of a BACE polypeptide is increased if the modified ligand favorably alters a biological activity of a BACE polypeptide with respect to the biological activity of the first ligand. This method can be employed in an iterative fashion and repeated until a desired level of improvement is attained.

### In Silico Screening Operations

The BACE exosite binding site structural coordinates of the present invention can be employed in structure-based modulator design. Virtual screening methods, e.g., methods of evaluating the potential of chemical entities to bind to the exosite binding site, are known in the art. These methods often employ databases as sources of candidate modulators and often are employed in designing modulators. Often these methods begin by visual inspection of a binding site of a target polypeptide on the computer screen. Selected candidate modulators can then be placed, e.g., docked, in one or more positions and orientations within the binding site and chemical and structural feature complementarity can be analyzed.

In virtual screening, molecular docking is sometimes followed by energy minimization and molecular dynamics with standard molecular mechanics forcefields such as CHARMM and MMFF represents a typical approach. Examples of computer programs which can assist in the selection of chemical entities useful in the present invention include, but are not limited to, GRID (Goodford, (1985) J. Med. Chem. 28:849-857; Boobbyer et al., (1989) J. Med. Chem. 32:1083-1094), AUTODOCK (Goodsell et al., (1990) Proteins: Structure, Function, and Genetics 8:195-202), DOCK (Kuntz, (1982) J. Mol. Biol. 161:269-288), and Glide™ (Schrödinger, LLC, New York, N.Y., 2005 and prior). Databases of chemical entities that may be used include, but are not limited to, ACD (Molecular Designs Limited, San Leandro, Calif.), Aldrich (Aldrich Chemical Company), NCI (National Cancer Institute), Maybridge (Maybridge Chemical Company Ltd), CCDC (Cambridge Crystallographic Data Center, Cambridge, UK), CAST (Chemical Abstract Service) and Derwent (Derwent Information Limited).

A virtual screening approach can include, but is not limited to, the steps of: (a) selecting a candidate modulator from a database and positioning one or more molecular conformations of the candidate modulator in one or more orientations within all or a part of a binding site of a target molecule, the conserved backbone residues of the binding site having a root mean square deviation of not more than about 6.0 Å from the structural coordinates of the BACE amino acids that are within 6.0 Å of any exosite peptide atom E316, K317, F318, P319, F322, G325, E326, Q327, L328, V329, C330, W331, Q332, A333, T335, D372, V373, A374, S376, D378, D379, C380, Y381 (SEQ ID NO:113) according to Tables 7-8; (b) characterizing structural and chemical features of the candidate modulator and exosite binding site; (c) selecting a second candidate modulator adapted to join with or replace the docked candidate modulator and fit spatially into all or a part of an BACE exosite binding site; (d) evaluating the docked candidate modulator using one or more scoring schemes which account for structural and chemical feature complementarity.

Upon selection of one or more preferred chemical entities, their relationship to each other and to a BACE polypeptide can be visualized. Multiple chemical entities can be assembled into a single candidate modulator. Programs useful in assembling the individual chemical entities include, but are not limited to, SYBYL (Tripos, St. Louis Mo., USA), LEAPFROG (Tripos, St. Louis Mo., USA), LUDI (Bohm, (1992) J. Comp. Aid. Mol. Design. 6:61-78, Accelrys, San Diego, Calif.) and 3D Database systems (see, e.g., Martin, (1992) J. Med. Chem. 35(12):2145-2154), as discussed herein.

Accordingly, the present invention provides an in silico method for evaluating the ability of a chemical moiety to bind to a BACE exosite binding site or a structurally similar molecule comprising: (a) docking a candidate modulator into a BACE exosite binding site on a BACE polypeptide, as described by the structural coordinates of Tables 7-8; and (b) analyzing structural and chemical feature complementarity between the candidate modulator and all or a part of the BACE polypeptide. One site that can be useful as a site into which a ligand can be docked is an BACE polypeptide exosite binding site comprising amino acids that are within 6 Å of any exosite peptide atom E316, K317, F318, P319, F322, G325, E326, Q327, L328, V329, C330, W331, Q332, A333, T335, D372, V373, A374, S376, D378, D379, C380, Y381 (SEQ ID NO:113).

The method can further comprise a step in which a second candidate modulator is joined to the first candidate modulator that was docked and analyzed, and the resultant candidate modulator is docked and analyzed. Candidate modulators designed or identified using the methods described herein can subsequently be synthesized and screened in a BACE activity or binding assay. The method can also comprise evaluating the potential of a chemical entity to associate with a BACE exosite binding site, and candidate modulators can be screened using computational means and biological assays to identify ligands and modulators of a BACE polypeptide.

In another embodiment, a method of performing an in silico screen comprises the steps of: (a) docking a candidate modulator into all or a part of a BACE exosite binding site, wherein the BACE exosite binding site is described by the structural coordinates of Tables 7-8; (b) analyzing structural and chemical feature complementarity between the candidate modulator and all or a part of the BACE exosite binding site; (c) synthesizing the candidate modulator; and (d) screening the candidate modulator in a biological assay for the ability to modulate a BACE polypeptide. The method can further comprise one or more of the following steps: (e) screening the candidate modulator in an assay that characterizes binding to a BACE exosite binding site; In this and all methods described herein, a modulator of a BACE polypeptide can induce one or more of the following activities of BACE presented in this non-inclusive list: inhibition of APP processing and beta-amyloid production.

The term “all or a part of a BACE polypeptide” relates to enough of a BACE polypeptide so as to be useful in docking or modeling a ligand into the exosite binding site, although it is not necessary to employ a complete BACE polypeptide. Preferably, a BACE exosite binding site comprises the following residues that are within 6.0 Å of any exosite peptide atom E316, K317, F318, P319, F322, G325, E326, Q327, L328, V329, C330, W331, Q332, A333, T335, D372, V373, A374, S376, D378, D379, C380, Y381 (SEQ ID NO:113).

### Designing a Mutant BACE Polypeptide

As used herein, the term “mutation” includes one or more amino acid deletions, insertions, inversions, repeats, or substitutions as compared to a native protein (e.g., an BACE polypeptide). Various methods of making mutations are known to one of ordinary skill in the art. A mutant can have the same, similar, or altered biological activity as compared to the native protein. The structural coordinates of the present invention can be employed in the design of a mutant BACE polypeptide or fragment thereof. The structural coordinates describe, in one aspect, various structural features of a BACE exosite binding site. Those of ordinary skill in the art can employ this understanding of the BACE structure to select one or more amino acid residues for mutation. The rationale for selecting a residue can be based on steric, chemical or other considerations. Thus, the present invention provides for the generation of BACE exosite mutants, and the ability to determine the crystal structures of those that crystallize. Further, desirable sites for mutation can be identified, based on analysis of the three-dimensional BACE structural coordinates provided herein.

In one aspect, the present invention provides a method of designing a mutant comprising making one or more amino acid mutations in a BACE polypeptide. The mutant so designed can comprise a complete BACE polypeptide or a portion of thereof, such as a BACE exosite binding site. In some embodiments, a mutant comprises an addition, a deletion or a substitution of one or more of the amino acids of an BACE exosite binding site. One embodiment of a method of designing a mutation comprises: (a) selecting a property of a BACE polypeptide to be investigated; (b) providing a three-dimensional structure of a BACE polypeptide; and (c) evaluating the structure to identify a residue known or suspected to related to the selected property. The steps of the method can be repeated a desired number of times.

Initially a feature of a BACE polypeptide to be investigated is selected, for example the ability to bind a ligand. Other properties can also be investigated and a combination of properties can be investigated with a single mutation.

Next, a three-dimensional structure of a BACE polypeptide is provided. The three-dimensional structure can be described by all or a part of the structural coordinates of Tables 7-8. The structure is then evaluated to identify a residue known or suspected to relate to the selected property. The evaluating can be of any form and can be dependent on the nature of the property being investigated. The evaluating can start with the substitution, addition or deletion of one or more residues for one or more BACE polypeptide residues. After the alteration(s) is performed, a visual inspection of the three-dimensional structure as it is displayed on a computer screen can be performed. Alternatively, the evaluating can comprise one or more calculations to determine the effect of a given alteration. Further, calculations can be performed that can quantitatively assess the effect of a given mutation on the charge, hydrophobicity, etc., either locally or globally.

When it is desired to introduce a mutation into a BACE polypeptide amino acid sequence (e.g., a mutation designed using structural coordinates of the present invention, such as by a method disclosed herein) this can be accomplished by any method known to those of skill in the art, including site-directed mutagenesis of DNA encoding an BACE polypeptide. Examples of suitable mutagenesis techniques, include oligonucleotide mediated mutagenesis, alanine scanning, PCR mutagenesis, site directed mutagenesis, cassette mutagenesis, and restriction selection mutagenesis.

A mutant BACE of the present invention will have the same or similar biological activity as the native BACE polypeptide or a portion thereof and can be used for any purpose for which the native is used. Thus, the present invention provides an BACE polypeptide, or a mutant portion thereof, comprising one or more amino acid mutations, addition or deletion in a wildtype BACE polypeptide. A mutant portion of a BACE polypeptide can comprise a mutant exosite binding site.

Examples of BACE mutants include but are not limited to allelic genes, homologous genes from other species, which are altered by the substitution of different codons that encode the same amino acid residue within the sequence, thus producing a silent change. Likewise, a modified BACE polypeptide derivative of the present invention can include, but is not limited to, derivatives containing, as a primary amino acid sequence, all or part of the amino acid sequence of a BACE polypeptide, including altered sequences in which functionally equivalent amino acid residues are substituted for residues within the sequence resulting in a conservative amino acid substitution. For example, one or more amino acid residues within a BACE sequence can be substituted with another amino acid of a similar polarity, hydrophobicity, charge, etc. which acts as a functional equivalent. It is generally preferable for initial substitutions to be conservative, e.g., the replacement group will have approximately the same size, shape, hydrophobicity and charge as the original group. Further, amino acids and structural elements known in the art to alter conformation should generally be avoided, unless such an alteration is desired.

Included within the scope of the term “mutant” are chimeric and fusion proteins. Such chimeras or fusion proteins can include, for example, a secretion signal or an additional heterologous functional region. For instance, a region of additional amino acids, particularly charged amino acids, can be added to the N-terminus of the polypeptide to improve stability and persistence in the host cell, during purification, or during subsequent handling and storage. Also, peptide moieties can be added to the polypeptide to facilitate purification. Such regions may be removed prior to final preparation of the polypeptide. The addition of peptide moieties to polypeptides to engender secretion or excretion, to improve stability and to facilitate purification, among others, are familiar and routine techniques in the art. One common example of a fusion protein comprises a heterologous region from immunoglobulin that is useful to solubilize proteins.

Mutagenesis methods as disclosed herein can be combined with high-throughput, automated screening methods to detect activity of cloned, mutagenized polypeptides in host cells. Mutagenized DNA molecules that encode active polypeptides (e.g., cell proliferation) can be recovered from the host cells and rapidly sequenced using modern equipment. These methods allow the rapid determination of the importance of individual amino acid residues in a polypeptide of interest, and can be applied to polypeptides of unknown structure.

### Method of Identifying a Modulator of BACE

The BACE structural coordinates provided herein facilitate structure-based BACE modulator design and identification. Thus, according to another aspect of the present invention, there is provided a method of identifying a test compound which modulates at least one activity of an BACE polypeptide and which comprises contacting a BACE polypeptide with the test compound to determine whether modulation of the BACE polypeptide occurs. The test compound is preferably selected or designed using the defined three-dimensional structure of the BACE exosite binding site as described herein. Modulators of BACE activity have value, e.g., as tools for studying the mechanisms of BACE activity, and for regulating APP processing and beta-amyloid production. More importantly, these modulators provide lead compounds for drug development for the treatment of a variety of conditions including but not limited to neurological diseases, such as Alzheimer's disease. Modulators of the invention have therapeutic utility (1) in treating diseases caused by inhibition of BACE in tissues where it is customarily found and (2) in treating diseases whose symptoms can be ameliorated by inhibiting BACE activity.

Therefore, in one aspect, the invention provides a method of identifying a modulator of BACE which comprises (a) providing the atomic coordinates of a BACE exosite binding site provided in one of Tables 7-8 defining a three-dimensional structure of a BACE exosite binding site; (b) using the three-dimensional structure to design or select a test compound by computer modeling; (c) synthesizing or acquiring the test compound; and (d) contacting the test compound with BACE to determine the ability of the test compound to modulate a biological activity of BACE. Differences in biological activity observed in the presence and absence of the test compound indicates the test compound is a modulator of BACE.

In one embodiment, the step of using the three-dimensional structure to design or select a test compound by computer modeling comprises (a) identifying chemical entities or fragments with the potential to bind a BACE exosite binding site; and (b) assembling the identified chemical entities or fragments into a single molecule to provide the structure of the test compound. The assembly of fragments can be accomplished with molecule building/editing facilities within molecular modeling packages such as SYBYL (Tripos, Inc., St. Louis Mo., USA) or Maestro™ (Schrödinger, LLC, New York, N.Y., 2005 and prior), with de novo compound design packages such as LEAPFROG (Tripos, St. Louis Mo., USA), LUDI (Bohm, (1992) J. Comp. Aid. Mol. Design. 6:61-78, Accelrys, San Diego, Calif.), or similar computer programs.

The methods of the present invention may utilize any means of monitoring or detecting the desired BACE activity. For example, in one embodiment, the test compound is capable of providing a detectable signal in response to inhibition of BACE by measuring the difference in the detectable signal in the presence and in the absence of the test compound and thereby identifying the test compound as a modulator of BACE. In another embodiment, the method identifies a test compound that decreases said detectable signal and is a BACE inhibitor. In another embodiment, the method identifies a test compound that increases the detectable signal and is an BACE activator. In a further embodiment, the test compound is labelled with at least one fluorescent donor dye and the signal is detected by a Fluorescence Resonance Energy Transfer (FRET) assay, for example.

In another embodiment, the relative amounts of BACE between a cell population that has been exposed to the test compound to be tested compared to an unexposed control cell population may be assayed. In this format, probes such as specific antibodies are used to monitor the differential expression of BACE in the different cell populations. Cell lines or populations are exposed to the test compound to be tested under appropriate conditions and time. Cellular lysates or membrane fractions may be prepared from the exposed cell line or population and a control, unexposed cell line or population. The cellular lysates or membrane fractions are then analyzed with the probe.

Typically animal cells, including mammalian cells are useful in these assays for testing modulators of BACE as the cells must have intracellular mechanisms which permit the receptor to be displayed on the cell surface. Of particular use are Xenopus laevis frog oocytes, which typically utilize cRNA rather than standard recombinant expression systems proceeding from the DNA encoding the desired protein. Capped RNA (at the 5′ end) is typically produced from linearized vectors containing DNA sequences encoding the receptor. The reaction is conducted using RNA polymerase and standard reagents. cRNA is recovered, typically using phenol/chloroform precipitation with ethanol and injected into the oocytes.

The animal host cells expressing the DNA encoding BACE or the cRNA-injected oocytes are then cultured to effect the expression of the encoding nucleic acids so as to produce the receptor display on the cell surface. These cells then are used directly in assays for assessment of the potential modulator to bind, inhibit, or activate the receptor.

Another method of evaluating modulators as potential therapeutic agents typically involves a binding assay in which the test compound (such as a peptide or a small organic molecule) would be tested to measure if, or to what extent, it binds a BACE exosite binding site. Preferably, a mammalian or insect cell line that expresses BACE or plasma membrane preparations thereof, will be used in a binding assay. For example, a candidate antagonist competes for binding to a BACE polypeptide with either a labeled peptide agonist or antagonist. Varying concentrations of the test compound are supplied, along with a constant concentration of the labeled substrate, agonist or antagonist. The inhibition of binding of the labeled material by the test compound can then be measured using established techniques. This measurement is then correlated to determine the amount and potency of the modulator that is bound to BACE.

Another method of evaluating test compounds for potential therapeutic applications typically involves a functional assay in which the test compound's effect upon cells expressing the recombinant receptor is measured (e.g., its ability to affect BACE's ability to process APP), rather than simply determining its ability to bind the receptor (see Jantzen et al. (1999) Thromb. Haemost. 81:111-117). Suitable functional assays include measuring the APP processing and/or beta-amyloid production in the presence of varying concentrations of test compounds.

In another aspect, the invention provides methods for identifying a test compound useful for modulating BACE activity in vivo, comprising contacting the test compound with an animal and determining the effect, if any, on BACE activity. An effect on BACE activity, or any associated phenomenon can be determined in comparison to a suitable control. Suitable controls include animals of the same species that have not been contacted with the test compound.

A wide variety of assays may be used for this purpose, including in vivo behavioral studies, physiological analyses, and the like. Depending on the particular assay, whole animals may be used or cells derived therefrom. Cells may be freshly isolated from an animal, or may be immortalized in culture.

The modulators of the present invention can be, for example, peptides or small molecules. A skilled artisan can readily recognize that there is no limit as to the structural nature of the modulators of BACE. Dominant negative proteins, DNAs encoding these proteins, antibodies to these proteins, peptide fragments of these proteins or mimics of these proteins may also be introduced into cells to affect function. “Mimic” used herein refers to the modification of a region or several regions of a peptide molecule to provide a structure chemically different from the parent peptide but topographically and functionally similar to the parent peptide (see Grant G A. in: Meyers (ed.) Molecular Biology and Biotechnology (New York, VCH Publishers, 1995), pp. 659-664).

The modulators of BACE can be prepared using standard solid phase (or solution phase) peptide synthesis methods, as is known in the art. In addition, the DNA encoding these peptides may be synthesized using commercially available oligonucleotide synthesis instrumentation and produced recombinantly using standard recombinant production systems. The production using solid phase peptide synthesis is necessitated if non-gene-encoded amino acids are to be included.

The modulators of the present invention can be provided alone, or in combination with other agents that modulate a particular pathological process. For example, a modulator of BACE can be administered in combination with other known drugs. As used herein, two modulators are said to be administered in combination when they are administered simultaneously or are administered independently in a fashion such that the agents will act at the same time.

The modulators of BACE can be administered via parenteral, subcutaneous, intravenous, intramuscular, intraperitoneal, transdermal, or buccal routes. Alternatively, or concurrently, administration may be by the oral route. The dosage administered will be dependent upon the age, health, and weight of the recipient, kind of concurrent treatment, if any, frequency of treatment, and the nature of the effect desired.

The present invention further provides compositions containing one or more agents which modulate expression or at least one activity of BACE. While individual needs vary, determination of optimal ranges of effective amounts of each component is within the skill of the art. Typical dosages comprise 0.1 to 100 mg/kg body wt. The preferred dosages comprise 0.1 to 10 mg/kg body weight. The most preferred dosages comprise 0.1 to 1 μg/kg body weight.

In addition to the pharmacologically active agent, the modulators of the present invention may contain suitable pharmaceutically acceptable carriers comprising excipients and auxiliaries which facilitate processing of the active compounds into preparations which can be used pharmaceutically for delivery to the site of action. Suitable formulations for parenteral administration include aqueous solutions of the active compounds in water-soluble form, for example, water-soluble salts. In addition, suspensions of the active compounds as appropriate oily injection suspensions may be administered. Suitable lipophilic solvents or vehicles include fatty oils, for example, sesame oil, or synthetic fatty acid esters, for example, ethyl oleate or triglycerides. Aqueous injection suspensions may contain substances which increase the viscosity of the suspension include, for example, sodium carboxymethyl cellulose, sorbitol, and/or dextran. Optionally, the suspension may also contain stabilizers. Liposomes can also be used to encapsulate the agent for delivery into the cell.

The pharmaceutical formulation for systemic administration according to the invention may be formulated for enteral, parenteral or topical administration. Indeed, all three types of formulations may be used simultaneously to achieve systemic administration of the active ingredient.

Suitable formulations for oral administration include hard or soft gelatin capsules, pills, tablets, including coated tablets, elixirs, suspensions, syrups or inhalations and controlled release forms thereof.

In another aspect, the invention provides a method of identifying a compound that modulates BACE comprising (a) obtaining a crystal of a complex comprising a BACE exosite binding site and a molecule; (b) obtaining the atomic coordinates of the crystal; (c) using the atomic coordinates and one or more molecular techniques to identify a compound that modulates BACE activity; (d) assaying the inhibitory properties of the compound by administering it to a cell or cell extract of BACE; and (e) detecting BACE activity, wherein an increase or decrease in BACE activity indicates that the compound is a modulator of BACE.

As used herein, a cellular extract refers to a preparation or fraction which is made from a lysed or disrupted cell, for instance, from fibroblasts. The preferred source of cellular extracts will be cells that normally express the receptor polypeptide.

A variety of methods can be used to obtain an extract of a cell. Cells can be disrupted using either physical or chemical disruption methods. Examples of physical disruption methods include, but are not limited to, sonication and mechanical shearing. Examples of chemical lysis methods include, but are not limited to, detergent lysis and enzyme lysis. A skilled artisan can readily adapt methods for preparing cellular extracts in order to obtain extracts for use in the present methods.

Once an extract of a cell is prepared, the extract is mixed with a modulator of BACE under conditions in which association of the modulator with BACE can occur. A variety of conditions can be used, the most preferred being conditions that closely resemble conditions found in the cytoplasm of a human cell. Features such as osmolarity, pH, temperature, and the concentration of cellular extract used, can be varied to optimize the association of the protein with the binding partner.

After mixing under appropriate conditions, the bound complex is separated from the mixture. A variety of techniques can be utilized to separate the mixture. For example, antibodies specific to BACE can be used to immunoprecipitate the BACE:modulator complex. Alternatively, standard chemical separation techniques such as chromatography and density/sediment centrifugation can be used.

After removal of non-associated cellular constituents found in the extract, the modulator can be dissociated from the complex using conventional methods. For example, dissociation can be accomplished by altering the salt concentration or pH of the mixture.

To aid in separating associated BACE:modulator complexes from the mixed extract, BACE can be immobilized on a solid support. For example, the BACE protein can be attached to a nitrocellulose matrix or acrylic beads. Attachment of the BACE protein to a solid support aids in separating peptide/binding partner pairs from other constituents found in the extract. Modulators may be identified using a Far-Western assay according to the procedures of Takayama et al. Methods in Molecular Biology, Vol. 69 (1997) pp. 171-184 or identified through the use of epitope tagged proteins or GST fusion proteins.

Alternative assays may be performed to measure a difference in BACE activity by the test compound as compared to a control. Other assays can include measuring the level of beta-amyloid in the presence of varying concentrations of test compounds and/or measuring the activity of biological pathways that include or are modulated by BACE substrates, for example APP processing, in the presence of varying concentrations of test compounds.

### EXAMPLES

The following examples as set forth herein are meant to illustrate and exemplify the various aspects of carrying out the present invention and are not intended to limit the invention in any way.

The synthetic peptides described herein were prepared as N-terminal acetyl derivatives and as C-terminal carboxy amides, with the exception of those peptides identified as SEQ ID NOs:19 and 48, which were prepared as N-terminal acetyl derivatives but did not contain a C-terminal carboxy amide group.

### Example 1

### BACE Exosite Binding Peptides from Solid Phase Panning at pH 7.0

Two highly selected and homologous 12 mer phage peptides bound BACE specifically and reproducibly in phage-ELISA tests. Bristol-Myers Squibb fUSE5-based C4C, C6C, 5- and 15 mer libraries, and M13-based C7C libraries, and 7- and 12 mer libraries obtained from New England Biolabs, Beverly, Mass. were panned for three cycles against BACE (produced as described herein in Example 3). BACE was immobilized by coating at 0.5 μg/well in 4 wells of Dynex Immulon 4HBX plates overnight at 4° C. in 0.1M NaHCO₃ buffer, pH 9.0. Panning was by standard procedures at room temperature that involved blocking wells with 2% BSA in PBS and elution with 0.1M HCL, pH 2.2. The vector NTI alignment tool and visual inspection of sequences were employed to analyze the selected peptides.

After sequencing approximately 20-50 clones from each library after three cycles of selection, we prepared essentially all possible candidate clones (39 clones in total) for phage-ELISA to obtain direct evidence for affinity to BACE. Eleven clones gave binding signals and one of those clones, a 12 mer clone (NLTTYPYFIPLP (SEQ ID NO:19)), was reproducibly shown to specifically bind to BACE. We therefore sequenced additional 12 mer clones to try to find additional candidate clones. Eleven candidate clones were tested by phage ELISA and one clone (ALYPYFLPISAK (SEQ ID NO:20)) exhibited specific binding to BACE. This ALYPYFLPISAK (SEQ ID NO:20) peptide is homologous to NLTTYPYFIPLP (SEQ ID NO:19) and consistent with the specific binding of both those peptides to BACE. The ALYPYFLPISAK (SEQ ID NO:20) peptide and NLTTYPYFIPLP (SEQ ID NO:19) peptides were the two most efficiently recovered clones with 13 and 9 copies, respectively.

### Example 2

### BACE Binding Peptides from Solid- and Solution Phase Panning at pH 5.0

Solid phase panning experiments at pH 7.0 yielded exosite BACE binding peptides BMS-561871 and BMS-561877 which share a conserved core region. Solution and solid phase panning at pH 5.0 yielded 21 peptides with essentially the same conserved core region that is present in BMS-561871 and BMS-561877. Overall, solution phase panning appeared to facilitate the isolation of these peptides. This is consistent with the idea that the peptide binding site on BACE may be less accessible when BACE is immobilized, as in solid phase panning. The presence of the active site inhibitor OM99-2 in solid phase panning did not noticeably improve the ability to recover these peptides. In the absence of OM99-2, any selection for peptides that occupy the active site of BACE may therefore be less efficient or absent. The results are consistent with the idea that the new set of peptides binds BACE outside the active site.

Phage ELISA indicated that all 21 peptides from panning at pH 5.0 bind BACE specifically at pH 5.0 and pH 7.0. Binding specificity for peptides from solution phase was only tested at pH 5.0 and where signals were obtained (subject to phage concentrations which were not standardized), peptides bound specifically. Consensus peptides are provided at the end of Table 1. The presence of a histidine residue immediately flanking the YPYF (SEQ ID NO:1) motif, i.e. HYPYF (SEQ ID NO:8), appears to contribute to more efficient binding at pH 5.0. BMS-561871 and BMS-561877 were isolated at pH 7.0 and lack histidine at this position.

### Peptides with Other Sequence Motifs

Two tight-binding peptides contained a conserved region that is significantly different from, but clearly related to the core region in BMS-561871 and BMS-561877. Solid- and solution phase panning yielded 6 and 4 groups of peptides, respectively, that contained motifs other than the YPYF motif in the peptides listed in Table 1. Two peptides from solution panning, ETWPRFIPYHALTQQTLKHQQHT (SEQ ID NO:22) and TAEYESRTARTAPPAPTQHWPFFIRST (SEQ ID NO:23), exhibited strong and specific binding that was similar to phage carrying BMS-561871. The ETWPRFIPYHALTQQTLKHQQHT (SEQ ID NO:22) and TAEYESRTARTAPPAPTQHWPFFIRST (SEQ ID NO:23) BMS-561871 peptides include a WPXFI (SEQ ID NO:21) motif. The result is consistent with the fact that the two peptides ETWPRFIPYHALTQQTLKHQQHT (SEQ ID NO:22) and TAEYESRTARTAPPAPTQHWPFFIRST (SEQ ID NO:23) from solution panning share a region with homology that is different from, but clearly similar to, the core region of the peptides ALYPYFLPISAK (SEQ ID NO:20) and NLTTYPYFIPLP (SEQ ID NO:19). Thus, peptides containing the YPYF (SEQ ID NO:1) motif or closely related sequences are able to efficiently bind BACE at the BACE exosite.

### Panning

a.) Solid Phase:

The M13-based C7C-, 12-, and 15 mer libraries were panned in the presence or absence of 1 μM OM99-2 against BACE produced as BACE-IgG, from CHO cells and treated to remove the Ig-domain, as described in Example 3. Three panning cycles were carried out: BACE was immobilized at 0.5 μg/well in 4 wells of Dynex Immulon plates overnight in 0.1M NaHCO₃ buffer, pH 9.0. This was followed by blocking the wells with PBS+2% BSA for 1 hour. For panning at pH 5.2, blocking buffer was discarded and library phage was then added for two hours in 50 mM NaOAc, pH 5.2+2% BSA, followed by washes with 50 mM NaOAc, pH 5.2, +0.2% Tween 20 and subsequent elution with 0.1M HCl, pH 2.2 for amplification or DNA sequencing after round three. For panning at pH 7.0, all buffers were based on PBS instead of NaOAc.

b.) Solution Phase:

The following mixtures of our M13-based libraries were panned against BACE-prepared from CHO cells (vide supra): a.) C7C+C8C libraries, b.) 12-+15 mer libraries, and c.) 23-+27-+33 mer libraries. Library mixtures and BACE were pre-blocked in 50 mM NaOAc, pH 5.2, +2% BSA and then mixed for two hours. The mixtures were then added to Pansorbin Protein A cells (Calbiochem) in 50 mM NaOAc, pH 5.2, after blocking the cells in NaOAc, pH 5.2, plus 2% BSA and 1% milk. This step was followed by washing the Pansorbin cell-phage complexes several times with 50 mM NaOAc, pH 5.2, plus 0.2% Tween 20. Phage were eluted with 6M urea, pH 3.0, and used for amplification and further panning cycles or DNA sequencing after round three.

### Phage ELISA

Standard procedures were used: BACE (without IgG₁ domain) was coated in 0.1M NaHCO₃, pH 9.0 overnight at 4° C. From this point onwards, all incubation and wash buffers were based on 50 mM NaOAc, pH 5.2, for determining binding at pH 5.2. To determine binding at pH 7.0, NaOAc was replaced by PBS.

TABLE 1

Peptides Having a YPYF Motif Specifically Bind
to a BACE Exosite

SOLID PHASE PANNING AT pH 7.0:

| | |
|---|---|
| NLTTYPYFIPLP (BMS-561871) | (SEQ ID NO:19) |
| ALYPYFLPISAK (BMS-561877) | (SEQ ID NO:20) |

SOLID PHASE PANNING AT pH 5.0:

| | |
|---|---|
| QNHYPYFIAVPI | (SEQ ID NO:24) |
| EGNKHYPYFIKV | (SEQ ID NO:25) |
| THSHYPYFIELE | (SEQ ID NO:26) |
| QQYPYFIPVIRP | (SEQ ID NO:27) |

SOLUTION PANNING AT pH 5.0:

| | |
|---|---|
| HYPYFLPLHTPK | (SEQ ID NO:28) |
| AMLDGAPTNRNSQHYPYFLPIATV | (SEQ ID NO:29) |
| LPVYDTTAPTHYPYFLPLPRISP | (SEQ ID NO:30) |
| EGNKHYPYFIKV | (SEQ ID NO:25) |
| SQLQHYPYFRPL | (SEQ ID NO:31) |
| YIPHYPYFIRLN | (SEQ ID NO:32) |
| KMHSMINQLGTRHYPYFREINDY | (SEQ ID NO:33) |
| GSTKSYPYFIHT | (SEQ ID NO:34) |
| DIWNGAKAPKNSMYPYFIPSSLK | (SEQ ID NO:35) |
| ISVINQPAQNMHPRQMTAYPYFRPISR | (SEQ ID NO:36) |
| DVYPYFVSSNEGHSIRHKGNNSL | (SEQ ID NO:37) |
| YPYFIDSHPPKELMPHSWVQSKYPASPQTHTTY | (SEQ ID NO:38) |
| GYPYFLNLKNSH | (SEQ ID NO:39) |
| NSYPYFIHLSNP | (SEQ ID NO:40) |
| HDYPYFMMLTGH | (SEQ ID NO:41) |
| QIETYPYFLPIL | (SEQ ID NO:42) |
| YYPYFISTAREV | (SEQ ID NO:43) |

Consensus:

| | |
|---|---|
| HYPYFIPL | (SEQ ID NO:18) |
| Y L I | |
| T V V | |
| S M | |

TABLE 2
Peptides with Other Sequence Motifs

SOLUTION PHASE PANNING AT pH 5.0:

| | |
|---|---|
| ETWPRFIPYHALTQQTLKHQQHT | (SEQ ID NO:22) |
| TAEYESRTARTAPPAPTQHWPFFIRST | (SEQ ID NO:23) |

### Example 3

### Isothermal Titration Calorimetry (ITC) of BACE and Exosite Peptides

Isothermal titration calorimetry was performed to determine quantitatively the binding affinities of BMS-561877 and BMS-561871 for β-secretase. Recombinant human BACE was expressed as a fusion protein with human IgG₁, in Chinese hamster ovary (CHO) cells (Vassar et al., (1999) Science 286:735-741 and Haniu et al., (2000) J. Biol. Chem. 275:21099-21106).

This construct, referred to as BACE-T-IgG, also contained a protease cleavage site between BACE and IgG₁, sensitive to human α-thrombin. The cDNA for the catalytic domain of human BACE (residues 1-460) was PCR-amplified and subcloned into the mammalian expression vector pTV1.6, upstream of a thrombin cleavage site linked to cDNA encoding human IgG₁ heavy chain. The vector construct, pTV1.6-BACE-T-IgG, was used to produce stably transfected DHFR-deficient CHO DG44 cells, which were then scaled up using methotrexate for selection.

The clarified growth media harvested from CHO DG44 cells which contained the fusion protein was loaded onto a rProtein A SEPHAROSE™ column (5×20 cm, Amersham Pharmacia Biotech) using a peristaltic pump at 4° C., at 4 mL/min. The column was washed with Dulbecco's PBS, pH 7.1, 4 mL/min, until baseline absorbance at 280 nm was observed. BACE-T-IgG was eluted from the resin with 0.10 M citrate, pH 3.0, into tubes containing 0.5 volumes of 4 M Tris, pH 8. Fractions containing the fusion protein were dialyzed extensively using 12,000-14,000 kDa MWCO membrane (UltraPURE, GIBCO BRL) against PBS, pH 7.1, at 4 ° C. The protein was sterile filtered (0.22 μm) and stored at 4° C.

To generate BACE for binding experiments, the fusion protein BACE-T-IgG was treated with human α-thrombin (Enzyme Research Labs, South Bend, Ind.) at a ratio of 1:500 (mass:mass) in Dulbecco's PBS, pH 7.1, at 37° C. for 2 hr. Human α-thrombin was removed by passing the sample over Benzamidine SEPHAROSE™ 6B column (Amersham Pharmacia Biotech). The cleaved IgG₁ was captured by passing the solution over a rProtein A SEPHAROSE™ (Amersham Pharmacia Biotech) column, whereas the BACE passed through this column. The protein sample was further purified by concentrating to 10-15 mg/mL using a centrifugation concentration unit (Millipore Ultrafree 10 kDa MWCO, 15 mL unit) and loading onto a SUPERDEX™ 200 PC 3.2/30 gel-filtration column (Amersham Pharmacia Biotech). The column was run at 3 mL/min with Dulbecco's PBS, pH 7.1, at room temperature. Fractions containing BACE were combined, sterile filtered (0.22 μm) and stored at 4° C. The protein was characterized by SDS-PAGE and other biophysical techniques, including UV-vis spectrometry, dynamic and static light scattering, to demonstrate that it was glycosylated and monomeric. Amino-terminal sequencing indicated a mixture of two start sequences, LPRET- and ETDEE-, the latter of which is the expected sequence for the mature sequence of the protease (Haniu et al., (2000) J. Biol. Chem. 275:21099-21106).

BACE was prepared for isothermal titrating calorimetry by extensive dialysis against fresh buffer at 4° C. using 12,000-14,000 kDa MWCO dialysis membrane (UltraPURE, GIBCO BRL). The buffer was either Dulbecco's PBS (2 mM KH₂PO₄, 8 mM Na₂HPO₄, 137 mM NaCl, 3 mM KCl), pH 7.1, or 25 mM NaOAc, containing 137 mM NaCl and 3 mM KCl, pH 5.3. Following 2 buffer changes, the protein was removed from the membrane and centrifuged (5 min×4000 g, 4° C.) to remove particulates. The protein was stored at 4° C. until needed for the calorimetry experiments. The protein concentration was determined by 10-fold dilution into the same buffer, and measuring the UV absorbance at 280 nm in a 1.00 cm pathlength cell (calculated value=1.22 AU=1.00 mg/mL protein, based on mean glycosylated molecular weight=53.5 kDa, and given amino acid composition). Final concentrations used in isothermal titrating calorimetry were typically 5.0 μM, containing a final concentration of 0.5% v/v DMSO.

Peptides were dissolved in the buffer dialysate from protein dialysis, and equal volumes of DMSO were added to each (0.5% v/v DMSO). The pH values of the solutions were adjusted as necessary to equal that of the buffer dialysate and protein sample within 0.01 pH units. The concentrations of peptides NLTTYPYFIPLP (SEQ ID NO: 19) and ALYPYPLPISAK (SEQ ID NO:20) were determined by diluting 10-fold in the same buffer and measuring the UV absorbance at 276 nm, using the value of ε=2780 M⁻¹ cm⁻¹ (or 2×1390 M⁻¹ cm⁻¹ for 2 Tyr residues per peptide).

The active site inhibitor peptide OM99-2 was obtained from Bachem (King of Prussia, Pa.) as a dry white powder. The compound was weighed into a clean polypropylene tube (1.7 mL) and DMSO was added to prepare a stock solution of 10.0 mM. This stock sample was diluted in buffer (protein dialysate) to ˜50 μM containing a final concentration of 0.5% v/v DMSO for titration experiments.

Isothermal titrating calorimetry experiments were performed with a VP-ITC instrument from MicroCal, Inc. (Northampton, Mass.). The instrument was controlled with a personal computer, and thermally regulated at the desired experimental temperature (25° C. or 37° C.). Samples of BACE and peptides were degassed for 2×5 min at 15° C. using a temperature-regulated degassing unit (MicroCal) before loading into the sample chamber or syringe, respectively. Deionized, degassed water was loaded into the instrument reference chamber and used for all experiments. For each titration experiment, a fresh sample of BACE (typically 5.0 μM, 2.0 mL) was loaded into the instrument sample chamber (volume=1.438 mL), using a glass syringe, following the manufacturer's directions. Similarly, fresh peptide samples (typically ˜150 μM for peptides NLTTYPYFIPLP (SEQ ID NO:19) and ALYPYPLPISAK (SEQ ID NO:20) and ˜50 μM for OM99-2, 0.3 mL total volume) were loaded into the instrument injecting syringe unit before each experiment. For experiments to demonstrate that the active site directed inhibitor peptide OM99-2 and the peptide NLTTYPYFIPLP (SEQ ID NO:19) did not compete for the same site, a 10-fold excess of desired peptide was first added to a fresh sample of BACE and incubated at room temperature for 5 min before degassing and loading into the instrument. Titrations were then performed with the other peptide in the syringe.

### Instrument Parameters

The temperature was maintained at 25° C. or 37° C. during the titration experiments. A power setting of 6.0 μCal/sec was used, and a syringe stirring rate of 300 rpm was used. The initial injection was kept at 1.5-2.0 μL and the data from this injection was not included in the analysis as a standard practice. To completely define the binding isotherm, typically a 2.5-fold to 3.5-fold excess of peptide was added during the course of the titration experiment, using about 15 injections per molar equivalent, or 3.0 μL (NLTTYPYFIPLP, SEQ ID NO:19) or 6.0 μL (OM99-2) per injection. The data collection time per injection was fixed at 360 sec, with a signal averaging time of 2 sec. The data was analyzed using the manufacturer's software fitting to a single site binding model (i.e. Origin 5.0 for ITC). Before molar heat calculations were done, background corrections were made on all peaks by subtracting the mean of the final 10-15 injections from all injections.

The calculated molar heat values were fitted to a single binding site model using the manufacturer's software to determine the binding stoichiometry (n), the association constant (K_{A}), the enthalpy of the reaction (ΔH), and the entropy of the reaction (ΔS). These values were used to calculate the dissociation constant (K_{d}) which is the reciprocal of K_{A}, and the Gibbs free energy of the reaction (ΔG), which is related to the K_{A}, ΔH, and ΔS by the following equations: ΔG=−RT (ln (K_{A}))=66H−TΔS (Levine, Physical Chemistry, (2^{nd} ed.), McGraw-Hill Co., (1983), p. 125).

The sample cell was cleaned between injections by washing extensively with PBS, H₂O, and again with PBS. After multiple experiments (typically 6-8), the sample cell and syringe were more extensively cleaned using manufacturer's recommendations with a detergent solution heated to 50° C., followed by extensive washing with H₂O, methanol, H₂O, and finally PBS. Blank injections of buffer into buffer were then performed to establish sufficient cleaning and reproducible background before carrying out additional BACE-peptide experiments.

### Results

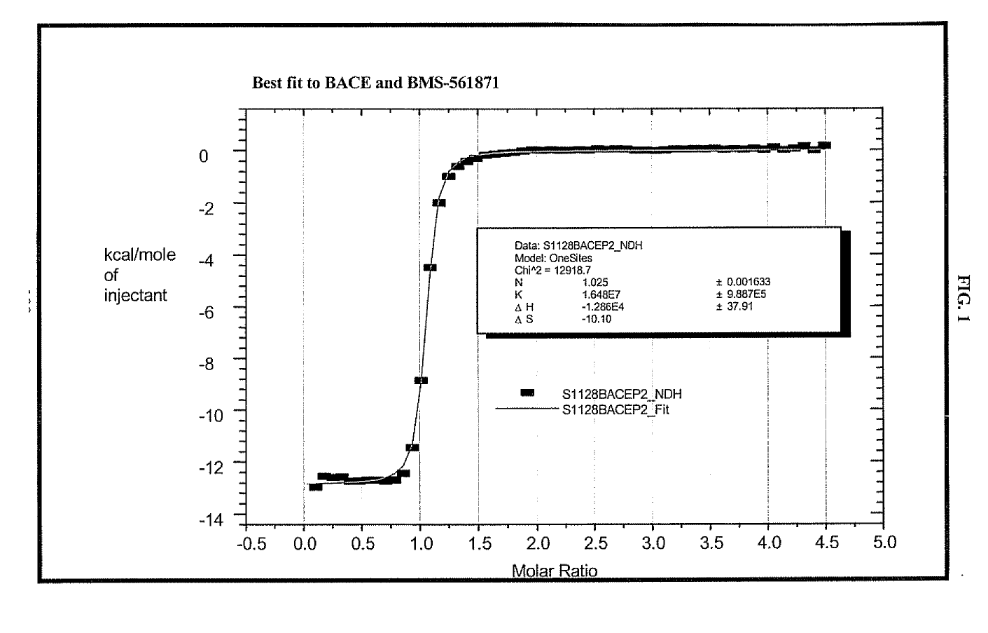

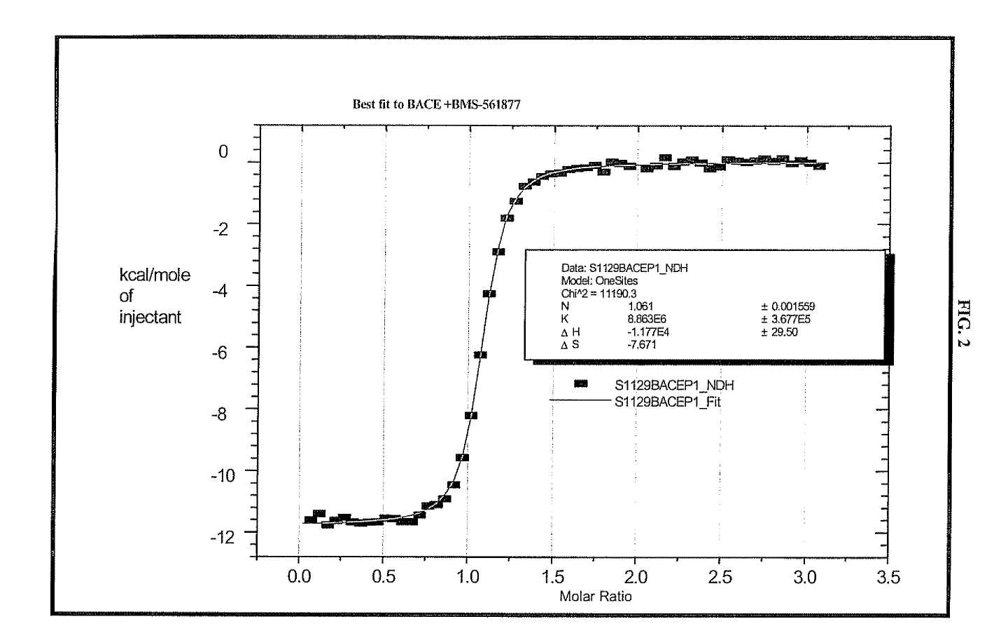

Titrations with peptides NLTTYPYFIPLP (SEQ ID NO:19) and ALYPYPLPISAK (SEQ ID NO:20) into BACE demonstrated saturable 1:1 binding in Dulbecco's PBS, pH 7.1 at 25° C. (below, FIGS. 1-2). The binding constants were determined to be K_{d}=61 nM for NLTTYPYFIPLP (SEQ ID NO:19) and K_{d}=113 nM for ALYPYPLPISAK (SEQ ID NO:20).

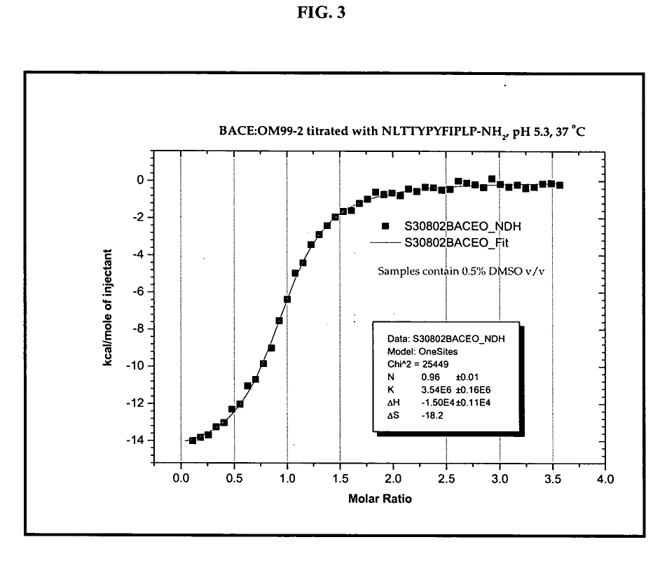

Further experiments were carried out with BACE at pH 5.3 and 37° C. with NLTTYPYFIPLP (SEQ ID NQ: 19) to investigate the binding of this peptide under catalytically active conditions in both the absence and presence of the active site inhibitor peptide OM99-2. Representative integrated data fitted to a single site model are given below in FIG. 3 for the experiment in which a 10-fold excess of OM99-2 was first added to BACE, and the complex was then titrated with NLTTYPYFIPLP (SEQ ID NO:19), at pH 5.3, 37° C. The determined thermodynamic parameters for the complete sets of experiments are given in Table 3, and all experiments showed saturable binding and excellent fits to a single site model.

TABLE 3
Calculated and Fitted Data for Four Experiments with BACE, OM99-2, and Peptide
#1, Conducted at pH 5.3, 37° C.

| | Titrant | ΔG | T(dS) | ΔH | K_{d} | Stoichiometry |
|---|---|---|---|---|---|---|
| Sample Cell Contents | (syringe) | (kcal/mol) | (kcal/mol) | (kcal/mol) | (nM) | (mole:mole) |
| CHO BACE | Peptide #1* | −9.1 | −6.8 | −15.9 | 380 | 0.93:1.00 |
| CHO BACE: OM99-2 | Peptide #1 | −9.3 | −5.7 | −15.0 | 280 | 0.96:1.00 |
| CHO BACE | OM99-2 | −11.9 | −5.3 | −17.2 | 4 | 1.01:1.00 |
| CHOBACE: Peptide #1 | OM99-2 | −12.1 | −4.0 | −16.1 | 3 | 1.03:1.00 |

*Peptide #1 is NLTTYPYFIPLP (SEQ ID NO: 19)

These experiments demonstrated that binding of peptide NLTTYPYFIPLP (SEQ ID NO:19), and OM99-2 to BACE were not mutually exclusive, and that the binding was not strongly coupled, as shown below in Scheme 1.

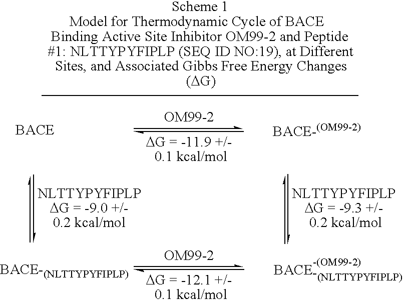

### Example 4

### Fluorescently Labeled EBPs Binding to BACE Assay

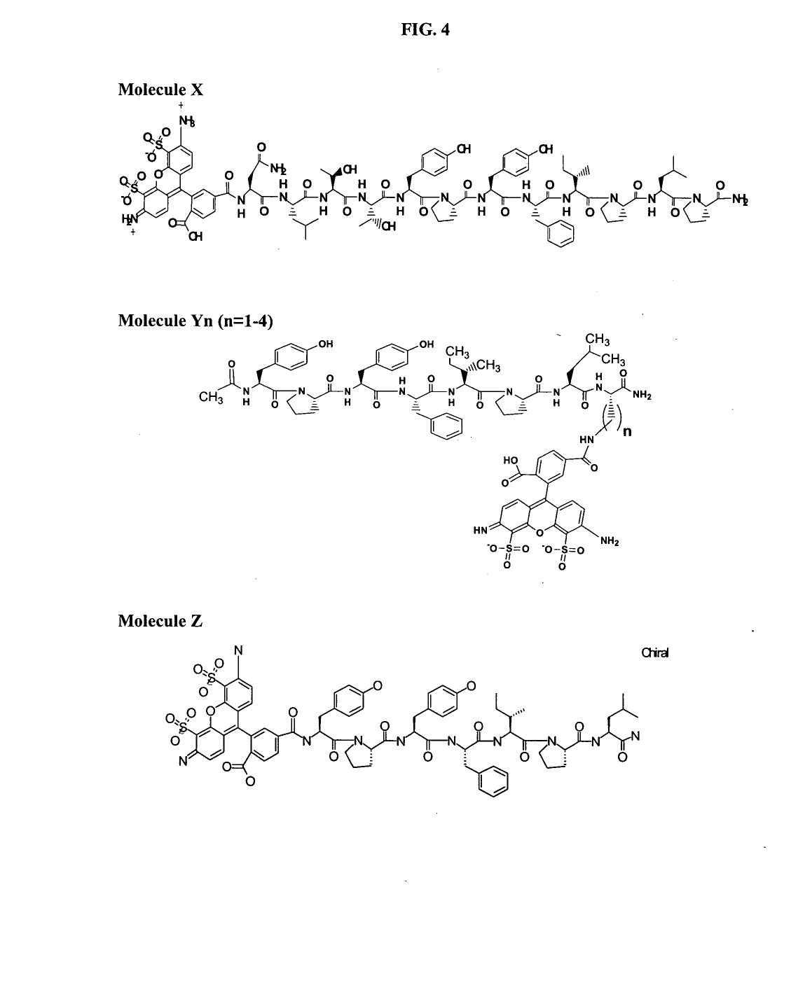

An assay to evaluate the binding of BACE to EBPs labeled with a fluorescent molecule (for example, but not limited to Alexa488) was developed. This assay uses the catalytic domain of human BACE expressed in a CHO cell line (according to Example 3) and labeled EBPs such as Molecule X shown in FIG. 4. The change in fluorescent anisotropy of the EBP peptide upon binding to BACE is monitored.

Peptides were dissolved in 100% DMSO (dimethyl sulfoxide) at 10 mM concentration, and then diluted 10-fold into deionized water. The concentration of the labeled peptides was determined by their absorbance at 495 nm (ε=71000 cm⁻¹ M⁻¹). The concentration of selected unlabeled peptides was determined by their Tyr absorbance at 276 nm (ε=1390 cm⁻¹M⁻¹ per Tyr residue).

The binding was carried out at pH 7.1 (PBS buffer) and pH 4.5 (50 mM acetate buffer) in the presence of 1% DMSO. Fluorescence anisotropy was measured at 25° C. in an AVIV fluorometer. The excitation and emission wavelengths were set to 495 and 519 nm, respectively. The excitation and emission slit width were 4 and 10 nm, respectively. A concentrated BACE stock was used to titrate a 300 μl solution of 10 nM labeled EBP. The final BACE concentration ranges from 10 to 5000 nM. After the addition of BACE, the solution was mixed for 10 times with a pipettor. The fluorescence anisotropy was averaged over a 5 minute period.

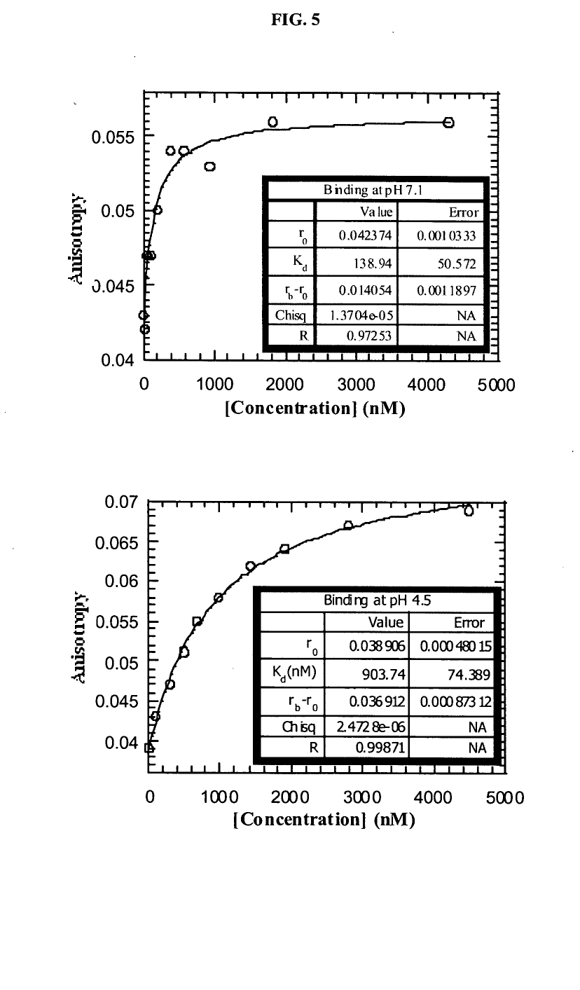

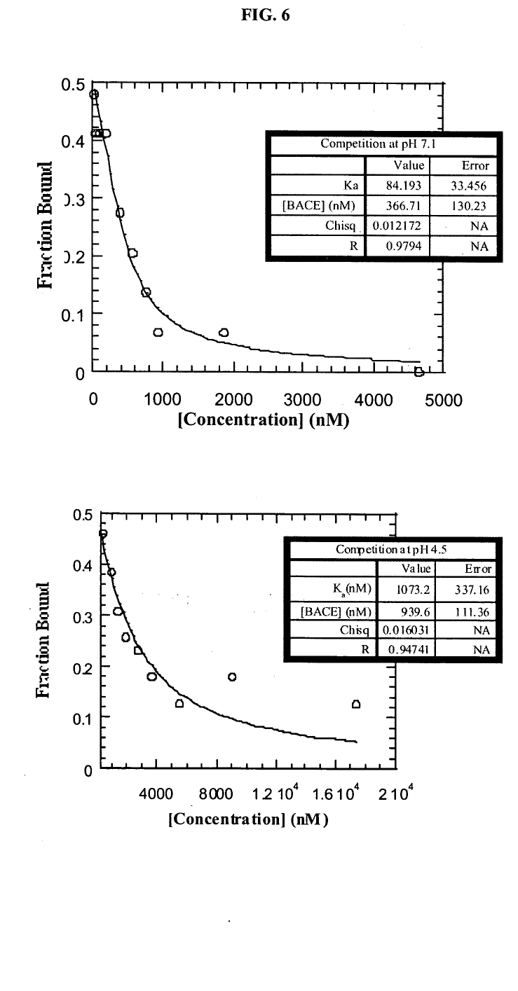

The change in anisotropy was plotted against the BACE concentration (see, for example, FIG. 5). The K_{d} of the labeled EBP as well as the initial (r₀) and final anisotropy (r_{b}) of the labeled EBP were calculated from curve fitting using the program Kaleidagraph. The equation used to fit the binding data is identical to equation 1 in Lai et al., (2000) Arch. Biochem. Biophys. 381:278-284.

### Example 5

### Competitive Binding Assay

The competitive binding assay was carried out at pH 7.1 (PBS buffer) or pH 4.5 (50 mM acetate buffer). The fluorescence anisotropy was measured at 25° C. in an AVIV fluorometer. The excitation and emission wavelengths were set to 495 and 519 nm, respectively. The excitation and emission slit widths were 4 and 10 nm, respectively.

Labeled EBP (Molecule X) at 10 nM was mixed with BACE at a concentration equal to the K_{d}. The initial anisotropy value was measured. A concentrated unlabeled peptide or compound stock was titrated into the above solution of EBP and BACE. The final concentration of unlabeled peptide or compound ranges from 20 to 20000 nM. After each addition, the solution was mixed for 10 times with a pipettor. The fluorescence anisotropy was averaged over a 5 minute period.

The change in anisotropy was converted to fractional occupancy based on the r₀ and r_{b} obtained from the binding assay. The fractional occupancy was then plotted against the concentration of the competing peptide or compound. (see, for example, FIG. 6) The K_{d} of the competing ligand was calculated from curve fitting using the program KALEIDAGRAPH™ (Synergy Software, Reading, Pa.). The equation used to fit the competition data is identical to equation 4 in Lai et al., (2000) Arch. Biochem. Biophys. 381:278-284.

### Example 6

### Binding of Labeled EBP (Molecule X) to BACE at pH 7.1 and pH 4.5

Binding of a labeled EBP (Molecule X, FIG. 4) to BACE was carried out at pH 7.1 and pH 4.5. By fitting of the data to a 1:1 binding model, the binding constants were determined to be 139 and 904 nM at pH 7.1 and pH 4.5, respectively.

### Example 7

### Determination of Binding Affinity of Unlabeled EBP Using the Competition Assay

The binding affinity of peptide NLTTYPYFIPLP (SEQ ID NO:19) (unlabeled Molecule X) was determined using the competition assay as described in Example 6. The binding constants were 84 and 1073 nM at pH 7.1 and pH 4.5, respectively. The ability of unlabeled EBP to displace Molecule X from binding to BACE suggests that the labeled and unlabeled peptides bind to BACE at the same exosite. The binding constants of this EBP with and without Alexa488 label at essentially the same at both pHs, indicating that the presence of the Alexa488 label does not affect the binding interactions of the EBP to BACE. The binding constant at pH 7.1, determined by the fluorescent anisotropy, is consistent with the ITC result (61 nM) of Example 3. At pHs where BACE will be catalytically active, the binding of the EBP NLTTYPYFIPLP (SEQ ID NO:19) is weaker (1073 nM at pH 4.5 by fluorescence anisotropy and 380 nM at pH 5.3 by ITC). Unless otherwise specified, subsequent binding and competition experiments were carried out at pH 4.5, the pH optimum of the proteolytic activity of BACE.

### Example 8

### Screening of Truncated Peptides to Define the Minimal Length Requirement for Binding

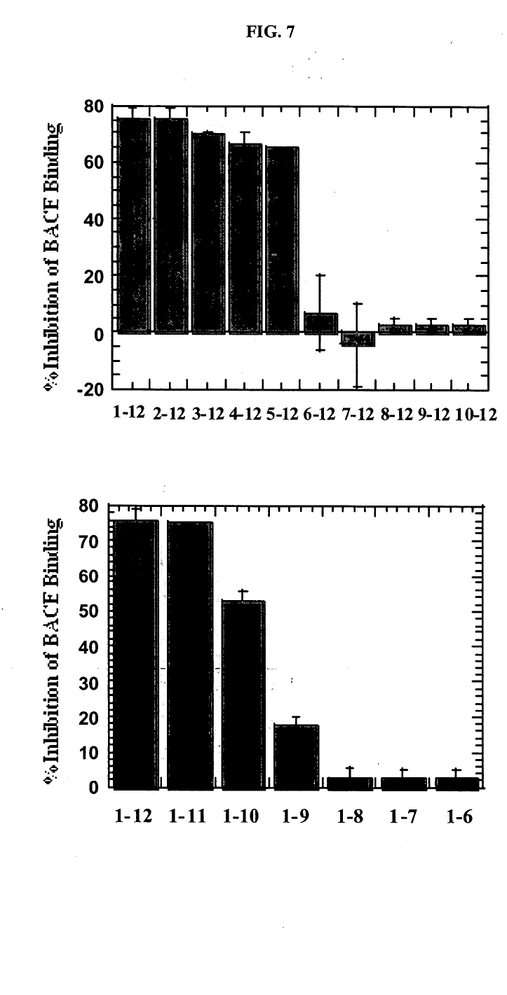

A collection of truncated peptides (from N—, from C—, and from both N- and C-termini of peptide NLTTYPYFIPLP (SEQ ID NO:19)) was screened in the competition assay described above in Example 6 with slight modification. Molecule X was used as the labeled EBP (K_{d}=1.0 μM at pH 4.5). The truncated unlabeled peptides were screened at a single concentration of 10 μM at pH 4.5. The anisotropy values detected with Molecule X in the absence of inhibitor, i.e., truncated unlabeled peptide (rₐ) and in the presence of the inhibitor (r_{b}) and the labeled peptide alone (r₀) were used to calculate the percent of inhibition: 100 (rₐ-r_{b})/(rₐ-r₀). The percent of inhibition was compared among the truncated peptides. It was determined that the N-terminal 4 residues and the C-terminal residue in peptide NLTTYPYFIPLP (SEQ ID NO:19) were not critical in binding (FIG. 7, upper and lower panels). Therefore, it was determined that the minimal length desired for binding is a 7-mer peptide. A preferred 7-mer BACE exosite binding peptide was identified having the sequence of YPYFIPL (SEQ ID NO:10), corresponding to amino acids 5-11 in the original NLTTYPYFIPLP (SEQ ID NO:19) peptide.

In addition to Molecules X, Yn, and Z, the following BACE exosite binding peptides were identified:

| | | |
|---|---|---|
| Compound Number | Sequence | Amino Acid Composition |

| | | |
|---|---|---|
| BMS-561871 | 1-12 | NLTTYPYFIPLP |
| | | (SEQ ID NO:19) |
| BMS-593925 | 5-11 | YPYFIPL |
| | | (SEQ ID NO:10) |
| BMS-590022 | 2-12 | LTTYPYFIPLP |
| | | (SEQ ID NO:44) |
| BMS-590023 | 3-12 | TTYPYFIPLP |
| | | (SEQ ID NO:45) |
| BMS-590024 | 4-12 | TYPYFIPLP |
| | | (SEQ ID NO:46) |
| BMS-590008 | 5-12 | YPYFIPLP |
| | | (SEQ ID NO:47) |
| BMS-590014 | 1-11 | NLTTYPYFIPL |
| | | (SEQ ID NO:48) |
| BMS-599191 | 5-11^{a} | YPYFIAL |
| | | (SEQ ID NO:49) |
| BMS-599192 | 5-11^{a} | YPYFIPA |
| | | (SEQ ID NO:50) |
| BMS-599195 | 5-11^{b} | YPBFIPL |
| | | (SEQ ID NO:51) |
| BMS-599199 | 5-11^{b} | YPYFIPB |
| | | (SEQ ID NO:52) |
| BMS-607641 | 5-11^{d} | YPYFIPB-Alexa488 |
| | | (SEQ ID NO:108) |
| BMS-607649 | 5-11^{d} | YPBFIPL-Alexa488 |
| | | (SEQ ID NO:109) |

^{a}Peptides corresponding to the BMS-593925 sequence with an Ala mutation.
^{b}Peptides corresponding to the BMS-593925 sequence with a Bpa mutation.
^{c}Peptide containing the same amino acid as BMS-593925, but the sequence is scrambled.
^{d}Two peptides corresponding to the BMS-593925 sequence with a Bpa mutation and Alexa488 attached to the C-terminus.

### Example 9

### Characterization of EBPs Labeled at Different Positions and with Linkers of Different Lengths

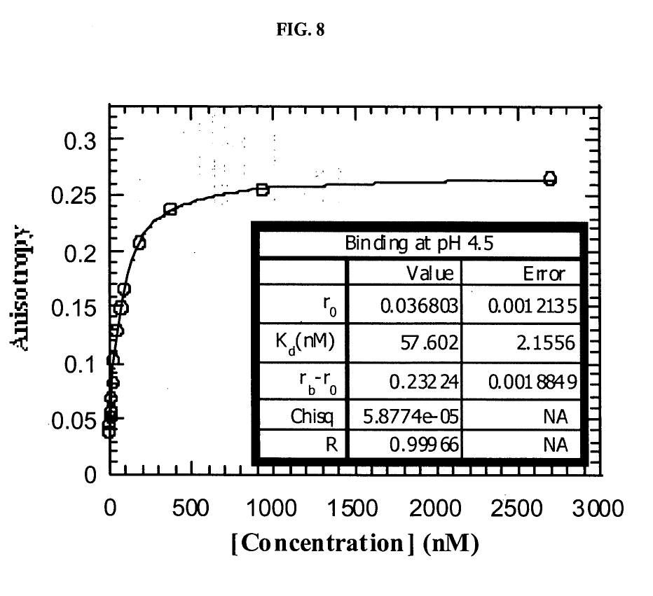

A comparison of EBPs labeled with Alexa488 at different positions (Molecules X, Y1, Z) was performed using the above binding assay. It is preferred that the labeled EBP exhibit tight binding affinity and a high signal to background ratio (i.e., ratio of the anisotropy upon binding to BACE and the anisotropy of the free EBP peptide). The binding constants of Molecules X, Y1, and Z were determined to be 903, 58, and 6430 nM, respectively. Molecule Y1 not only exhibits the tightest affinity for BACE, but also the best signal to background ratio. An example of Molecule Y1 binding to BACE is shown in FIG. 8.

Analogs of Molecule Y 1 were prepared with different length of linkers (FIG. 4). The binding constants of Y1, Y2, Y3, and Y4 were 57, 92, 48, 62 nM, respectively. The lack of change in the affinity of Molecules Yn (where n=1-4) for BACE indicates that the length of linker between Alexa488 and the peptide does not affect the strength of binding interaction.

### Example 10

### Binding of Molecule Y1 can be Displaced by Unlabeled EBP

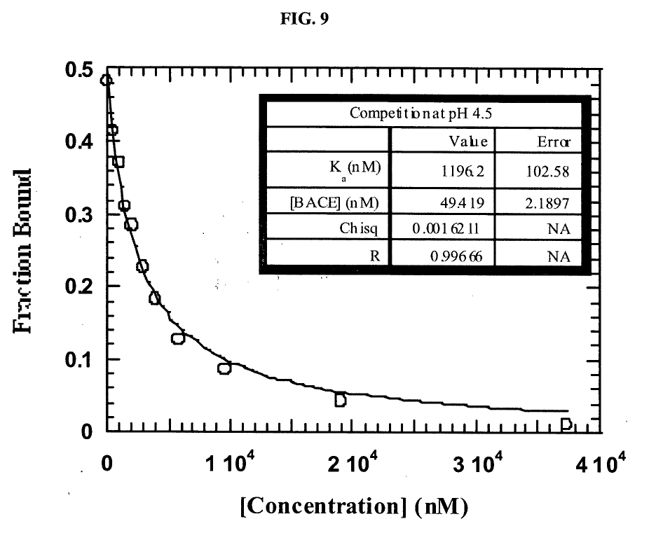

Unlabeled EBP corresponding to Molecule Y1 (BMS-593925, YPYFIPL (SEQ ID NO:10)) was used in the competition assay to displace Molecule Y1 from binding to BACE at pH 4.5 (see FIG. 9). The unlabeled EBP was found to bind to BACE with a K_{d} of 1197 nM. This demonstrates that although the presence of the C-terminal Alexa488 on Molecule Y1 increased the affinity of Y1 for BACE, it still binds to the same exosite as the unlabeled peptide since it can be displaced by the unlabeled peptide.

### Example 11

### Binding of a Labeled EBP to BACE in the Presence of an Active Site Inhibitor

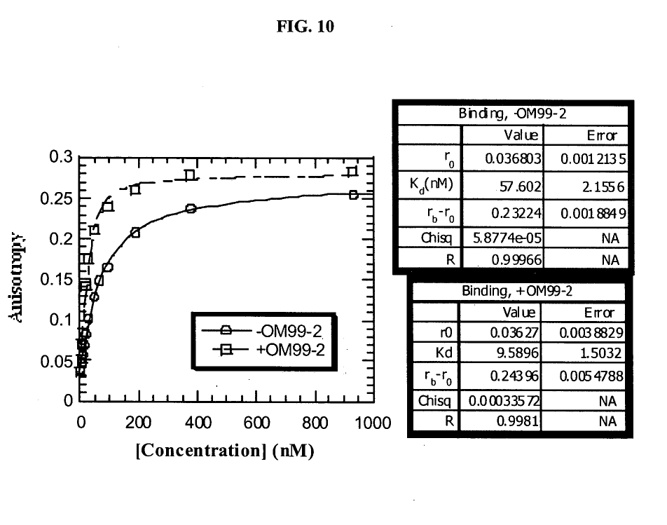

The binding assay described herein above was used to determine the binding affinity of Molecule Y1 to BACE in the presence of 10 μM OM99-2, a known BACE inhibitor that binds to BACE at the active site with a Kᵢ of 2 nM (FIG. 10). Before the addition of BACE, a concentrated stock (1 mM in 100% DMSO) of OM99-2 was mixed with the 300 μl solution of 10 nM labeled EBP (molecule Y1) to a final OM99-2 concentration of 10 μM. BACE was then titrated into this mixture of OM99-2 and labeled EBP as described above. It was found the EBP (molecule Y1) binds to BACE with somewhat enhanced affinity (5-fold) in the presence of 10 μM OM99-2. This demonstrates that the EBP does not bind to BACE at the same site as OM99-2, i.e., it binds at an exosite away from the active site. The binding of OM99-2 at the active site may have a positively cooperative effect on the EBP binding.

### Example 12

### Binding of Labeled EBP to BACE Purified from E. coli Cells

Binding of Molecule Y1 to the catalytic domain of BACE purified from E. coli cells was carried out to determine the effect of glycosylation on the EBP binding. It is known that proteins purified from E. coli do not have glycosylation. The binding affinity of E. Coli expressed human BACE for EBPs was found to be the same as that of human BACE purified from CHO cells, indicating that the EBPs are indeed binding to the BACE protein, rather than the sugar groups.

### Example 13

### Determination of the Contribution of Each Amino Acid in EBP by Ala Scan and Bpa Scan

A collection of mutated peptides base on peptide YPYFIPL (SEQ ID NO:10) was screened using the competition assay described in Example 9.

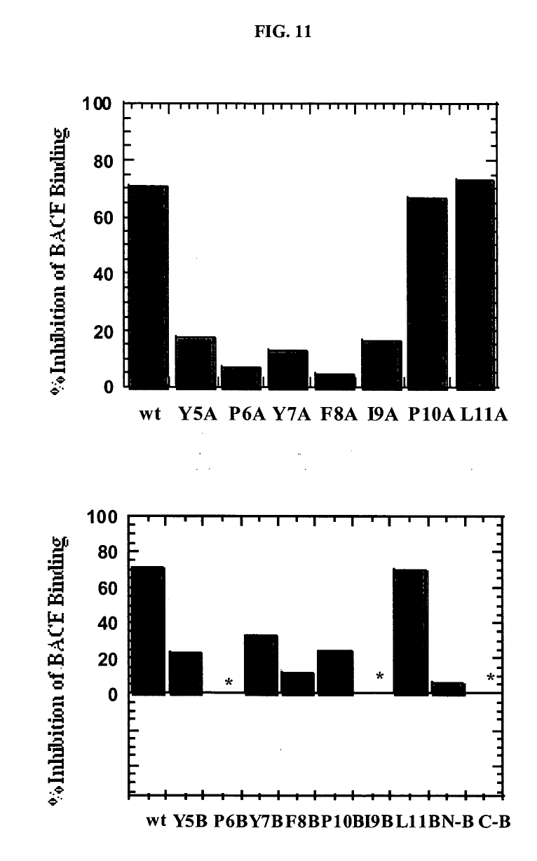

The results of the Ala scan shown in FIG. 11 (upper panel) suggests that while the last two amino acids (PL) are not critical for the binding interaction, the other five amino acids (YPYFI; SEQ ID NO:2) all play an important role in the interaction of the EBP with BACE.

The result of the scan of benzophenone-containing peptides (see FIG. 11, lower panel) suggests that Leu-11 can be replaced with a benzophenone (Bpa) group. Tyr-7 accommodates a Bpa substitution better than most other positions, but not as well as Leu-11.

### Example 14

### Photo-Crosslinking of Bpa Containing EBPs to BACE

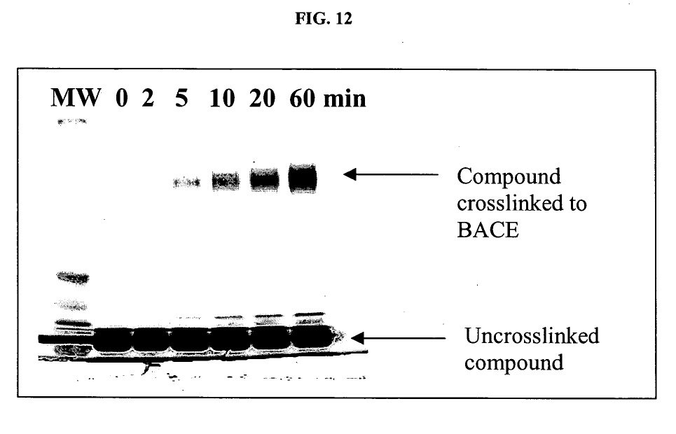

Two EBPs containing a Bpa substitution as well as an Alexa488 group attached at the C-terminus (YPYFIPB-Alexa488 (SEQ ID NO: 108) and YPBFIPL-Alexa488 (SEQ ID NO:109); where B indicates a benzophenone group) were tested for their binding to BACE. They were both found to bind to BACE reversibly with affinities around 100 nM in the absence of UV light. Both peptides were used to covalently crosslink to BACE upon UV irradiation at 360 nm. The crosslinking reaction was carried out at various temperatures in the presence of 2 μM BACE, various amounts of EBP containing the Bpa group, as well as 100 μM of a scrambled peptide with the sequence of LYPPYIF (SEQ ID NO:53) that does not bind to BACE. The reaction mixture was separated by SDS-PAGE and visualized on a Fluoroimager (Molecular Dynamics) with an excitation of 488 nm and an emission of 530 nm. FIG. 12 shows a typical time course of the crosslinking reaction of BMS-607641. The Bpa containing EBPs can be covalently crosslinked to BACE and thus serve as tools for the determination of the structure of the exosite on BACE.

### Example 15

### Assay for Identifying BACE Exosite Binding Compounds

Compounds that bind to BACE at the exosite can be discovered using the competition assay described in Examples 5 and 9. By mixing compounds at a single concentration or varying concentrations with a fixed concentration mixture of BACE and labeled EBP, changes in the fluorescence anisotropy of the Alexa488 group are followed to determine the competition (or the lack of) of compounds for the EBP binding to BACE. To facilitate the high throughput discovery of exosite binding compounds of BACE, the assay is carried out in a 96, 384, or 1536 well format.

### Example 16

### Molecule Y3 Inhibits the Proteolytic Activity of BACE

Molecule Y3 was tested in a BACE cleavage assay using the peptide MCA-EVNLDAEFK(-dnp)-COOH (SEQ ID NO:107) as a substrate. The assay was carried out essentially as described in Mallender et al., (2001) Mol. Pharmacol. 59:619-626 and in Marcinkeviciene et al., (2001) J. Biol. Chem. 276: 23790-23794. A concentration of 0.1 nM BACE was incubated with Molecule Y3 at various concentrations for 15 minutes before substrate peptide was added to a final concentration of 25 μM. The reaction was allowed to proceed for 60 minutes at 25° C. before it was stopped by boiling. The reaction mixture was separated on a C18 column using reverse phase HPLC (Waters, Milford, Mass.). The IC₅₀ value was calculated using the Langmuir isotherm equation (Copeland, R. A., Enzymes: A Practical Introduction to Structure, Mechanism, and Data Analysis, (2^{nd} ed), Wiley-VCH, New York, N.Y. (2000)).

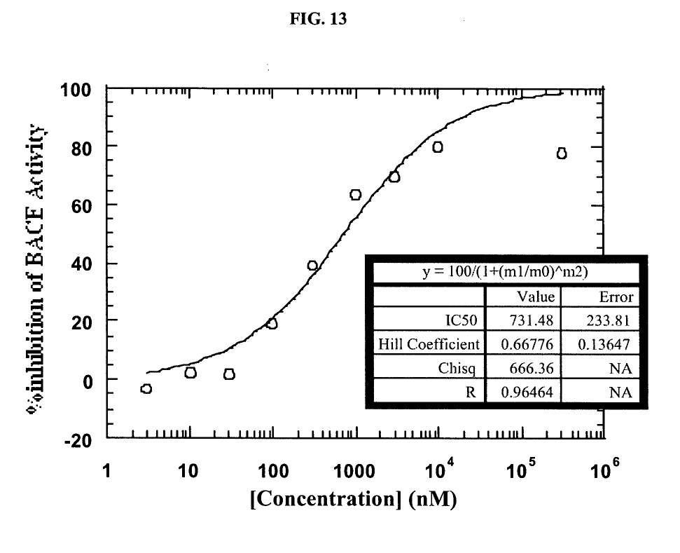

Molecule Y3 was found to inhibit the proteolytic activity of BACE with an IC₅₀ of 731 nM (FIG. 13), demonstrating that binding of EBPs to the exosite on BACE can indeed interfere with the catalytic activity of BACE. The inhibition by EBP may be more potent when protein substrates, containing an APP sequence, are used instead of short peptide substrate.

### Example 17

### Peptide Synthesis

The EBP peptides described herein were prepared using either an Applied Biosystems Inc. 433A peptide synthesizer or an Advanced Chemtech Multiple Peptide Synthesizer (MPS-396). The MPS-396 synthesizer was used to prepare several peptides simultaneously. The ABI 433A synthesizer was used to prepare individual peptides one at a time.

The syntheses of the peptide analogs described herein were also carried out either by using an Advanced Chemtech Multiple Peptide Synthesizer (MPS-396) or an Applied Biosystems Inc. peptide synthesizer. The step-wise solid phase peptide synthesis was carried out utilizing the Fmoc/t-butyl protection strategy. The amino acid derivatives used for the chain building were protected by the Fmoc group at the α-amino, and the side chain functionalities were protected by groups that are resistant to piperidine treatment, but ultimately cleavable by trifluoroacetic acid.

### Example 18

### Simultaneous Solid Phase Peptide Synthesis of EBP Peptides

4-(2′,4′-dimethoxyphenyl-Fmoc-aminomethyl)-phenoxyacetyl-p-methyl benzhydrylamine resin (Rink amide MBHA resin; loading: 0.5 mmol/g) was loaded as a suspension in dichloromethane/DMF (60:40) into the 96-well reactor of an Advanced ChemTech MPS 396 synthesizer in volumes corresponding to 0.01-0.025 mmol (20-50 mg) of resin per reactor well. The reactor was placed on the instrument and drained. The wells were then washed with DMF (0.5-1.0 mL, 3×2 min) and subjected to the number of automated coupling cycles required to assemble the respective peptide sequences as determined by the pre-programmed sequence synthesis table. The detailed stepwise synthesis protocol used for a typical 0.01 mmol/well simultaneous synthesis of 96 compounds is described below. This protocol was adapted for the simultaneous synthesis of arrays of analogs. The general synthesis protocol is depicted in Scheme 2.

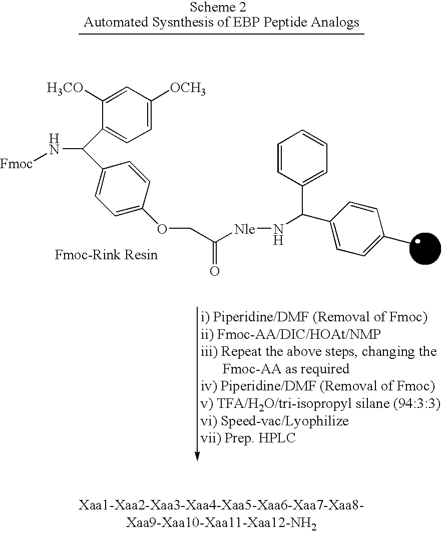

Prior to starting the synthesis, the following reagent solutions were prepared and placed on the instrument as required: 1.5 M (15%) piperidine in DMF; 0.5 M DIEA in NMP; 0.36 M DIC in NMP; 1 M (10%) acetic anhydride in DMF. The required Fmoc-protected amino acids were prepared as 0.36 M solutions in 0.36 M HOAt/NMP and placed into the appropriate positions in the 32-position amino acid rack.

Coupling of the amino acid residue was carried out by automated addition of a 0.36 M solution of the appropriate Fmoc-amino acid (0.072 mmol, 7.2 eq.) and HOAt (7.2 eq.) in NMP (0.2 mL) to all relevant wells. This was followed by addition of a 0.36 M solution of DIC (0.072 mmol, 7.2 eq.) in NMP (0.2 mL). The coupling was allowed to proceed for 2 hrs. After reactor draining by nitrogen pressure (3-5 psi) and washing the wells with NMP (1×0.5 mL), the coupling was repeated as described above. At the end of the coupling cycle, the wells were treated with 1M acetic anhydride in DMF (1×0.5 mL, 30 min.) and finally washed with DMF (3×0.5 mL).

An identical coupling protocol was repeated additional times in order to complete the sequence assembly of the desired peptide analogs.

Finally, the Fmoc group was removed with 20% piperidine in DMF as described above, and the peptidyl-resins were washed with DMF (4×0.5 mL) and DCM (4×0.5 mL). They were then dried on the reactor block by applying a constant pressure of nitrogen gas (5 psi) for 10-15 min.

### Cleavage/Deprotection

The desired peptides were cleaved/deprotected from their respective peptidyl-resins by treatment with a TFA cleavage mixture as follows. A solution of TFA/water/tri-isopropylsilane (94:3:3) (1.0 mL) was added to each well in the reactor block, which was then vortexed for 2 hrs. The TFA solutions from the wells were collected by positive pressure into pre-tared vials located in a matching 96-vial block on the bottom of the reactor. The resins in the wells were rinsed twice with an additional 0.5 mL of TFA cocktail and the rinses were combined with the solutions in the vials. These were dried in a SpeedVac™ (Savant) to yield the crude peptides, typically in >100% yields (20-40 mgs). The crude peptides were either washed with ether or more frequently re-dissolved directly in 2 mL of DMSO or 50% aqueous acetic acid for purification by preparative HPLC as follows.

### Preparative HPLC Purification of the Crude Peptides

Preparative HPLC was carried out either on a Waters Model 4000 or a Shimadzu Model LC-8A liquid chromatograph. Each solution of crude peptide was injected into a YMC S5 ODS (20×100 mm) column and eluted using a linear gradient of MeCN in water, both buffered with 0.1% TFA. The desired product eluted well separated from impurities, typically after 8-10 min., and was collected in a single 10-15 mL fraction on a fraction collector. The desired peptides were obtained as amorphous white powders by lyophilization of their HPLC fractions.

### HPLC Analysis of the Purified Peptides

After purification by preparative HPLC as described above, each peptide was analyzed by analytical RP-HPLC on a Shimadzu LC-10AD or LC-10AT analytical HPLC system consisting of: a SCL-10A system controller, a SIL-10A auto-injector, a SPD10AV or SPD-M6A UV/VIS detector, or a SPD-M10A diode array detector. A YMC ODS S3 (4.6×50 mm) column was used and elution was performed using a linear gradient of MeCN in water, both buffered with 0.1% TFA. Mobile phase A: 0.1% TFA/water; mobile phase B: 0.1% TFA/acetonitrile. The purity was typically >90%.

### Characterization by Mass Spectrometry

Each peptide was characterized by electrospray mass spectrometry (ES-MS) either in flow injection or LC/MS mode. Finnigan SSQ7000 single quadrupole mass spectrometers (ThermoFinnigan, San Jose, Calif.) were used in all analyses in positive and negative ion electrospray mode. Full scan data was acquired over the mass range of 300 to 2200 amu for a scan time of 1.0 second. The quadrupole was operated at unit resolution. For flow injection analyses, the mass spectrometer was interfaced to a Waters 616 HPLC pump (Waters Corp., Milford, Mass.) and equipped with an HTS PAL autosampler (CTC Analytics, Zwingen, Switzerland). Samples were injected into a mobile phase containing 50:50 water:acetonitrile with 0.1% ammonium hydroxide. The flow rate for the analyses was 0.42 mL/min. and the injection volume 6 μL. A ThermoSeparations Constametric 3500 liquid chromatograph (ThermoSeparation Products, San Jose, Calif.) and HTS PAL autosampler were used for LC/MS analyses. Chromatographic separations were achieved employing a Luna C₁₈, 5 micron column, 2×30 mm (Phenomenex, Torrance, Calif.). The flow rate for the analyses was 1.0 mL/min and column effluent was split, so that the flow into the electrospray interface was 400 μL/min. A linear gradient from 0% to 100% B in A over 4 minutes was run, where mobile phase A was 98:2 water:acetonitrile with 10 mM ammonium acetate and mobile phase B was 10:90 water:acetonitrile with 10 mM ammonium acetate. The UV response was monitored at 220 nm. The samples were dissolved in 200 μL 50:50 H₂O:MeCN (0.05% TFA). The injection volume was 5 μl.

In all cases, the experimentally measured molecular weight was within 0.5 Daltons of the calculated mono-isotopic molecular weight.

### Example 19

### Solid Phase Synthesis of EBP Peptide Analogs Using an Applied Biosystems Model 431 A Peptide Synthesizer

Following is the general description for the solid phase synthesis of typical EBP peptide analogs, using an upgraded Applied Biosystems Model 433A peptide synthesizer. The upgraded hardware and software of the synthesizer enabled conductivity monitoring of the Fmoc deprotection step with feedback control of coupling. The protocols allowed a range of synthesis scale from 0.05 to 0.25 mmol.

4-(2′,4′-dimethoxyphenyl-Fmoc-aminomethyl)-phenoxyacetyl-p-methyl benzhydrylamine resin (Rink amide MBHA resin; loading: 0.5 mmol/g) (0.1 mmol) was placed into a vessel of appropriate size on the instrument, washed 6 times with NMP and deprotected using two treatments with 22% piperidine/NMP (2 and 8 min. each). One or two additional monitored deprotection steps were performed until the conditions of the monitoring option were satisfied (<10% difference between the last two conductivity-based deprotection peaks). The total deprotection time was 10-12 min. The first Fmoc-protected amino acid was coupled next using the following method: Fmoc-AA-OH (1 mmol, 10 eq.) was dissolved in 2 mL of NMP and activated by subsequent addition of 0.45 M HBTU/HOBt in DMF (2.2 mL) and 2 M DIEA/NMP (1 mL). The solution of the activated Fmoc-protected amino acid was then transferred to the reaction vessel and the coupling was allowed to proceed for 30 to 60 min., depending on the feedback from the deprotection steps. The resin was then washed 6 times with NMP, and subjected to the additional deprotection/coupling cycles as described above necessary to complete the assembly of the desired sequence. Finally, the Fmoc group was removed with 22% piperidine in NMP as described above, and the peptidyl-resin was washed 6 times with NMP and DCM, and dried in vacuo.

### Cleavage/Deprotection

The desired peptide was cleaved/deprotected from its respective peptidyl-resin by treatment with a solution of TFA/water/tri-isopropylsilane (94:3:3) (5.0 mL/g of peptidyl-resin) for 2 hrs. The resin was filtered off, rinsed with TFA cleavage solution (2 mL), and the combined TFA filtrates were dried in vacuo. The resulting solid was triturated and washed with diethyl ether, and finally dried, to yield the crude peptide product as a white solid. This was purified by preparative HPLC as described herein. The fraction containing a pure product was lyophilized, to yield the pure peptide product in 20-40% isolated yield.

### Example 20

### Coupling of the Alexa488 Label to a EBP Peptide

The Alexa488 label was attached to either the α-amino group of the N-terminal amino acid residue of a EBP peptide or the camino group of the side chain of a α,ω-diamino acid appended to the C-terminus of a EBP peptide by reaction of the purified EBP peptide with the N-hydroxysuccinimidyl ester of the Alexa Fluor® 488 fluorophore [1.5-2.0 eq.] for 16-20 hrs in NMP and DIEA (1-2 eq). The reaction progress was monitored by HPLC. The resulting Alexa488-labeled EBP peptide was then purified by HPLC and characterized as described herein.

### Example 21

### Biased Library Peptides Identified by Solution Panning at pH 5.2

Panning was performed at pH 5.2 to identify peptides that bind to the exosite under these conditions more tightly than was the case for the peptides derived from the unbiased libraries. The methods employed are identical to those described in Example 2, with the exception that Protein A cells were replaced by Protein A agarose beads (Sigma, St. Louis, Mo.) and that amounts of BACE, number of washes and temperatures of wash buffers were used as outlined below to maximize recovery of the tightest binding phage. More particularly, two biased M13-based peptide libraries were panned against BACE-Ig prepared from CHO cells, described herein above. Protein A beads were preblocked in 50 mM NaOAc, pH 5.2, +2% BSA for 2 hours. In parallel, BACE was incubated for two hours with library phage using the same buffer. Both samples were then mixed together for two hours. This step was followed by several washes and phage were eluted with 6M urea, pH 3.0, and used for amplification and further panning cycles or DNA sequencing after round three. Cycle 1:10 micrograms of BACE were used and 6 quick washes were carried out with PBS plus 0.2% Tween 20 at room. temperature. Cycle 2:50 nanograms of BACE were used and there were 7 washes of 3 minutes duration each with 50 mM NaOAc, pH 5.2, +0.2% Tween 20 at 37° C. Cycle 3:25 nanograms of BACE were used and there were 15 washes of three minutes duration each using 0.3M NaOAc, pH 5.2, at 37° C.

Biased peptide libraries were employed in this Example. The biased libraries were made as described herein (see also, Sidhu et al., (2000) Method Enzymol. 328:333-363), except that the residues defining the core motif (i.e., HYPYFI (SEQ ID NO:54) were fixed in order to bias the peptides. Each X corresponds to one random library residue. All peptides in the table below are preferably synthesized with an added unblocked N-terminal Ala, while C-termini are preferably blocked. The libraries were designed as follows:

| | | |
|---|---|---|
| linear | XXXXXHYPYFIXXXXX | (SEQ ID NO:55) |
| library 1: | | |
| cyclic | CysXXXXXHYPYFIXXXXXCys | (SEQ ID NO:56) |
| library 2: | | |

Peptides observed to bind to the BACE exosite at pH 5.2 are shown in the table presented below. In the table, the fixed motif is indicated by italics. Peptides are grouped into sets with shared sequence similarity within the random segments and those similarities are in bold. Potential disulfide bonds are indicated by underlining. 6 peptides gave the most improved phage ELISA binding signal relative to BMS-561871 on phage and are identified in the table by “XXX”. The peptides marked XXX are all cyclic, one of which has a third internal Cys. Although it is not the inventors' desire to be bound to any theory of operation, it is noted that the peptide with the internal Cys may be interesting in a scenario in which the peptides bind through the fixed core motif and then sterically interfere with the access of substrate to the active site. In this case, it may be that this peptide, and others like it, are better inhibitors compared to other peptides that exhibit the same affinity.

TABLE 4

Exosite Binding Peptides Identified by Solution
Panning at pH 5.2

| | | |
|---|---|---|
| TDQPKHYPYFIPSPHS | SEQ ID NO:58 | |
| THQPKHYPYFIPYHHD | SEQ ID NO:59 | |
| MDHEKHYPYFIEYKHV | SEQ ID NO:60 | |
| CTEANKHYPYFIPRHSSC | SEQ ID NO:61 | |
| HSLAPHYPYFIDLHST | SEQ ID NO:62 | |
| GSQALHYPYFIPYHKH | SEQ ID NO:63 | |
| CTNKHDHYPYFIRPGEFC | SEQ ID NO:64 | |
| CENKHDHYPYFISAGNYC | SEQ ID NO:65 | |
| CQTKVMHYPYFIREGVTC | SEQ ID NO:66 | |
| CGPKHLHYPYFISATSRC | SEQ ID NO:67 | XXX |
| CAAKHSHYPYFIPACSSC | SEQ ID NO:68 | |
| CASTYPHYPYFIATCKTC | SEQ ID NO:69 | |
| CAEAKQHYPYFIKWCKTC | SEQ ID NO:70 | |
| CAEAKGHYPYFICTTGNC | SEQ ID NO:71 | |
| CAQAREHYPYFIDLRTV | SEQ ID NO:72 | |
| CAKAPRHYPYFISAQNAW | SEQ ID NO:73 | |
| CAKASHHYPYFINLANNG | SEQ ID NO:74 | |
| CARAITHYPYFIPYCEEC | SEQ ID NO:75 | XXX |
| AVSQTHYPYFIPLSQA | SEQ ID NO:76 | |
| CEDRPTHYPYFISLNKQC | SEQ ID NO:77 | |
| CKTQDNHYPYFISLKKAC | SEQ ID NO:78 | |
| CQTKHQHYPYFISLTDAC | SEQ ID NO:79 | XXX |
| CTKAHTHYPYFISNSKIC | SEQ ID NO:80 | |
| CHHKHTHYPYFIPNTKSC | SEQ ID NO:81 | |
| CSQHHTHYPYFIPSNGMC | SEQ ID NO:82 | XXX |
| CAVEARHYPYFINTCSNC | SEQ ID NO:83 | |
| CSVVNRHYPYFINNSSKC | SEQ ID NO:84 | |
| CTGCARHYPYFIEVSTQW | SEQ ID NO:85 | |
| CSNASHHYPYFISTHSTC | SEQ ID NO:86 | |
| CSNPTGHYPYFISPQGTC | SEQ ID NO:87 | |
| CNSTPRHYPYFISVNSTC | SEQ ID NO:88 | |
| CGVQLVHYPYFLPANSTC | SEQ ID NO:89 | |
| CARTPSHYPYFISLPDRG | SEQ ID NO:90 | |
| CSAGHNHYPYFITLPGYG | SEQ ID NO:91 | |
| CASQDYHYPYFIPSPAWG | SEQ ID NO:92 | |
| ELPFQHYPYFIDLPPV | SEQ ID NO:93 | |
| MHPNPHYPYFIPLPTR | SEQ ID NO:94 | |
| CDSCVTHYPYFINTPYKY | SEQ ID NO:95 | |
| CAKPKQHYPYFICYPHEC | SEQ ID NO:96 | |
| INKTQHYPYFIEYPFH | SEQ ID NO:97 | |
| CPNTQHHYPYFIKVGEHC | SEQ ID NO:98 | XXX |
| CPDIAHHYPYFIDSKSHC | SEQ ID NO:99 | |
| CQPTRHHYPYFIDVTGRC | SEQ ID NO:100 | |
| CQNNHHHYPYFITPTHVC | SEQ ID NO:101 | |
| CTTTHEHYPYFIDPREAC | SEQ ID NO:102 | XXX |
| CTTPSRHYPYFIDQLGHC | SEQ ID NO:103 | |
| CNANHTHYPYFIDISRKC | SEQ ID NO:104 | |
| QFTHKHYPYFINISPG | SEQ ID NO:105 | |
| CNMPHSHYPYFINPHQSC | SEQ ID NO:106 | |

### Example 22

### BACE Exosite Binding Studies of Peptide BMS-655507 BACE Protein Preparation

BACE samples were prepared for isothermal titration calorimetry by extensive dialysis against freshly prepared buffer at 4° C. using 12,000-14,000 kDa MWCO dialysis membrane (UltraPURE, GIBCO BRL). The buffers used for these experiments were either Dulbecco's PBS (2 mM KH₂PO₄, 8 mM Na₂HPO₄, 137 mM NaCl, 3 mM KCl), pH 7.0, or 50 mM NaOAc, pH 4.5. The pH values were determined at room temperature. Following two changes of buffer (500 mL each), the protein (typically 2-3 mL) was removed from the membrane and centrifuged (5 min×4000 g, 4° C.) to remove particulates. Following dialysis, the dialysate was filtered (0.22 μm) and retained for preparation of the peptide samples (below) and rinsing the sample cell of the calorimeter between experiments. The protein was stored at 4° C. until needed for the calorimetry experiments. The protein concentration was determined by 10-fold dilution into the same buffer, and measuring the UV absorbance at 280 nm in a 1.00 cm pathlength cell (calculated value=1.22 au=1.00 mg/mL protein, based on mean glycosylated molecular weight=53.5 kDa, and given amino acid composition). Final concentrations used in isothermal titration calorimetry were typically 4-5 μM, containing a final concentration of 1% v/v DMSO.

### Preparation of Peptide, BMS-655507

The peptide BMS-655507 (Ac-His-Trp-Pro-Phe-Phe-Ile-Arg-Ser; SEQ ID NO:57) was dissolved in the buffer dialysate, and a volume of DMSO added to yield 1.0% v/v. The pH of the peptide solutions were adjusted as necessary to equal that of the buffer dialysate and protein sample (within 0.01 pH units). The concentrations of peptide were determined measuring the UV absorbance at 280 nm, using the molar extinction coefficient value of E=5630 M⁻ cm⁻¹.

### Isothermal Titration Calorimetry

Isothermal titration calorimetry experiments were performed with a VP-ITC instrument from MicroCal Inc. (Northampton, Mass.). The instrument was controlled with a personal computer, and thermally regulated at the desired experimental temperature (25° C.). Samples of BACE and peptides were degassed for 15 min at room temperature using a degassing unit (MicroCal) before loading into the sample chamber or syringe, respectively. Deionized, degassed water was loaded into the instrument reference chamber and used for all experiments. For each titration experiment, a fresh sample of BACE (typically 5 μM, 2.0 mL) was loaded into the instrument sample chamber (volume=1.438 mL), using a glass syringe, following the manufacturer's directions. Similarly, fresh peptide samples (typically 130-180 μM for BMS-655507, 0.3 mL total volume) were loaded into the instrument injecting syringe unit before each experiment.

### Instrument Parameters

The temperature was maintained at 25° C. during the titration experiments. A power setting of 6.0 μCal/sec was used, and a syringe stirring rate of 300 rpm was used. The initial injection was kept at 1.5-2.0 μL and the data from this injection was not included in the analysis as a standard practice. To completely define the binding isotherm, typically a 3-fold to 4-fold excess of peptide was added during the course of the titration experiment, using ˜8 injections per molar equivalent, or 4-7 μL (BMS-655507) per injection. The data collection time per injection was fixed at 360 sec, with a signal averaging time of 2 sec. The data was analyzed using the manufacturer's software (i.e. Origin 5.0 for ITC). Before molar heat calculations were done, background corrections were made on all peaks by subtracting the mean of the final 8-12 injections from all injections.

The calculated molar heat values were fitted to a single binding site model using the manufacturer's software to determine the binding stoichiometry (n), the association constant (K_{A}), the enthalpy of the reaction (AH), and the entropy of the reaction (ΔS). These values were used to calculate the dissociation constant (K_{d}) which is the reciprocal of K_{A}, and the Gibbs free energy of the reaction (ΔG), which is related to the K_{A}, ΔH, and ΔS by the following equations: ΔG=−RT (ln(K_{A}))=ΔH−TΔS (Levine, Physical Chemistry, (2^{nd} ed.), McGraw-Hill Co. (1983), p. 125).

The sample cell was cleaned between injections by washing extensively with filtered PBS or NaOAc buffer, H₂O, and again with filtered buffer. After multiple experiments, the sample cell and syringe were more extensively cleaned using manufacturer's recommendations with a detergent solution heated to 50° C., followed by extensive washing with H₂O, methanol, H₂O, and finally filtered buffer. Blank injections of buffer into buffer: were then performed to establish sufficient cleaning and reproducible background before carrying out additional BACE-peptide experiments.

### Results

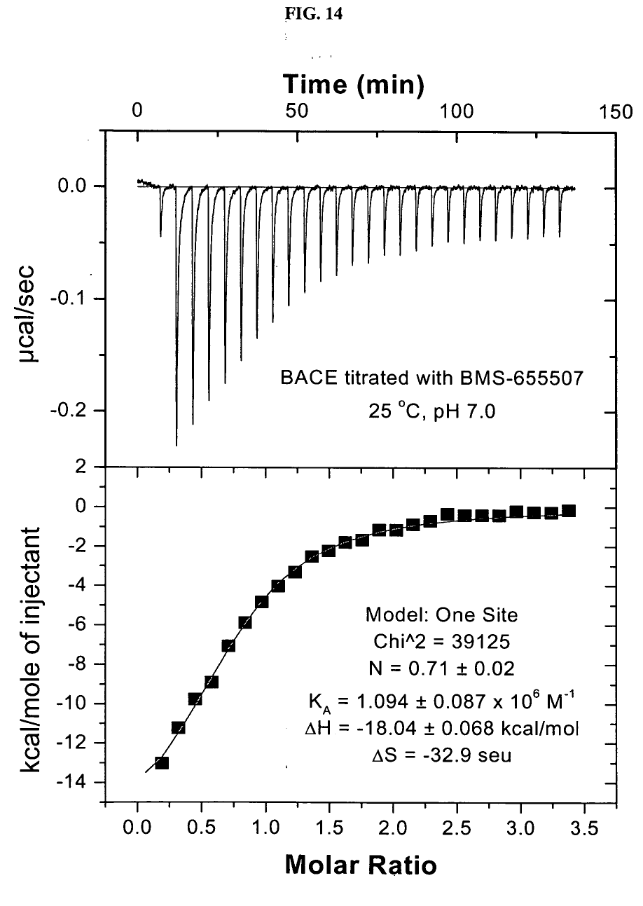

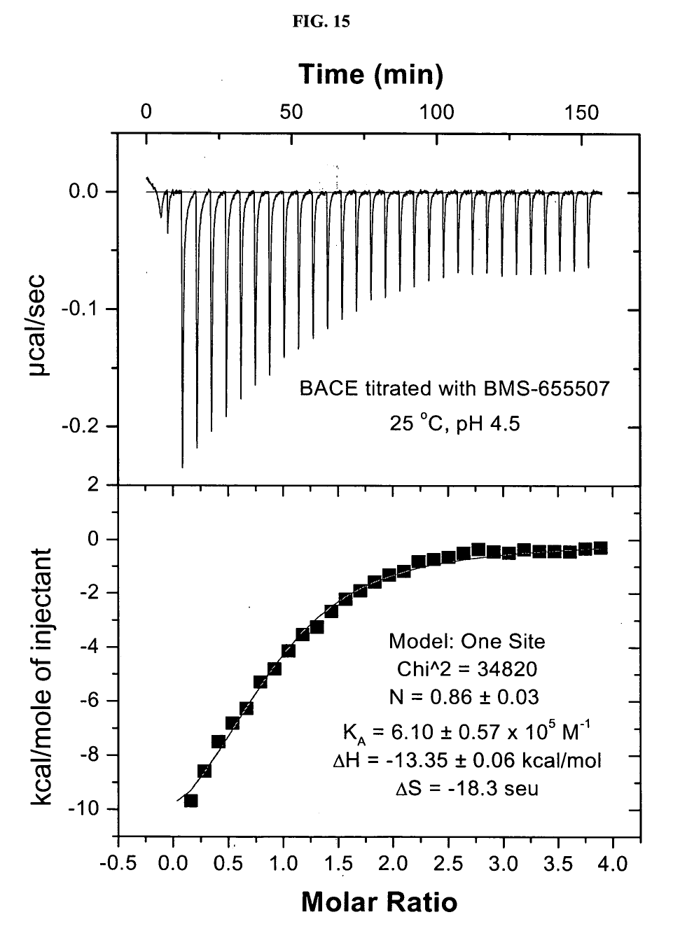

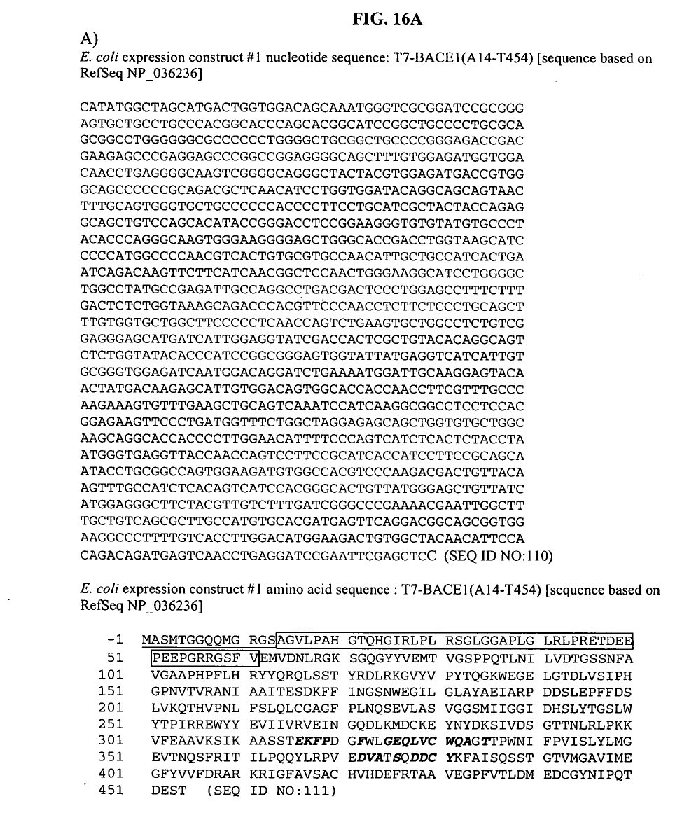

Similar to previous experiments with BMS-561871 and BMS-561877, titrations of BMS-655507 into a solution containing BACE demonstrated saturable 1:1 binding. Experiments were completed in both Dulbecco's PBS, pH 7.0 and 50 mM NaOAc, pH 4.5, at 25° C. (FIGS. 14 and 15). The binding constants were determined to be K_{d}=0.914 μM for at pH 7.0 and K_{d}=1.64 μM at pH pH 4.5, as summarized in the following table.

TABLE 5
Calculated and Fitted Thermodynamic Data for Experiments with
BACE and BMS-655507 Conducted at pH 4.5 And 7.0, 25° C.

| pH | ΔG | ΔH | TΔS | K_{d} | |
|---|---|---|---|---|---|
| value | (kcal/mol) | (kcal/mol) | (kcal/mol) | (μM) | Stoichiometry |
| pH 4.5 | −7.90 | −13.35 | −5.46 | 1.64 | 0.86 |
| pH 7.0 | −8.23 | −18.04 | −9.81 | 0.914 | 0.71 |

### Example 23

### BACE-1 Protein Production and Measurement of Activity for X-ray Crystallography

BACE-1 Constructs for X-Ray Crystallography were Produced and Purified as Follows:

Construct #1: BACE-1 (T7-BACE1 (A14-T454)):

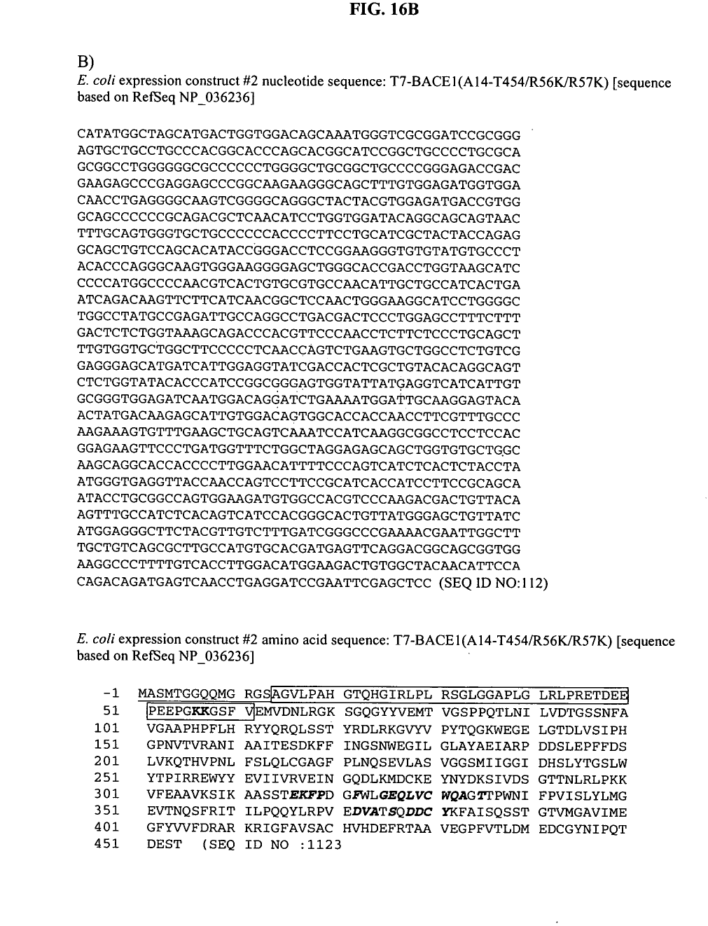

A cDNA fragment encoding an N-terminal T7 tag followed by BACE-1 residues 14-454 (numbering based on GenBank Accession No. NP_{—}036236) was cloned into pET21a for expression (FIG. 16A). BACE-1 expression, refolding and purification was based on the procedure of Hong et. al. (Hong et. al., (2000) Science, 290:150-153). Recombinant BACE-1 was over-expressed in Luria broth (LB) as inclusion bodies in E. Coli Rosetta BL21(DE3) cells. The E. coli cell paste was re-suspended in TN buffer (100 mM Tris pH 7.4, 150 mM NaCl) plus 1 mg/ml lysozyme and stirred for 30 minutes at 24° C. 10 mM MgCl₂ and DNase I were added and the solution was stirred for an additional 30 minutes. The suspension was homogenized with an Emulsiflex (Avestin) homogenizer followed by the addition of Triton X-100 to 0.1%.

The whole cell homogenate was then stirred for 1 hour and centrifuged to yield insoluble inclusion bodies after supernatant decantation. The inclusion bodies were washed 4 times with TN buffer plus 0.1% Triton X-100 then 2 times with TN buffer to yield a homogeneous pellet. The inclusion body pellet was solubilized with 23 mls denaturing buffer (8M Urea, 10 mM Tris pH 10.0, 1 mM EDTA, 1 mM Glycine) and 10 mM β-mercaptoethanol (BME) per gram of inclusion body pellet. The mixture was then stirred for 1 hour at 24° C. Insoluble particulates were removed by centrifugation and the supernatant was diluted to an OD₂₈₀ of 0.5 with denaturing buffer containing 0.1 mM oxidized glutathione, 1.0 mM reduced glutathione, 10 mM di-thiothreitol (DTT) and 10 mM BME. The pH was adjusted to 10.0 with 1M NaOH. 250 ml of this denatured protein solution was added drop wise to 4 L of rapidly stirring refolding buffer (20 mM Tris pH 9.0). Afterwards, the pH was adjusted to 9.0 with 1M HCl and stirred slowly at 4° C. After 18 hours, the pH was adjusted to 8.5, readjusted 24 hours later to 8.0 and then stirred for 3 weeks. The refolded protein mixture was filtered to remove particulates, loaded onto RESOURCE Q resin (GE Healthcare, Inc.) previously equilibrated with buffer A (50 mM Tris pH 8.0, 0.4M Urea) and eluted with buffer A plus 0.7M NaCl.

The pooled fractions containing active protein were then concentrated and loaded onto a Sephacryl S-100 column equilibrated with 20 mM Tris pH 8.0, 0.4M Urea. The BACE containing fractions were pooled and then further purified by repeating the first purification step using the RESOURCE Q resin. Fractions of major peaks were pooled and dialyzed against 20 mM NaAcetate, pH 4.4 at 4° C. The protein sample was concentrated to 3 mg/ml, flash frozen in liquid nitrogen and stored at −80° C.

Construct #2: BACE-1 (T7-BACE1(A14-T454/R56K/R57K)):

A cDNA fragment encoding an N-terminal T7 tag followed by BACE-1 residues 14-454 (numbering based on GenBank Accession No. NP_{—}036236) was cloned into pET21a for expression (FIG. 16B). BACE-1 mutagenesis, expression, refolding and purification was based on the procedure of Patel et al. (Patel et. al. (2004) J. Mol. Biol. 343:407-416). Mutagenesis of residues R56 and R57 was performed using the Gene Tailor Mutagenesis Kit (Stratagene). Recombinant BACE-1 double-mutant was over-expressed in Overnight Express Instant TB media (Novagen) as inclusion bodies in E. Coli Rosetta 2 (DE3) cells. Inclusion bodies were prepared as described above. Insoluble particulates were removed by centrifugation. The supernatant (˜100 ml) was diluted to 900 ml with 8M Urea, 0.2 mM oxidized glutathione and 1.0 mM reduced glutathione.

250 ml of this denatured protein solution was then added to 4 L of rapidly stirring refolding buffer (20 mM Tris pH 9.0, 10 mM NDSB-256 (Calbiochem)) and stirred slowly at 4° C. After 18 hours, the pH was adjusted to 9.0 with 1M HCl and stirred for 3 weeks. The pH of the refolded protein mixture was adjusted to pH 8.0, filtered to remove particulates and loaded overnight onto a SOURCE Q resin (GE Healthcare) equilibrated with buffer A (50 mM Tris pH 8.0, 0.4M Urea). The protein was eluted from the SOURCE Q resin by a step gradient using buffer A plus 0.7M NaCl. Pooled fractions containing active protein were cleaved with clostripain (Sigma) to produce protein with a homogeneous N-terminus. The reaction mixture was reloaded onto SOURCE: Q resin and eluted as described above. Fractions containing cleaved protein were concentrated and loaded onto a Sephacryl S-100 column equilibrated with 20 mM Tris pH 8.0, 0.4M Urea. Fractions of major peaks were pooled and submitted for mass spectral analysis to confirm complete conversion to the cleaved form of the protein. The active protein was dialyzed into 50 mM Tris pH 8.5, 150 mM NaCl, concentrated to 7 mg/ml, flash frozen in liquid nitrogen and stored at −80° C.

Measurement of BACE-1 Activity:

A Fluorescence Resonance Energy Transfer Assay was then utilized to measure the activity of BACE-1. In order to do so, 20 ml of BACE-1 refolding solution was concentrated to approximately 200 μls. 10 ul of solution was then mixed with 185 ul of assay buffer (50 mM Sodium Acetate pH 4.5, 0.25 mg/ml BSA) and 5 ul of MCA peptide (Sigma, A-1222) at 25° C. Fluorescence was measured as a function of time using 330 nm excitation and 400 nm emittance wavelengths.

### Example 24

### Crystallization and Exosite Peptide Soaking

BACE-1 (T7-BACE1(A14-T454)) Complexed with Active Site Inhibitor DPH-153979 and Exosite Peptide BMS-597041:

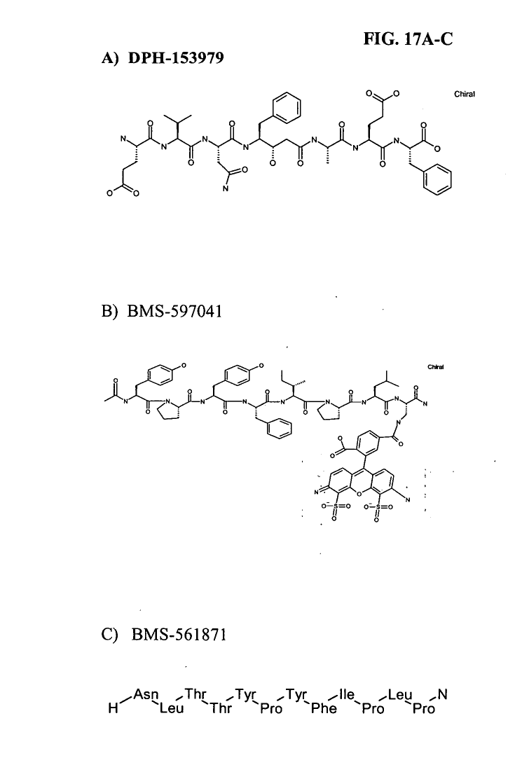

An aliquot of protein was thawed and incubated with a 10-fold molar excess of active site inhibitor DPH-153979 (FIG. 17A) for 4-5 hours at 20° C. Crystals of BACE-1 complexed with DPH-153979 were grown using the hanging drop vapor diffusion method at 20° C. 4 μl protein were mixed with 4 μl of reservoir solution consisting of 16% PEG 8000, 0.1M Na Cacodylate pH 6.2, and 0.2M Ammonium Sulfate. On day 4, streak seeding was employed to generate large single diffraction quality crystals. Crystals appeared overnight and grew to their final dimensions (0.4×0.3×0.2 mm) in about two weeks. The exosite peptide, BMS-597041 (FIG. 17B), was soaked into the crystal by transferring a single BACE-1/DPH-153979 co-crystal into a drop containing 35% PEG 8000, 0.1M Na Cacodylate pH 6.2, 0.2M Ammonium Sulfate, 0.5 mM DPH-153979 and 1 mM BMS-597041. After 4 days, the crystal was transferred into cryoprotectant (35% PEG 8000, 0.1M Na Cacodylate pH 6.2, 0.2M Ammonium Sulfate, 0.5 mM DPH-153979 and 1 mM BMS-597041 plus 20% glycerol) and flash cooled in liquid nitrogen prior to data collection.

BACE-1 (T7-BACE1(A14-T454/R56K/R57K)) Complexed with Active Site Inhibitor DPH-153979 and Exosite Peptide BMS-561871:

An aliquot of protein was thawed and incubated with 8.5% DMSO and 1.4 mM DPH-153979 (FIG. 17A) overnight at 4° C. Crystals of BACE-1 complexed with DPH-153979 were grown using the hanging drop vapor diffusion method at 24° C. 1 μl of protein was mixed with 1 μl of reservoir solution consisting of 10% ME PEG 5000, 9.5% PEG 8000, 0.2M Ammonium Iodide, and 0.1M Sodium Citrate, pH 6.4. Macro seeding was employed to generate large single diffraction quality crystals. Crystals appeared within a few days after seeding and grew to their final dimensions (0.3×0.2×0.2 mm) in about two weeks. The exosite peptide, BMS-561871 (FIG. 17C), was soaked into the crystal step wise by adding, directly to the crystal drop, a stabilizing solution of 18% ME PEG 5000, 22% PEG 8000, 0.2M Ammonium Iodide, 0.1M Sodium Citrate, pH 6.4 and 1 mM BMS-561871 to achieve an exosite concentration of 0.5 mM, followed by adding stabilizing solution and 2 mM BMS-561871 to achieve an exosite concentration of 1 mM and then adding stabilizing solution and 4 mM BMS-561871 to achieve a 1.75 mM final concentration. After 5 hours, the crystal was transferred into cryoprotectant (18% ME PEG 5000, 22% PEG 8000, 0.2M Ammonium Iodide, 0.1M Sodium Citrate, pH 6.4, 1 mM BMS-561871 and 20% glycerol) and flash cooled in liquid nitrogen prior to data collection.

### Example 25

### Structure Determination and Refinement

BACE-1 (T7-BACE1(A14-T454)) Complexed with Active Site Inhibitor DPH-153979 and Exosite Peptide BMS-597041:

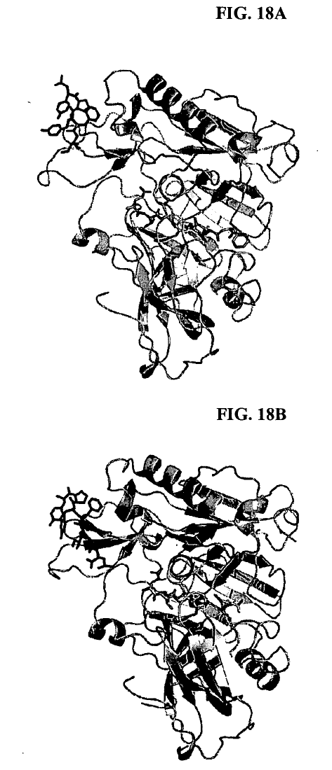

Data were collected at 100° K using a Rigaku R-axis II system mounted on a RU-200 rotating anode generator and processed using HKL2000 (Otwinowski et. al., (1997) Methods in Enzymology, Macromolecular Crystallography, Part A; C. W. Carter, Jr. and R. M. Sweet, Eds.; Academic Press: New York, 276:307-326) (Table 6). The structure was determined by molecular replacement using the program EPMR (Kissinger et. al. (1999) Acta Crystallog. Sect. D, 55:484-491) in space group P2₁ with one BACE dimer per asymmetric unit. The starting model used was a BACE dimer (without inhibitor) from a previously determined structure. Strong, unambiguous electron density was observed in the initial maps for active site inhibitor DPH-153979 for both molecules in the asymmetric unit. The next highest peaks of unassigned density were identified near residues 316-331 (Table 9) for both molecules. The active site inhibitor DPH-153979 was built into the electron density using the program QUANTA (Accelrys Software, Inc.) and refinement was carried out using the program CNX (Accelrys Software, Inc.). Subsequent electron density maps confirmed the location of the exosite peptide and allowed for the preliminary building of the core sequence YPYFI of BMS-597041 into the exosite of BACE-1 (FIG. 18A). Refinement was not carried out past initial building of YPYFI due to ambiguous exosite peptide density. Table 7 lists the coordinates of T7-BACE1(A14-T454) complexed with DPH-153979 and BMS-597041 and Table 9 shows the correlation of the amino acid sequence numbering between BACE-1 (GenBank Accession No. NP-036236) and Hong et. al. (Hong et. al., (2000) Science, 290:150-153) for residues within 6 Å of exosite peptide BMS-561871.

BACE-1 (T7-BACE1(A14-T454/R56K/R57K)) Complexed with Active Site Inhibitor DPH-153979 and Exosite Peptide BMS-561871:

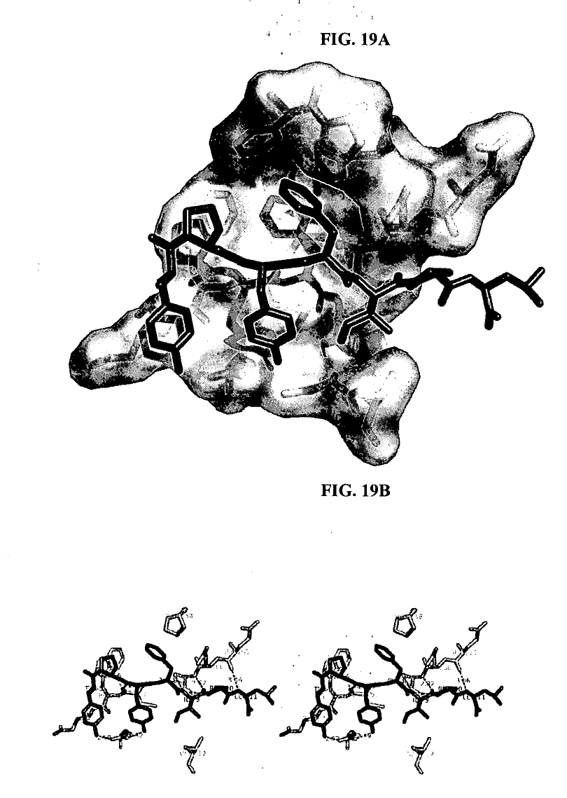

Data were collected at 100K on beam line ID-17, IMCA-CAT, Advanced Photon Source, Argonne National Labs and processed using HKL2000 (Otwinowski et. al., (1997) Methods in Enzymology, Macromolecular Crystallography, Part A; C. W. Carter, Jr. and R. M. Sweet, Eds.; Academic Press: New York, 276:307-326) (Table 6). The structure was determined by molecular replacement using the program EPMR (Kissinger et. al. (1999) Acta Crystallog. Sect. D, 55:484-491). in space group P2₁2₁2₁ with one BACE dimer per asymmetric unit. The starting model used was a BACE dimer (without inhibitor) from a previously determined structure. Strong, unambiguous electron density was observed in the initial maps for active site inhibitor DPH-153979 and for the PYF core of the exosite peptide for both molecules in the asymmetric unit. The exosite peptide bound in a shallow groove on the surface of the protein near residues 316-331 (Table 9). Iterative cycles of model building and map generation using the programs CNX and QUANTA (Accelrys Software, Inc.) allowed for the building of additional exosite peptide residues. The final model includes a BACE dimer, one DPH-153979 per molecule, 161 water molecules and exosite peptide TTYPYFIP in monomer A and YPYFIPL in monomer B (Table 6) (FIGS. 18B and 19). Residues that define the exosite peptide binding site and are within 6 Å of any exosite peptide atom are: E316, K317, F318, P319, F322, G325, E326, Q327, L328, V329, C330, W331, Q332, A333, T335, D372, V373, A374, S376, D378, D379, C380, Y381 (SEQ ID NO:112) where the amino acid numbering is based on GenBank Accession No. NP_{—}036236. Table 8 lists the coordinates of T7-BACE1(A14-T454/R56K/R57K) complexed with DPH-153979 and BMS-561871 and Table 9 shows the correlation of the amino acid sequence numbering between BACE-1 (GenBank Accession No. NP-036236) and Hong et. al. (Hong et. al., (2000) Science, 290:150-153) for residues within 6 Å of exosite peptide BMS-561871.

TABLE 6
Data collection and refinement statistics.

| | BMS-597041 | BMS-561871 |
|---|---|---|
| Resolution range (Å) | 50.0-2.5 | 50.0-2.2 |
| Space Group | P2₁ | P2₁2₁2₁ |
| Cell Dimensions | | |
| a (Å) | 60.2 | 86.5 |
| b (Å) | 130.6 | 93.9 |
| c (Å) | 64.1 | 131.7 |
| α (°) | 90 | 90 |
| β (°) | 91.8 | 90 |
| γ (°) | 90 | 90 |
| Wavelength (Å) | 1.54 | 1.00 |
| Unique Reflections | 31,597 | 55,031 |
| Highest Resolution Shell (Å) | 2.59-2.50 | 2.28-2.20 |
| Redundancy^{a} | 3.6 (3.4) | 7.2 (7.0) |
| I/σI^{a} | 15.4 (2.9) | 21.6 (4.6) |
| Completeness^{a} (%) | 92.7 (96.6) | 100 (99.9) |
| Rmerge^{a} (%) | 12.6 (48.5) | 11.7 (50.4) |
| Rwork (%) | | 28.7 |
| Rfree (%) | | 32.6 |
| RMSD Bond lengths (Å) | | 0.007 |
| RMSD Bond angles (°) | | 1.1 |
| Number of non-hydrogen atoms | | |
| Protein | | 6096 |
| Inhibitors | | 106 |
| Exosite Peptides | | 137 |
| Water | | 181 |
| Mean B factors (Å²) | | |
| Protein | | 27.7 |
| Inhibitors | | 27.2 |
| Exosite Peptides | | 35.7 |

^{a}Values in parentheses correspond to the highest-resolution shell.

TABLE 9
Correlation of the amino acid sequence numbering between
BACE-1 GenBank Accession No. NP_036236 and the
numbering scheme of Hong et. al. for the residues within
6 Å of exosite peptide BMS-561871.
The Hong et. al. numbering scheme is used in Tables 7-8.

| Amino Acid | GenBank Accession No. | |
|---|---|---|
| Residue | NP_036236 | Hong et al. |
| Glu | 316 | 255 |
| Lys | 317 | 256 |
| Phe | 318 | 257 |
| Pro | 319 | 258 |
| Phe | 322 | 261 |
| Gly | 325 | 264 |
| Glu | 326 | 265 |
| Gln | 327 | 266 |
| Leu | 328 | 267 |
| Val | 329 | 268 |
| Cys | 330 | 269 |
| Trp | 331 | 270 |
| Gln | 332 | 271 |
| Ala | 333 | 272 |
| Thr | 335 | 274 |
| Asp | 372 | 311 |
| Val | 373 | 312 |
| Ala | 374 | 313 |
| Ser | 376 | 315 |
| Asp | 378 | 317 |
| Asp | 379 | 318 |
| Cys | 380 | 319 |
| Tyr | 381 | 320 |

Various publications are cited herein that are hereby incorporated by reference in their entirety.

As will be apparent to those skilled in the art to which the invention pertains, the present invention may be embodied in forms other than those specifically disclosed above without departing from the scope and spirit of the invention.

What is claimed is:

1. An isolated peptide that is capable of specifically binding to a BACE exosite peptide binding site, wherein said BACE exosite binding site comprises amino acid residues that are within 6.0 Å of each of exosite peptide atoms E316, K317, F318, P319, F322, G325, E326, Q327, L328, V329, C330, W331, Q332, A333, T335, D372, V373, A374, S376, D378, D379, C380 and Y381 (SEQ ID NO:113).

2. The isolated peptide of claim 1 wherein the isolated peptide comprises an amino acid sequence motif selected from the group consisting of YPYF (SEQ ID NO:1), XYPYF (SEQ ID NO:3), XYPYFX (SEQ ID NO:4), XYPYFXX (SEQ ID NO:5), YPYFX (SEQ ID NO:6) YPYFXX (SEQ ID NO:7), HYPYF (SEQ ID NO:8), YPYFI (SEQ ID NO:2), YPYFIP (SEQ ID NO:9), YPYFIPL (SEQ ID NO:10), YPYFLPI (SEQ ID NO:11), YPYFXPI (SEQ ID NO:12), YPYFXPX (SEQ ID NO:13), HYPYFIP (SEQ ID NO:14) YPYFL (SEQ ID NO:15), YPYFLP (SEQ ID NO:16), HYPYFLP (SEQ ID NO:17), HYPYFIPL (SEQ ID NO:18), LTTYPYFIPLP (SEQ ID NO:44); TTYPYFIPLP (SEQ ID NO:45), TYPYFIPLP (SEQ ID NO:46), NLTTYPYFIPL (SEQ ID NO:48), YPYFIAL (SEQ ID NO:49), YPYFIPA (SEQ ID NO:50) YPYFIPB (SEQ ID NO:52), HYPYFI (SEQ ID NO:54), NLTTYPYFIPLP (SEQ ID NO:19) ALYPYFLPISAK (SEQ ID NO:20), WPXFI (SEQ ID NO:21), ETWPRFIPYHALTQQTLKHQQHT (SEQ ID NO:22), TAEYESRTARTAPPAPTQHWPFFIRST (SEQ ID NO:23), HWPPFFIRS (SEQ ID NO:57), YPBFIPL (SEQ ID NO:51) and YPYFIP (SEQ ID NO:10), wherein X is a naturally or nonnaturally occurring amino acid residue, and wherein B is a benzophenone group.

3. The isolated peptide of claim 2 wherein one or more amino acid residues of the amino acid sequence motif is replaced with a conservative amino acid substitution.

4. The isolated peptide of claim 1 wherein the isolated peptide consists of 5 to 30 amino acids.

5. The isolated peptide of claim 1 wherein the isolated peptide contains one or more modified amino acid residues.

6. The isolated peptide of claim 4 wherein the isolated peptide contains a label selected from the group consisting of a fluorescent label, a chromophore label, a radiolabel and a biotin label.

7. The isolated peptide of claim 1 wherein the isolated peptide comprises a terminal modification that enhances the resistance of the peptide to proteolysis.

8. The isolated peptide of claim 1 wherein the isolated peptide is cyclic.

9. A composition comprising the isolated peptide of claim 1 and a pharmaceutically acceptable carrier.

10. A method of identifying a peptide that specifically binds to a BACE exosite binding site comprising:
(a) contacting BACE with at least one peptide; and
(b) determining whether the peptide specifically binds to the BACE exosite binding site, wherein said BACE exosite binding site comprises amino acid residues that are within 6.0 Å of each of exosite peptide atoms E316, K317, F318, P319, F322, G325, E326, Q327, L328, V329, C330, W331, Q332, A333, T335, D372, V373, A374, S376, D378, D379, C380 and Y381 (SEQ ID NO:113).

11. A method of identifying a modulator of BACE which comprises:
(a) providing the structural coordinates of a BACE exosite binding site provided in one of Tables 7-8 defining a three-dimensional structure of a BACE exosite binding site;
(b) using the three-dimensional structure to design or select a test compound by computer modeling;
(c) synthesizing or acquiring the test compound; and
(d) contacting the test compound with BACE to determine the ability of the test compound to modulate a biological activity of BACE.

12. The method of claim 11 wherein the biological activity is APP processing or beta-amyloid production.

13. A crystalline form comprising a BACE polypeptide wherein said BACE polypeptide comprises a BACE exosite binding site.

14. The crystalline form of claim 13 wherein the crystalline form has lattice parameters of a=60.2 Å, b=130.6 Å, c=64.1 Å, α=γ=90°, β=91.8° and a unit cell variability of 30% in all dimensions.

15. The crystalline form of claim 13 wherein the crystalline form has lattice parameters of a=86.5 Å, b=93.9 Å, c=131.7 Å, α==γ=90° and a unit cell variability of 30% in all dimensions.

16. The crystalline form of claim 13 wherein the crystalline form has symmetry consistent with a monoclinic space group P2₁.

17. The crystalline form of claim 13 wherein the crystalline from comprises a structure defined by all or a portion of the coordinates of Table 7 ±a root mean square deviation from the Cα atoms of less than 0.5 Å.

18. The crystalline form of claim 13 wherein the crystalline from comprises a structure defined by all or a portion of the coordinates of Table 8 ±a root mean square deviation from the Cα atoms of less than 0.5 Å.

19. The crystalline form of claim 13 wherein the crystalline form has symmetry consistent with the space group P2₁2₁2₁.


SEQUENCE LISTING (113 sequences)

SEQ ID NO: 1 - protein, 4 aa, Artificial
  Synthesized Peptide
YPYF

SEQ ID NO: 2 - protein, 5 aa, Artificial
  Synthesized Peptide
YPYFI

SEQ ID NO: 3 - protein, 5 aa, Artificial
  Synthesized Peptide
  misc_feature (1)..(1): Xaa can be any naturally occurring amino acid
XYPYF

SEQ ID NO: 4 - protein, 6 aa, Artificial
  Synthesized Peptide
  misc_feature (1)..(1): Xaa can be any naturally occurring amino acid
  misc_feature (6)..(6): Xaa can be any naturally occurring amino acid
XYPYFX

SEQ ID NO: 5 - protein, 7 aa, Artificial
  Synthesized Peptide
  misc_feature (1)..(1): Xaa can be any naturally occurring amino acid
  misc_feature (6)..(7): Xaa can be any naturally occurring amino acid
XYPYFXX

SEQ ID NO: 6 - protein, 5 aa, Artificial
  Synthesized Peptide
  misc_feature (5)..(5): Xaa can be any naturally occurring amino acid
YPYFX

SEQ ID NO: 7 - protein, 6 aa, Artificial
  Synthesized Peptide
  misc_feature (5)..(6): Xaa can be any naturally occurring amino acid
YPYFXX

SEQ ID NO: 8 - protein, 5 aa, Artificial
  Synthesized Peptide
HYPYF

SEQ ID NO: 9 - protein, 6 aa, Artificial
  Synthesized Peptide
YPYFIP

SEQ ID NO: 10 - protein, 7 aa, Artificial
  Synthesized Peptide
  MOD_RES (1)..(1): wherein amino acid 1 may be acetylated
  MOD_RES (7)..(7): wherein amino acid 7 may end with an amine
YPYFIPL

SEQ ID NO: 11 - protein, 7 aa, Artificial
  Synthesized Peptide
YPYFLPI

SEQ ID NO: 12 - protein, 7 aa, Artificial
  Synthesized Peptide
  misc_feature (5)..(5): Xaa can be any naturally occurring amino acid
YPYFXPI

SEQ ID NO: 13 - protein, 7 aa, Artificial
  Synthesized Peptide
  misc_feature (5)..(5): Xaa can be any naturally occurring amino acid
  misc_feature (7)..(7): Xaa can be any naturally occurring amino acid
YPYFXPX

SEQ ID NO: 14 - protein, 7 aa, Artificial
  Synthesized Peptide
HYPYFIP

SEQ ID NO: 15 - protein, 5 aa, Artificial
  Synthesized Peptide
YPYFL

SEQ ID NO: 16 - protein, 6 aa, Artificial
  Synthesized Peptide
YPYFLP

SEQ ID NO: 17 - protein, 7 aa, Artificial
  Synthesized Peptide
HYPYFLP

SEQ ID NO: 18 - protein, 8 aa, Artificial
  Synthesized Peptide
HYPYFIPL

SEQ ID NO: 19 - protein, 12 aa, Artificial
  Synthesized Peptide
  MOD_RES (12)..(12): wherein amino acid ends with an amine
NLTTYPYFIPLP

SEQ ID NO: 20 - protein, 12 aa, Artificial
  Synthesized Peptide
  MOD_RES (12)..(12): wherein the amino acid ends with an amine
ALYPYFLPISAK

SEQ ID NO: 21 - protein, 5 aa, Artificial
  Synthesized Peptide
  misc_feature: wherein Xaa is any amino acid
  misc_feature (3)..(3): Xaa can be any naturally occurring amino acid
WPXFI

SEQ ID NO: 22 - protein, 23 aa, Artificial
  Synthesized Peptide
ETWPRFIPYHALTQQTLKHQQHT

SEQ ID NO: 23 - protein, 27 aa, Artificial
  Synthesized Peptide
TAEYESRTARTAPPAPTQHWPFFIRST

SEQ ID NO: 24 - protein, 12 aa, Artificial
  Synthesized Peptide
QNHYPYFIAVPI

SEQ ID NO: 25 - protein, 12 aa, Artificial
  Synthesized Peptide
EGNKHYPYFIKV

SEQ ID NO: 26 - protein, 12 aa, Artificial
  Synthesized Peptide
THSHYPYFIELE

SEQ ID NO: 27 - protein, 12 aa, Artificial
  Synthesized Peptide
QQYPYFIPVIRP

SEQ ID NO: 28 - protein, 12 aa, Artificial
  Synthesized Peptide
HYPYFLPLHTPK

SEQ ID NO: 29 - protein, 24 aa, Artificial
  Synthesized Peptide
AMLDGAPTNRNSQHYPYFLPIATV

SEQ ID NO: 30 - protein, 23 aa, Artificial
  Synthesized Peptide
LPVYDTTAPTHYPYFLPLPRISP

SEQ ID NO: 31 - protein, 12 aa, Artificial
  Synthesized Peptide
SQLQHYPYFRPL

SEQ ID NO: 32 - protein, 12 aa, Artificial
  Synthesized Peptide
YIPHYPYFIRLN

SEQ ID NO: 33 - protein, 23 aa, Artificial
  Synthesized Peptide
KMHSMINQLGTRHYPYFREINDY

SEQ ID NO: 34 - protein, 12 aa, Artificial
  Synthesized Peptide
GSTKSYPYFIHT

SEQ ID NO: 35 - protein, 23 aa, Artificial
  Synthesized Peptide
DIWNGAKAPKNSMYPYFIPSSLK

SEQ ID NO: 36 - protein, 27 aa, Artificial
  Synthesized Peptide
ISVINQPAQNMHPRQMTAYPYFRPISR

SEQ ID NO: 37 - protein, 23 aa, Artificial
  Synthesized Peptide
DVYPYFVSSNEGHSIRHKGNNSL

SEQ ID NO: 38 - protein, 33 aa, Artificial
  Synthesized Peptide
YPYFIDSHPPKELMPHSWVQSKYPASPQTHTTY

SEQ ID NO: 39 - protein, 12 aa, Artificial
  Synthesized Peptide
GYPYFLNLKNSH

SEQ ID NO: 40 - protein, 12 aa, Artificial
  Synthesized Peptide
NSYPYFIHLSNP

SEQ ID NO: 41 - protein, 12 aa, Artificial
  Synthesized Peptide
HDYPYFMMLTGH

SEQ ID NO: 42 - protein, 12 aa, Artificial
  Synthesized Peptide
QIETYPYFLPIL

SEQ ID NO: 43 - protein, 12 aa, Artificial
  Synthesized Peptide
YYPYFISTAREV

SEQ ID NO: 44 - protein, 11 aa, Artificial
  Synthesized Peptide
  MOD_RES (1)..(1): ACETYLATION
  MOD_RES (11)..(11): wherein amino acid 11 may end with an amine
LTTYPYFIPLP

SEQ ID NO: 45 - protein, 10 aa, Artificial
  Synthesized Peptide
  MOD_RES (1)..(1): ACETYLATION
  MOD_RES (10)..(10): wherein amino acid 10 may end with an amine
TTYPYFIPLP

SEQ ID NO: 46 - protein, 9 aa, Artificial
  Synthesized Peptide
  MOD_RES (1)..(1): ACETYLATION
  MOD_RES (9)..(9): wherein amino acid 9 may end with an amine
TYPYFIPLP

SEQ ID NO: 47 - protein, 8 aa, Artificial
  Synthesized Peptide
  MOD_RES (1)..(1): ACETYLATION
  MOD_RES (8)..(8): wherein amino acid 8 may end with an amine
YPYFIPLP

SEQ ID NO: 48 - protein, 11 aa, Artificial
  Synthesized Peptide
  MOD_RES (11)..(11): wherein amino acid 11 may end with an amine
NLTTYPYFIPL

SEQ ID NO: 49 - protein, 7 aa, Artificial
  Synthesized Peptide
  MOD_RES (1)..(1): ACETYLATION
  MOD_RES (7)..(7): wherein amino acid 7 may end with an amine
YPYFIAL

SEQ ID NO: 50 - protein, 7 aa, Artificial
  Synthesized Peptide
  MOD_RES (1)..(1): wherein amino acid 1 may be acylated
  MOD_RES (7)..(7): wherein amino acid 7 may end with an amine
YPYFIPA

SEQ ID NO: 51 - protein, 6 aa, Artificial
  Synthesized Peptide
  MOD_RES (1)..(1): wherein amino acid 1 may be acylated
  MOD_RES (2)..(3): wherein Pro2 may be joined to Phe3 by a p-benzoyl phenylalanine
YPFIPL

SEQ ID NO: 52 - protein, 6 aa, Artificial
  Synthesized Peptide
  MOD_RES (1)..(1): ACETYLATION
  MOD_RES (6)..(6): wherein amino acid 6 may end with a p-benzoyl phenalanine
YPYFIP

SEQ ID NO: 53 - protein, 7 aa, Artificial
  Synthesized Peptide
LYPPYIF

SEQ ID NO: 54 - protein, 6 aa, Artificial
  motif
HYPYFI

SEQ ID NO: 55 - protein, 16 aa, Artificial
  Synthesized Peptide
  misc_feature (1)..(5): Xaa can be any naturally occurring amino acid
  misc_feature (12)..(16): Xaa can be any naturally occurring amino acid
XXXXXHYPYFIXXXXX

SEQ ID NO: 56 - protein, 18 aa, Artificial
  Synthesized Peptide
  misc_feature (2)..(6): Xaa can be any naturally occurring amino acid
  misc_feature (13)..(17): Xaa can be any naturally occurring amino acid
CXXXXXHYPYFIXXXXXC

SEQ ID NO: 57 - protein, 8 aa, Artificial
  Synthesized Peptide
  MOD_RES (1)..(1): wherein amino acid 1 may be acetylated
  MOD_RES (8)..(8): wherein amino acid 8 may end with an amine
HWPFFIRS

SEQ ID NO: 58 - protein, 16 aa, Artificial
  Synthesized Peptide
TDQPKHYPYFIPSPHS

SEQ ID NO: 59 - protein, 16 aa, Artificial
  Synthesized Peptide
THQPKHYPYFIPYHHD

SEQ ID NO: 60 - protein, 16 aa, Artificial
  Synthesized Peptide
MDHEKHYPYFIEYKHV

SEQ ID NO: 61 - protein, 18 aa, Artificial
  Synthesized Peptide
CTEANKHYPYFIPRHSSC

SEQ ID NO: 62 - protein, 16 aa, Artificial
  Synthesized Peptide
HSLAPHYPYFIDLHST

SEQ ID NO: 63 - protein, 16 aa, Artificial
  Synthesized Peptide
GSQALHYPYFIPYHKH

SEQ ID NO: 64 - protein, 18 aa, Artificial
  Synthesized Peptide
CTNKHDHYPYFIRPGEFC

SEQ ID NO: 65 - protein, 18 aa, Artificial
  Synthesized Peptide
CENKHDHYPYFISAGNYC

SEQ ID NO: 66 - protein, 18 aa, Artificial
  Synthesized Peptide
CQTKVMHYPYFIREGVTC

SEQ ID NO: 67 - protein, 18 aa, Artificial
  Synthesized Peptide
CGPKHLHYPYFISATSRC

SEQ ID NO: 68 - protein, 18 aa, Artificial
  Synthesized Peptide
CAAKHSHYPYFIPACSSC

SEQ ID NO: 69 - protein, 18 aa, Artificial
  Synthesized Peptide
CASTYPHYPYFIATCKTC

SEQ ID NO: 70 - protein, 18 aa, Artificial
  Synthesized Peptide
CAEAKQHYPYFIKWCKTC

SEQ ID NO: 71 - protein, 18 aa, Artificial
  Synthesized Peptide
CAEAKGHYPYFICTTGNC

SEQ ID NO: 72 - protein, 17 aa, Artificial
  Synthesized Peptide
CAQAREHYPYFIDLRTV

SEQ ID NO: 73 - protein, 18 aa, Artificial
  Synthesized Peptide
CAKAPRHYPYFISAQNAW

SEQ ID NO: 74 - protein, 18 aa, Artificial
  Synthesized Peptide
CAKASHHYPYFINLANNG

SEQ ID NO: 75 - protein, 18 aa, Artificial
  Synthesized Peptide
CARAITHYPYFIPYCEEC

SEQ ID NO: 76 - protein, 16 aa, Artificial
  Synthesized Peptide
AVSQTHYPYFIPLSQA

SEQ ID NO: 77 - protein, 18 aa, Artificial
  Synthesized Peptide
CEDRPTHYPYFISLNKQC

SEQ ID NO: 78 - protein, 18 aa, Artificial
  Synthesized Peptide
CKTQDNHYPYFISLKKAC

SEQ ID NO: 79 - protein, 18 aa, Artificial
  Synthesized Peptide
CQTKHQHYPYFISLTDAC

SEQ ID NO: 80 - protein, 18 aa, Artificial
  Synthesized Peptide
CTKAHTHYPYFISNSKIC

SEQ ID NO: 81 - protein, 18 aa, Artificial
  Synthesized Peptide
CHHKHTHYPYFIPNTKSC

SEQ ID NO: 82 - protein, 18 aa, Artificial
  Synthesized Peptide
CSQHHTHYPYFIPSNGMC

SEQ ID NO: 83 - protein, 18 aa, Artificial
  Synthesized Peptide
CAVEARHYPYFINTCSNC

SEQ ID NO: 84 - protein, 18 aa, Artificial
  Synthesized Peptide
CSVVNRHYPYFINNSSKC

SEQ ID NO: 85 - protein, 18 aa, Artificial
  Synthesized Peptide
CTGCARHYPYFIEVSTQW

SEQ ID NO: 86 - protein, 18 aa, Artificial
  Synthesized Peptide
CSNASHHYPYFISTHSTC

SEQ ID NO: 87 - protein, 18 aa, Artificial
  Synthesized Peptide
CSNPTGHYPYFISPQGTC

SEQ ID NO: 88 - protein, 18 aa, Artificial
  Synthesized Peptide
CNSTPRHYPYFISVNSTC

SEQ ID NO: 89 - protein, 18 aa, Artificial
  Synthesized Peptide
CGVQLVHYPYFLPANSTC

SEQ ID NO: 90 - protein, 18 aa, Artificial
  Synthesized Peptide
CARTPSHYPYFISLPDRG

SEQ ID NO: 91 - protein, 18 aa, Artificial
  Synthesized Peptide
CSAGHNHYPYFITLPGYG

SEQ ID NO: 92 - protein, 18 aa, Artificial
  Synthesized Peptide
CASQDYHYPYFIPSPAWG

SEQ ID NO: 93 - protein, 16 aa, Artificial
  Synthesized Peptide
ELPFQHYPYFIDLPPV

SEQ ID NO: 94 - protein, 16 aa, Artificial
  Synthesized Peptide
MHPNPHYPYFIPLPTR

SEQ ID NO: 95 - protein, 18 aa, Artificial
  Synthesized Peptide
CDSCVTHYPYFINTPYKY

SEQ ID NO: 96 - protein, 18 aa, Artificial
  Synthesized Peptide
CAKPKQHYPYFICYPHEC

SEQ ID NO: 97 - protein, 16 aa, Artificial
  Synthesized Peptide
INKTQHYPYFIEYPFH

SEQ ID NO: 98 - protein, 18 aa, Artificial
  Synthesized Peptide
CPNTQHHYPYFIKVGEHC

SEQ ID NO: 99 - protein, 18 aa, Artificial
  Synthesized Peptide
CPDIAHHYPYFIDSKSHC

SEQ ID NO: 100 - protein, 18 aa, Artificial
  Synthesized Peptide
CQPTRHHYPYFIDVTGRC

SEQ ID NO: 101 - protein, 18 aa, Artificial
  Synthesized Peptide
CQNNHHHYPYFITPTHVC

SEQ ID NO: 102 - protein, 18 aa, Artificial
  Synthesized Peptide
CTTTHEHYPYFIDPREAC

SEQ ID NO: 103 - protein, 18 aa, Artificial
  Synthesized Peptide
CTTPSRHYPYFIDQLGHC

SEQ ID NO: 104 - protein, 18 aa, Artificial
  Synthesized Peptide
CNANHTHYPYFIDISRKC

SEQ ID NO: 105 - protein, 16 aa, Artificial
  Synthesized Peptide
QFTHKHYPYFINISPG

SEQ ID NO: 106 - protein, 18 aa, Artificial
  Synthesized Peptide
CNMPHSHYPYFINPHQSC

SEQ ID NO: 107 - protein, 9 aa, Artificial
  Synthesized Peptide
  MOD_RES (1)..(1): wherein amino acid may be modified with a 7-methoxycoumarin-4-acetyl group
  MOD_RES (9)..(9): Wherein amino acid 9 may be modified with a carboxylated dinitrophenyl group
EVNLDAEFK

SEQ ID NO: 108 - protein, 6 aa, Artificial
  Synthesized peptide
  MOD_RES (6)..(6): wherein amino acid 6 ends with a p-benzoyl phenylalanine-2,3 dipropionic acid to which an Alexa488 is bound
YPYFIP

SEQ ID NO: 109 - protein, 6 aa, Artificial
  Synthesized peptide
  MOD_RES (2)..(3): wherein Pro 2 and Phe 3 are joined by a p-benzoyl phenylalanine
  MOD_RES (6)..(6): wherein amino acid 6 ends with a 2,3 dipropionic acid to which an Alexa488 is bound
YPFIPL

SEQ ID NO: 110 - DNA, 1390 bp, Artificial
  Synthesized Peptide
catatggctagcatgactggtggacagcaaatgggtcgcggatccgcgggagtgctgcctgcccacggcacccagcacggcatccggctgcccctgcgcagcggcctggggggcgcccccctggggctgcggctgccccgggagaccgacgaagagcccgaggagcccggccggaggggcagctttgtggagatggtggacaacctgaggggcaagtcggggcagggctactacgtggagatgaccgtgggcagccccccgcagacgctcaacatcctggtggatacaggcagcagtaactttgcagtgggtgctgccccccaccccttcctgcatcgctactaccagaggcagctgtccagcacataccgggacctccggaagggtgtgtatgtgccctacacccagggcaagtgggaaggggagctgggcaccgacctggtaagcatcccccatggccccaacgtcactgtgcgtgccaacattgctgccatcactgaatcagacaagttcttcatcaacggctccaactgggaaggcatcctggggctggcctatgccgagattgccaggcctgacgactccctggagcctttctttgactctctggtaaagcagacccacgttcccaacctcttctccctgcagctttgtggtgctggcttccccctcaaccagtctgaagtgctggcctctgtcggagggagcatgatcattggaggtatcgaccactcgctgtacacaggcagtctctggtatacacccatccggcgggagtggtattatgaggtcatcattgtgcgggtggagatcaatggacaggatctgaaaatggattgcaaggagtacaactatgacaagagcattgtggacagtggcaccaccaaccttcgtttgcccaagaaagtgtttgaagctgcagtcaaatccatcaaggcggcctcctccacggagaagttccctgatggtttctggctaggagagcagctggtgtgctggcaagcaggcaccaccccttggaacattttcccagtcatctcactctacctaatgggtgaggttaccaaccagtccttccgcatcaccatccttccgcagcaatacctgcggccagtggaagatgtggccacgtcccaagacgactgttacaagtttgccatctcacagtcatccacgggcactgttatgggagctgttatcatggagggcttctacgttgtctttgatcgggcccgaaaacgaattggctttgctgtcagcgcttgccatgtgcacgatgagttcaggacggcagcggtggaaggcccttttgtcaccttggacatggaagactgtggctacaacattccacagacagatgagtcaacctgaggatccgaattcgagctcc

SEQ ID NO: 111 - protein, 455 aa, Artificial
  Synthesized Peptide
MASMTGGQQMGRGSAGVLPAHGTQHGIRLPLRSGLGGAPLGLRLPRETDEEPEEPGRRGSFVEMVDNLRGKSGQGYYVEMTVGSPPQTLNILVDTGSSNFAVGAAPHPFLHRYYQRQLSSTYRDLRKGVYVPYTQGKWEGELGTDLVSIPHGPNVTVRANIAAITESDKFFINGSNWEGILGLAYAEIARPDDSLEPFFDSLVKQTHVPNLFSLQLCGAGFPLNQSEVLASVGGSMIIGGIDHSLYTGSLWYTPIRREWYYEVIIVRVEINGQDLKMDCKEYNYDKSIVDSGTTNLRLPKKVFEAAVKSIKAASSTEKFPDGFWLGEQLVCWQAGTTPWNIFPVISLYLMGEVTNQSFRITILPQQYLRPVEDVATSQDDCYKFAISQSSTGTVMGAVIMEGFYVVFDRARKRIGFAVSACHVHDEFRTAAVEGPFVTLDMEDCGYNIPQTDEST

SEQ ID NO: 112 - DNA, 1390 bp, Artificial
  Synthesized Peptide
catatggctagcatgactggtggacagcaaatgggtcgcggatccgcgggagtgctgcctgcccacggcacccagcacggcatccggctgcccctgcgcagcggcctggggggcgcccccctggggctgcggctgccccgggagaccgacgaagagcccgaggagcccggcaagaagggcagctttgtggagatggtggacaacctgaggggcaagtcggggcagggctactacgtggagatgaccgtgggcagccccccgcagacgctcaacatcctggtggatacaggcagcagtaactttgcagtgggtgctgccccccaccccttcctgcatcgctactaccagaggcagctgtccagcacataccgggacctccggaagggtgtgtatgtgccctacacccagggcaagtgggaaggggagctgggcaccgacctggtaagcatcccccatggccccaacgtcactgtgcgtgccaacattgctgccatcactgaatcagacaagttcttcatcaacggctccaactgggaaggcatcctggggctggcctatgccgagattgccaggcctgacgactccctggagcctttctttgactctctggtaaagcagacccacgttcccaacctcttctccctgcagctttgtggtgctggcttccccctcaaccagtctgaagtgctggcctctgtcggagggagcatgatcattggaggtatcgaccactcgctgtacacaggcagtctctggtatacacccatccggcgggagtggtattatgaggtcatcattgtgcgggtggagatcaatggacaggatctgaaaatggattgcaaggagtacaactatgacaagagcattgtggacagtggcaccaccaaccttcgtttgcccaagaaagtgtttgaagctgcagtcaaatccatcaaggcggcctcctccacggagaagttccctgatggtttctggctaggagagcagctggtgtgctggcaagcaggcaccaccccttggaacattttcccagtcatctcactctacctaatgggtgaggttaccaaccagtccttccgcatcaccatccttccgcagcaatacctgcggccagtggaagatgtggccacgtcccaagacgactgttacaagtttgccatctcacagtcatccacgggcactgttatgggagctgttatcatggagggcttctacgttgtctttgatcgggcccgaaaacgaattggctttgctgtcagcgcttgccatgtgcacgatgagttcaggacggcagcggtggaaggcccttttgtcaccttggacatggaagactgtggctacaacattccacagacagatgagtcaacctgaggatccgaattcgagctcc

SEQ ID NO: 113 - protein, 455 aa, Artificial
  Synthesized Peptide
MASMTGGQQMGRGSAGVLPAHGTQHGIRLPLRSGLGGAPLGLRLPRETDEEPEEPGKKGSFVEMVDNLRGKSGQGYYVEMTVGSPPQTLNILVDTGSSNFAVGAAPHPFLHRYYQRQLSSTYRDLRKGVYVPYTQGKWEGELGTDLVSIPHGPNVTVRANIAAITESDKFFINGSNWEGILGLAYAEIARPDDSLEPFFDSLVKQTHVPNLFSLQLCGAGFPLNQSEVLASVGGSMIIGGIDHSLYTGSLWYTPIRREWYYEVIIVRVEINGQDLKMDCKEYNYDKSIVDSGTTNLRLPKKVFEAAVKSIKAASSTEKFPDGFWLGEQLVCWQAGTTPWNIFPVISLYLMGEVTNQSFRITILPQQYLRPVEDVATSQDDCYKFAISQSSTGTVMGAVIMEGFYVVFDRARKRIGFAVSACHVHDEFRTAAVEGPFVTLDMEDCGYNIPQTDEST